(19) United States
(12) Patent Application Publication
(10) Pub. No.: US 2022/0151827 A1
(43) Pub. Date: May 19, 2022

(54) Ophthalmic Compositions, and Ocular Uses Thereof, of Indigo Carmine

(71) Applicant: Minas Theodore Coroneo, Vaucluse, New South Wales (AU)

(72) Inventor: Minas Theodore Coroneo, Vaucluse, New South Wales (AU)

(21) Appl. No.: 17/439,282

(22) PCT Filed: Mar. 13, 2020
(86) PCT No.: PCT/AU2020/050238

§ 371 Date: Sep. 14, 2021

Related U.S. Application Data
(60) Provisional application No. 62/842,164, filed on May 2, 2019.
(60) Provisional application No. 62/819,336, filed on Mar. 15, 2019.

(51) Int. Cl. A61F 9/00; A61K 49/00; A61F 9/007

(52) U.S. Cl. CPC A61F 9/0008; A61F 9/00781; A61K 49/0021

(57) ABSTRACT

An ophthalmic composition comprising Indigo Carmine, or Indigo Carmine and Trypan Blue, for identification of intraocular structures and membranes within the eye, and methods of delivering and using the same, for surgical treatments of the eye, including glaucoma and cataract surgery.

### CROSS REFERENCE

This application claims the benefit of priority from U.S. Provisional Application No. 62/819,336, filed on Mar. 15, 2019, and further claims the benefit of priority from U.S. Provisional Application No. 62/842,164, filed on May 2, 2019. Each of the foregoing related applications, in its entirety, is incorporated herein by reference.

### TECHNICAL FIELD

This relates to ophthalmic compositions comprising Indigo Carmine or Indigo Carmine and Trypan Blue, and methods of delivering and using the same, particularly methods of using the same during ocular surgical procedures, such as during surgical procedures to treat patients suffering from glaucoma and/or cataract.

### BACKGROUND

Glaucoma is an eye disease in which inappropriate pressure (usually elevated) damages retinal ganglion cells, resulting in permanent loss of field of vision. Left untreated, glaucoma can result in blindness, since the peripheral field of vision is lost initially and care is not typically sought until late in the course of the disease, when the more central field of vision is affected. Underlying this loss of visual field is the largely irreversible loss of retinal ganglion cells which apoptose in response to pressure (see, e.g., Tan, J. C., et al., “Mechanosensitivity and the eye: cells coping with the pressure”, Br. J. Ophthalmol., 2006; 90:383-388).

The eye, arguably the most sophisticated “camera” that has ever evolved, is a pressurized organ, and the possible reasons for this include that it has an optical system that must remain precisely aligned despite very rapid eye movements. This optical system includes the cornea anteriorly and the crystalline lens suspended by zonular fibers from the ciliary body complex and in close relation to a diaphragm, the iris in which the pupil, of varying diameter, is located. This dual, anterior lens system is designed to focus light onto the “film plane” of the eye, the photosensitive retina. These optical components must maintain shape and alignment and position in order for a clear image to be formed. Since the eye is subjected to many movements (a “roving eye” effect, coordinated, since both eyes must move in unison for stereoscopic vision) and also to make up for the fact that since the human eye is compact and without compound optics (as in the fly), our field of vision, essential for survival, can be greatly increased by rapidly surveying a scene with eye movements. These eye movements include saccades which represent the fastest movement in the body with angular speeds of about 900°/s (see, e.g., Kandel, E. R., et al., Principles of Neural Science, McGraw Hill, 2000, 510, 784-786). If the eye were not pressurized, it would be impossible to maintain the position of these optical components without some “wobble” effect. Furthermore, some cell membrane transport mechanisms are dependent on pressure gradients, which need to be maintained to sustain normal function (see, e.g., Brenner, B. M., et al., “Transport of Molecules across Renal Glomerular Capillaries”, Physiol. Rev., 1976, 6: 502-534).

Fluid, also referred to as the aqueous humor, is continuously produced inside the eye by the epithelium of the ciliary body, thereby generating pressure as well as providing nutrients and removing waste products from the anterior eye. This fluid leaves the eye by a number of pathways. One fluid exit pathway, the so-called conventional drainage pathway, involves drainage of fluid from the inner eye (where ostensibly the bulk of aqueous humor resides) exiting the eye via the angle between the cornea and the iris (FIG. 1A). In this angle exists the trabecular meshwork (see, e.g., U.S. Pat. No. 6,372,449; Carreon, T., et al., “Aqueous outflow—A continuum from trabecular meshwork to episcleral veins”, Prog. Retin. Eye Res., 2017, 57:108-133; Johnson, M., et al., “Unconventional aqueous humor outflow: A review”, Exp. Eye Res., 2017, 158:94-111; and Carreon, T. A., et al., “Segmental outflow of aqueous humor in mouse and human”, Exp. Eye Res., 2017, 158:59-66), through which fluid filters into the canal of Schlemm (a circumferential channel), thence into the deep scleral plexus and collector channels, and exiting outside the eye into episcleral/aqueous veins on the surface of the sclera (FIGS. 1A and 1B). Since this structural system involved in the conventional drainage pathway (including the trabecular meshwork, canal of Schlemm, deep scleral plexus, collector channels, and episcleral/aqueous veins) is thought to be the main controlling mechanism of fluid egress, responsible for maintaining eye pressure within a relatively narrow range for the life of the individual, it represents the most sophisticated valve in the body. Yet the function of this structural system is poorly understood and attempts to replace or subvert it are at the core of all glaucoma surgery, a branch of eye surgery that has had limited long-term success. A second fluid exit pathway, the uveoscleral or unconventional pathway, also includes passage via cornea, iris and retina (see, e.g., Carreon, T., et al., “Aqueous outflow—A continuum from trabecular meshwork to episcleral veins”, Prog. Retin. Eye Res., 2017, 57:108-133). Fluid exits the posterior aspect of the uveal meshwork, passing through the ciliary muscle, and entering the suprachoroidal space. This pathway is variously estimated to account for 14-54% of outflow in human eyes and is also reduced in glaucoma. Another fluid exit pathway involves pumping fluid out of the eye by the retinal pigment epithelium (see, e.g., Pederson, J. E. et al., “Experimental retinal detachment: V. Fluid movement through the retinal hole”, Arch. Ophthalmol., 1984, 102:136-139), but the relative importance of this pathway is thought to be small.

The cause of elevated pressure in glaucoma (and with increasing age) is not fully understood but is thought to involve a blockage (actual physical obstruction, pathophysiological and molecular changes or a combination) in the pathways that allow fluid outflow from the eye. Specific causes of this impediment remain elusive despite rigorous investigation (see, e.g., Coroneo, M. T., et al., “Electrical and morphological evidence for heterogeneous populations of cultured bovine trabecular meshwork cells”, Exp. Eye Res., 1991, 52:375-88). An early notion was of “silting” of the valve with an increase in the amount and change in nature of extracellular material in the spaces of the endothelial meshwork (or juxtacanalicular tissue (JCT))—where the drainage route is most tortuous, then considered the probable site at which abnormally elevated resistance develops in early primary open angle glaucoma (see, e.g., Watson, P. G., et al., “The place of trabeculectomy in the treatment of glaucoma”, Ophthalmology, 1981, 88:175-96). Increased cellularity and hyalinization in this region have also been noted. Subsequent studies in glaucomatous eyes found decreased Schlemm's canal cross-sectional area, perimeter and length and histopathologic changes in the outer wall of Schlemm's canal including increased collapse and narrowing of collector channels and intrascleral veins along with adhesion of Schlemm's canal endothelium to collector channels orifice walls and herniation of juxtacanalicular tissue with blockage of collector channel orifices (see, e.g., Hann, C. R., et al., “Anatomic changes in Schlemm's canal and collector channels in normal and primary open-angle glaucoma eyes using low and high perfusion pressures”, Invest Ophthalmol. Vis. Sci., 2014 Aug. 19, 55(9):5834-41). This is consistent with the finding that the distal portion of the conventional outflow pathway is responsible for nearly 50% of outflow resistance in low-pressure perfused eyes and about 30% under higher pressures (Id.). A continuum model (FIG. 1B) of ocular outflow resistance in which integrated pathology encompassing the trabecular meshwork, Schlemm's canal, collector channels and distal outflow regions has been proposed (see, e.g., Carreon, T., et al., “Aqueous outflow—A continuum from trabecular meshwork to episcleral veins”, Prog. Retin. Eye Res., 2017, 57:108-133). In this continuum model, reduced or altered trabecular meshwork mechanotransduction occurs due to alteration of soluble mechanosensing molecules or to their deposition. Mechanosensing occurs in the solution phase in the extracellular matrix and mechanotransduction on the cell surface by various channels. Basement membrane degradation is impaired in the trabecular meshwork and Schlemm's canal resulting in the lack of generation of pro- and anti-angiogenic molecules, including certain types of collagen fragments. Downstream, reduced collector channel frequency and/or dimension are observed. The fine regulation of degraded basement membrane protein fragments may be involved in regulation of collector channels and beyond.

Another important factor that may play a role in ocular surgical planning is that aqueous outflow is not uniform but is segmental around the circumference of the drainage angle. Preferential outflow occurs in the nasal and inferior quadrants of the eye (see, e.g., Cha, E. D., et al., “Variations in active outflow along the trabecular outflow pathway”, Exp. Eye Res., 2016, 146:354-60), areas associated with more expanded trabecular meshwork and higher number of collector channels. Accordingly, circumferential flow around Schlemm's canal may be limited as aqueous flow through the trabecular meshwork and Schlemm's canal may be diverted into areas where the collector channels are most abundant to create this segmental flow pattern (FIG. 1B). These segmental variations in outflow facility may be of critical importance in the placement of stents that are inserted in the canal of Sclemm, since placement in the areas of maximum collector channel density will result in improved outflow.

To date, the only proven effective treatment for glaucoma is the lowering of intraocular pressure, which can be achieved pharmacologically, with laser treatment, or with surgery. Until recently, glaucoma surgery has provided variable results, and despite the fact that pressure can be lowered, it is not without risk as sight both visual and/or field of vision) may be reduced by the consequences of the surgery. Glaucoma surgery can be associated with astigmatism, corneal damage, cataract and retinal complications. So while the long-term aim is to protect the optic nerve by lowering pressure, in the short term, vision can be worse as a result of the surgery. Accordingly, these unwanted complications and consequences have required a solution that involves more effective surgery.

In recent years, minimally invasive surgery techniques has revolutionized glaucoma management (see, e.g., U.S. Pat. No. 7,291,125; and Coroneo, M. T., “Suprachoroidal Drainage—Centenarian Progress: An Inventor's Perspective”, Francis, B. A., Sarkisian, S., and Tan, J., Editors, Minimally Invasive Glaucoma Surgery: the Science and the Practice. Thieme, New York, 2016). Minimally invasive surgery for glaucoma, also known as minimally invasive glaucoma surgery (MIGS), has borrowed from the techniques used in modern cataract surgery, in particular the use of small incisions and injectable implants or devices. The design and positioning of these implants or devices has depended on the prevailing view of the major sites of obstruction to aqueous outflow and given the lack of consensus, it is not surprising that different stent designs and techniques have been developed. Broadly, these devices are designed to:
1. bypass trabecular meshwork (stents are typically placed in the canal of Schlemm);
2. bypass the entire conventional drainage system by either:
   a. drainage into the suprachoroidal space (FIG. 1A); or
   b. drainage through the anterior chamber angle, through the wall of the eye and into the subconjunctival space; and/or
3. treat the canal of Schlemm and downstream structures by cannulation and injection of devices, such as ophthalmic viscosurgical devices or drugs.

During the implantation or insertion of these devices, it is helpful to be able to visualize structures in the angle, particularly the trabecular meshwork and the canal of Schlemm. Also, particularly for procedures in which implants are placed in the canal of Schlemm, it would be useful to know the sites of the best downstream drainage in the collector channels, and subsequently, the aqueous veins. This would allow optimal stent placement to take advantage of the downstream pathways of least resistance. This is particularly so because of the known variation in the numbers of collector channels according to location in relation to the 360 degrees of the conventional drainage angle.

Identification of membranes within the eye, whether pathogenic in origin, or those normally found within the eye, is difficult due to the transparent nature of such membranes. As such, these membranes cannot be readily visualized, and the diagnosis and treatment of various conditions associated with ocular membranes is hampered. Structures within the eye, such as the trabecular meshwork and the canal of Schlemm, both of which may be implicated in glaucoma, are difficult to visualize, again due to their relatively transparent nature or lack of pigmentation. Accordingly, a physician or surgeon diagnosing or treating conditions associated with membranes in the eye, with eye structures, or believed to be associated with structures of the eye is hampered by the inability to properly visualize such structures.

Moreover, existing methods of judging location and patency of outflow pathways are not well developed and are either inconsistent or impractical intra-operatively. Immediately after stent placement, lowering intraocular pressure via a paracentesis (small incision through the cornea and into the anterior chamber of the eye) can result in retrograde blood filling of Schlemm's canal (see, e.g., Wirbelauer, C., et al., “Role of Intraoperative Indirect Channelography in Glaucoma Stent Implantation”, Klin. Monbl. Augenheilkd., 2017, 234:1378-1386), however judging this through 360 degrees of the angle during a surgical procedure is currently difficult with available imaging systems. And while the technique of using aqueous humor angiography, in conjunction with the dye indocyanine green, was able to confirm the segmental nature of drainage (see, e.g., Huang, A. S., et al., “Aqueous Angiography: Aqueous Humor Outflow Imaging in Live Human Subjects”, Ophthalmology, 2017, 124:1249-1251), this and other techniques require special imaging equipment (see, e.g., Saraswathy, S., et al., “Aqueous Angiography: Real-Time and Physiologic Aqueous Humor Outflow Imaging”, PLoS One, 2016 Jan. 25, 11(1):e0147176), thereby limiting their accessibility for routine surgery. The disadvantageous necessity of requiring the use of special imaging equipment for such techniques is presumably because of the limited visibility of the dye as it passes through the drainage system. Additionally, in contrast to acidic dyes, which are generally less toxic in tissue interactions (see, e.g., Grant, W. M. et al., Toxicology of the Eye, 4th ed., Springfield, Ill.: Charles C. Thomas, 1993), indocyanine green has the further disadvantage of being a basic dye, thus its use raises concerns of causing further tissue damage.

Another ophthalmic dye, Trypan Blue, while it has been demonstrated to be effective in identifying and visualizing ocular structures, particularly the anterior lens capsule and the trabecular meshwork (see, e.g., U.S. Pat. No. 6,372,449), it is not effective in identifying or visualizing the canal of Schlemm.

Developments in glaucoma and cataract surgery, as noted above, as well as corneal surgery, have necessitated the development of improved imaging techniques in order to obtain improved outcomes. In particular, there is a need for novel ophthalmic dye compositions, and techniques and procedures of using the same, to improve the effectiveness of minimally invasive glaucoma surgery (MIGS), cataract surgery, corneal surgery, including endothelial keratoplasty and small incision lenticule extraction (SMILE), a corneal refractive procedure (see, e.g., Shah, R., et al., “Results of small incision lenticule extraction: all-in-one femtosecond laser refractive surgery”, J. Cataract Refract. Surg., 2011, 37:127-137), combinations of these procedures, and ocular surface diagnostic techniques. Additionally, since cataract and glaucoma can frequently coexist, surgical procedures that address glaucoma can often be carried out in conjunction with (usually following in serial fashion) cataract surgery (see, e.g., Rabin, R. L., et al., “Co-management of cataract and glaucoma in the era of minimally invasive glaucoma surgery”, Curr. Opin. Ophthalmol., 2018, 29:88-95). For this reason, an ophthalmic dye composition, and technique and procedure of using the same, that facilitates both cataract and glaucoma surgery, or specific glaucoma surgical operations, would be very useful.

Accordingly, there is a need for an ophthalmic composition, and methods of delivering and using the same, for effectively visualizing and identifying structures within the eye, particularly ocular structures involved in fluid exit pathways, such as the trabecular meshwork and the canal of Schlemm, and to facilitate diagnosis and surgery, such as glaucoma surgery, minimally invasive glaucoma surgery (MIGS), cataract surgery, cataract and glaucoma surgery, corneal surgery, including endothelial keratoplasty and small incision lenticule extraction (SMILE), corneal refractive procedures, and to facilitate the placement of implants or devices (such as stents) to effect fluid flow.

### Definitions

Terms are used herein as generally used in the art, unless otherwise defined in the following:

The term “ophthalmic device” is understood to refer to an object that is placed on or resides in the eye. The device may provide facilitated fluid (aqueous humor) flow. An ophthalmic device includes, but is not limited to, a stent, or an intraocular lens during cataract surgery.

The terms “identified”, “identifying”, and the like, used in context of an ocular structure(s), are understood to refer herein as the identification of ocular structure(s) via instillation of an ophthalmic dye, such as Indigo Carmine, Trypan Blue, or another ophthalmic dye, whereby the ophthalmic dye facilitates accurately locating the position and identifying ocular structures within the instilled eye. For example, instillation of the ophthalmic dye into an eye facilitates locating and identifying ocular structures in the instilled eye via staining of the ocular structure or by a collection or concentration of the ophthalmic dye in, about, and/or on the ocular structure, thereby facilitating visual identification of the ocular structures. Visual identification of the ocular structures in the instilled eye may be accomplished with or without the assistance of a magnifying powered inspection of the instilled eye (e.g., with a microscope or magnified lens).

### SUMMARY

Some embodiments described herein may provide ophthalmic compositions, and methods of using the same, to identify, mark, or stain an intraocular structure(s) or membrane(s), and/or to treat an ocular disease or condition, such as glaucoma or a cataract.

In one aspect, provided herein is an ophthalmic composition, comprising Indigo Carmine.

In another aspect, provided herein is an ophthalmic composition, comprising Indigo Carmine and Trypan Blue.

In another aspect, provided herein is a method of ocular surgery in a patient in need thereof, comprising instilling an ophthalmic composition comprising Indigo Carmine into the patient's eye.

In another aspect, provided herein is a method of ocular surgery in a patient in need thereof, comprising instilling an ophthalmic composition comprising Indigo Carmine and Trypan Blue into the patient's eye.

In another aspect, provided herein is a method of ocular surgery in a patient in need thereof, comprising: instilling an ophthalmic composition comprising Indigo Carmine and an ophthalmic composition comprising Trypan Blue into the patient's eye.

In another aspect, provided herein is a method of identifying an intraocular structure(s) or membrane(s) within an eye of a patient in need thereof, comprising instilling an ophthalmic composition comprising Indigo Carmine into the patient's eye.

In another aspect, provided herein is a method of identifying an intraocular structure(s) or membrane(s) within an eye of a patient in need thereof, comprising instilling an ophthalmic composition comprising Indigo Carmine and Trypan Blue into the patient's eye.

In another aspect, provided herein is a method of identifying an intraocular structure(s) or membrane(s) within an eye of a patient in need thereof, comprising: instilling an ophthalmic composition comprising Indigo Carmine and an ophthalmic composition comprising Trypan Blue into the patient's eye.

In another aspect, provided herein is a method of introducing an ophthalmic device into an eye of a patient in need thereof, comprising:
i) instilling an ophthalmic composition comprising Indigo Carmine into the patient's eye; and
ii) introducing the ophthalmic device into the instilled eye.

In another aspect, provided herein is a method of introducing an ophthalmic device into an eye of a patient in need thereof, comprising:
i) instilling an ophthalmic composition comprising Indigo Carmine and Trypan Blue into the patient's eye; and
ii) introducing the ophthalmic device into the instilled eye.

In another aspect, provided herein is a method of introducing an ophthalmic device into an eye of a patient in need thereof, comprising:
i) instilling an ophthalmic composition comprising Indigo Carmine and an ophthalmic composition comprising Trypan Blue into the patient's eye; and
ii) introducing the ophthalmic device into the instilled eye.

In another aspect, provided herein is a method of identification of canal of Schlemm within an eye of a patient in need thereof, comprising instilling an ophthalmic composition comprising Indigo Carmine the patient's eye.

In another aspect, provided herein is a method of identification of canal of Schlemm within an eye of a patient in need thereof, comprising instilling an ophthalmic composition comprising Indigo Carmine and Trypan Blue the patient's eye.

In another aspect, provided herein is a method of identification of canal of Schlemm within an eye of a patient in need thereof, comprising instilling an ophthalmic composition comprising Indigo Carmine and an ophthalmic composition comprising Trypan Blue into the patient's eye.

In another aspect, provided herein is a method of cataract extraction and treatment of glaucoma in an eye of a patient in need thereof, comprising:
i) instilling an ophthalmic composition comprising Trypan Blue into the patient's eye;
ii) surgically extracting the cataract of the Trypan Blue instilled eye;
iii) instilling an ophthalmic composition comprising Indigo Carmine into the cataract extracted eye; and
iv) surgically treating the glaucoma of the Indigo Carmine instilled eye.

In another aspect, provided herein is a method of cataract extraction and treatment of glaucoma in an eye of a patient in need thereof, comprising:
i) instilling an ophthalmic composition comprising Indigo Carmine into the patient's eye;
ii) surgically extracting the cataract of the instilled eye; and
iii) surgically treating the glaucoma of the cataract extracted eye.

In another aspect, provided herein is a method of cataract extraction and treatment of glaucoma in an eye of a patient in need thereof, comprising:
i) instilling an ophthalmic composition comprising Indigo Carmine and Trypan Blue into the patient's eye;
ii) surgically extracting the cataract of the instilled eye; and
iii) surgically treating the glaucoma of the cataract extracted eye.

In another aspect, provided herein is a method of cataract extraction and treatment of glaucoma in an eye of a patient in need thereof, comprising:
i) instilling an ophthalmic composition comprising Indigo Carmine and an ophthalmic composition comprising Trypan Blue into the patient's eye;
ii) surgically extracting the cataract of the instilled eye; and
iii) surgically treating the glaucoma of the cataract extracted eye.

In certain embodiments of the ophthalmic composition, or the method of using the same, as disclosed herein, the ophthalmic composition is an aqueous composition.

In certain embodiments of the ophthalmic composition, or the method of using the same, as disclosed herein, the Indigo Carmine is present in an amount in the range of between approximately 0.001-0.4 wt. %, relative to the ophthalmic composition.

In certain embodiments of the ophthalmic composition, the ophthalmic device, or the method of treating, disclosed herein, the ophthalmic composition further comprises Trypan Blue.

In certain embodiments of the ophthalmic composition, the ophthalmic device, or the method of treating, disclosed herein, the Trypan Blue is present in an amount in the range of between approximately 0.001-0.1 wt. %, relative to the ophthalmic composition.

In certain embodiments of the ophthalmic composition, the ophthalmic device, or the method of treating, disclosed herein, the Trypan Blue is present in an amount less than 0.1 wt. %, such as less than 0.05 wt. %, relative to the ophthalmic composition.

In certain embodiments of the ophthalmic composition, the ophthalmic device, or the method of treating, disclosed herein, the Indigo Carmine is present in an amount in the range of between approximately 0.001-0.4 wt. %, and the Trypan Blue is present in an amount in the range of between approximately 0.001-0.1 wt. %, such as between approximately 0.001-0.05 wt. % or between approximately 0.001-0.045 wt. %, relative to the ophthalmic composition.

In certain embodiments of the ophthalmic composition, the ophthalmic device, or the method of treating, disclosed herein, the ophthalmic composition further comprises another dye, such as Brilliant Blue, Patent Blue, Indocyanine Green, or Fluorescein

In certain embodiments of the ophthalmic composition, the ophthalmic device, or the method of treating, disclosed herein, the ophthalmic composition is an injectable ophthalmic formulation.

In certain embodiments of the ophthalmic composition, the ophthalmic device, or the method of treating, disclosed herein, the ophthalmic composition further comprises one or more additional ophthalmically acceptable excipients and additives.

In certain embodiments of the ophthalmic composition, the ophthalmic device, or the method of treating, disclosed herein, the ophthalmic composition is used for application to an eye, such as via topical application or injection, for example, via injection into the anterior chamber of said eye.

In certain embodiments of the ophthalmic composition, the ophthalmic device, or the method of treating, disclosed herein, the eye is a glaucomatous eye and/or has a cataract.

In certain embodiments of the ophthalmic composition, the ophthalmic device, or the method of treating, disclosed herein, the method includes an ocular surgery, or the ocular surgery is, selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique.

In certain embodiments of the ophthalmic composition, the ophthalmic device, or the method of treating, disclosed herein, the method, or the ocular surgery, includes a combination of two or more of the following ocular surgeries selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique.

In certain embodiments of the ophthalmic composition, the ophthalmic device, or the method of treating, disclosed herein, the ocular surgery is glaucoma surgery, such as a minimally invasive glaucoma surgery (MIGS).

In certain embodiments of the ophthalmic composition, the ophthalmic device, or the method of treating, disclosed herein, the ocular surgery is a combination of glaucoma surgery and cataract surgery.

In certain embodiments of the ophthalmic composition, the ophthalmic device, or the method of treating, disclosed herein, the ocular surgery is a combination of minimally invasive glaucoma surgery (MIGS) and cataract surgery.

In certain embodiments of the ophthalmic composition, the ophthalmic device, or the method of treating, disclosed herein, the ocular surgery is a combination of minimally invasive glaucoma surgery (MIGS) and endothelial keratoplasty.

In certain embodiments of the ophthalmic composition, the ophthalmic device, or the method of treating, disclosed herein, the ocular surgery is a combination of endothelial keratoplasty and cataract surgery.

In certain embodiments of the ophthalmic composition, the ophthalmic device, or the method of treating, disclosed herein, the ophthalmic composition is instilled into the eye by a plurality of injections, such as instilled into the eye by a plurality of injections into the anterior chamber of the eye.

In certain embodiments of the ophthalmic composition, the ophthalmic device, or the method of treating, disclosed herein, the method identifies, marks, or stains an intraocular structure(s) or membrane(s) within the patient's eye in a visually identifiable manner, such in a visually identifiable manner easily visible by the naked eye of a surgeon.

In certain embodiments of the ophthalmic composition, the ophthalmic device, or the method of treating, disclosed herein, the identified, marked, or stained intraocular structure(s) or membrane(s) within the patient's eye is selected from a group consisting of: a fine vessel, an aqueous vein, an episcleral vein, a collector channel, a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

In certain embodiments of the ophthalmic composition, the ophthalmic device, or the method of treating, disclosed herein, the Indigo Carmine of the ophthalmic composition identifies, marks, or stains a trabecular meshwork and the canal of Schlemm in the patient's eye.

In certain embodiments of the ophthalmic composition, the ophthalmic device, or the method of treating, disclosed herein, the Indigo Carmine of the ophthalmic composition identifies, marks, or stains the canal of Schlemm more than a trabecular meshwork in the patient's eye.

In certain embodiments of the ophthalmic composition, the ophthalmic device, or the method of treating, disclosed herein, the Indigo Carmine of the ophthalmic composition identifies, marks, or stains the trabecular meshwork less than a canal of Schlemm in the patient's eye.

In certain embodiments of the ophthalmic composition, the ophthalmic device, or the method of treating, disclosed herein, the method facilitates diagnosis of the patient's eye, facilitates diagnosis of the intraocular structure(s) or membrane(s) within the patient's eye, facilitates an ocular surgeon's diagnosis of fluid flow and drainage of the patient's eye during the ocular surgery, facilitates treatment of the patient's eye, facilitates surgical treatment of the patient's eye, facilitates surgical treatment of the identified intraocular structure(s) or membrane(s) within the eye, and/or facilitates surgical removal of the identified intraocular structure(s) or membrane(s) within the eye.

In certain embodiments of the ophthalmic composition, the ophthalmic device, or the method of treating, disclosed herein, the method facilitates extracting a cataract and treating glaucoma.

In certain embodiments of the ophthalmic composition, the ophthalmic device, or the method of treating, disclosed herein, the instilled ophthalmic composition facilitates accurate and/or precise inserting, placement, positioning, repositioning, lifting, and/or removal, of an ophthalmic device within the patient's eye, such as proximate the identified intraocular structure(s) or membrane(s) within the patient's eye.

In certain embodiments of the ophthalmic composition, the ophthalmic device, or the method of treating, disclosed herein, the ophthalmic device is a stent.

In certain embodiments of the ophthalmic composition, the ophthalmic device, or the method of treating, disclosed herein, the method facilitates an ocular surgeon's determination of the type of stent to utilize during the ocular surgery and/or facilitates an ocular surgeon's placement of the stent during the ocular surgery.

In certain embodiments of the ophthalmic composition, the ophthalmic device, or the method of treating, disclosed herein, the ophthalmic device is a stent, such as a glaucoma stent or a suprachoroidal stent.

In certain embodiments of the ophthalmic composition, the ophthalmic device, or the method of treating, disclosed herein, the ophthalmic device is an intraocular lens during cataract surgery.

In certain embodiments of the ophthalmic composition, the ophthalmic device, or the method of treating, disclosed herein, the ophthalmic device is introduced proximate or into the canal of Schlemm of the patient's eye.

In certain embodiments of the ophthalmic composition, the ophthalmic device, or the method of treating, disclosed herein, the ophthalmic device is inserted into the suprachoroidal space of the patient's eye.

In certain embodiments of the ophthalmic composition, the ophthalmic device, or the method of treating, disclosed herein, the ophthalmic device is pre-treated prior to placement, such as pre-treated with Indigo Carmine and/or pre-treated with Trypan Blue.

In certain embodiments of the ophthalmic composition, the ophthalmic device, or the method of treating, disclosed herein, the method further comprises instilling an ophthalmic composition comprising Trypan Blue.

In certain embodiments of the ophthalmic composition, the ophthalmic device, or the method of treating, disclosed herein, the instilled the ophthalmic composition comprises both Indigo Carmine and Trypan Blue.

In certain embodiments of the ophthalmic composition, the ophthalmic device, or the method of treating, disclosed herein, the ophthalmic composition is instilled into the patient's eye over a period of time in the range of between 1 second to 2 minutes, such as over a period of at least 10 seconds, at least 20 seconds, at least 30 seconds, at least 45 seconds, at least 1 minute, or at least 1.5 minutes.

In certain embodiments of the ophthalmic composition, the ophthalmic device, or the method of treating, disclosed herein, the ophthalmic composition is instilled into the patient's eye over a period of time until the composition egresses from one or more channels in the patient's eye.

In certain embodiments of the ophthalmic composition, the ophthalmic device, or the method of treating, disclosed herein, a plurality of the instillations of the ophthalmic composition is conducted over a period of time until at least 25%, at least 50%, at least 75%, at least 90%, or at least 95%, of the canal of Schlemm is visually identifiable.

In certain embodiments of the ophthalmic composition, the ophthalmic device, or the method of treating, disclosed herein, the method results in reduced surgical manipulation, reduced tissue manipulation, and/or less severe adverse side effects, relative to an ocular surgery not using said ophthalmic composition.

Other features and advantages of the subject matter described herein will be apparent from the description and figures, and from the claims.

### BRIEF DESCRIPTION OF THE DRAWINGS

Aspects of the embodiments described herein may be best understood from the following detailed description when read with the accompanying figures.

FIG. 1A is a schematic diagram of outflow pathways of the eye. illustrating locations of the trabecular (conventional) and uveoscleral (unconventional) aqueous humor outflow pathways. Arrow A points to the site of insertion of a suprachoroidal stent.

FIG. 1B is a schematic diagram of a proposed continuum model of outflow pathways of the eye (adapted from Carreon, T., et al., “Aqueous outflow—A continuum from trabecular meshwork to episcleral veins”, Prog. Retin. Eye Res., 2017, 57:108-133). In this schematic diagram is illustrated a magnified, diagrammatic view of the anterior chamber angle, the trabecular meshwork and downstream (distal) pathways as labeled. The various components of the pathway act as a highly integrated organ system to control aqueous humor flow rather than as isolated regions. Reduced or altered mechanotransduction in the trabecular meshwork is due to alteration of soluble mechanosensing molecules or their deposition. At all levels, basement membrane degradation is impaired resulting in lack of generation of pro- and anti-angiogenic molecules and fragments of type IV collagen. Reduced collector channel frequency and/or dimension in the surrounding region of trabecular meshwork are observed. The fine regulation of degraded protein fragments of basement membrane may be involved in regulation of collector channels and distal flow regions.

FIG. 2 is an operating microscope view of a human eye bank eye with Indigo Carmine injected into the anterior chamber via a small corneal incision, with some of the dye exiting the eye through the aqueous veins. The segmental nature of the episcleral vein distribution is observed in this eye from a 74 year older donor.

FIG. 3 is an operating microscope view of a human eye bank eye with Indigo Carmine injected into the anterior chamber via a small corneal incision when the conjunctiva is reflected, deeper vessels are seen, evidence of the deep scleral plexus.

FIG. 4 is an operating microscope view of a human eye bank eye with Indigo Carmine injected into the anterior chamber via a small corneal incision, with some of the dye exiting the eye through the aqueous veins. The human eye bank eye is from a 36 year old donor, in which a more extensive distribution of episcleral veins than in the older donor eye (FIG. 2), can be seen.

FIG. 5 is an operating microscope view of a surgically excised anterior segment of an eye bank eye after the anterior chamber has been perfused with an Indigo Carmine solution. The central cornea has been trephined (black oval area) and the specimen viewed from behind (as if one is standing on the retina and looking forward). The dark circular ring represents heavy staining of Schlemm's canal. The more lightly stained rings anterior and posterior to Schlemm's canal represent the anterior and posterior aspects of the trabecular meshwork. The iris root is seen beyond the posterior trabecular meshwork attachment.

FIG. 6 is an operating microscope view of a human eye bank eye wherein, following identification of episcleral veins by injection of Indigo Carmine into the anterior chamber, it is possible to cannulate and inject the larger of these veins using either a small gauge needle or a retinal cannula (41 gauge).

FIG. 7 is an operating microscope view of a human eye bank eye with Indigo Carmine injected into the anterior chamber via a small corneal incision during a capsulorhexis procedure. The anterior capsule of the lens is lightly stained with Indigo Carmine, enhancing its visibility.

FIG. 8 is a slit lamp view of a human eye wherein an ocular surface squamous neoplastic lesion is delineated after topical application of Indigo Carmine. A blue outline is seen around the perimeter of the white ocular surface squamous neoplastic lesion.

FIG. 9A is an operating microscope view of a porcine eye upon which a Small Incision Lenticule Extraction procedure (SMILE) has been carried out. From this view, circular horizontal plains have been cut in the corneal stroma, a superficial smaller diameter cut and a deeper cut of larger diameter. The cuts join in the corneal periphery, thereby creating a lenticule. The arrows show the edge of these cuts.

FIG. 9B is an operating microscope view of a porcine eye upon which a Small Incision Lenticule Extraction procedure (SMILE) has been carried out. Indigo Carmine has been injected into the lenticular plain. The white arrows indicate the complete circular edge of the lenticule. The red arrow shows the small incision into the lenticular plain through which the lenticule is removed.

FIG. 10 is a schematic diagram of major corneal loading forces in the steady state, including intralamellar cohesive forces, swelling pressure, endothelial pump, epithelial barrier, intraocular pressure (IOP), endothelial barrier, and lamellar tension, which are necessary to maintain corneal transparency.

FIG. 11 is a schematic diagram of an exemplary aqueous humor circulation pathway in an eye.

FIG. 12 is a schematic diagram of Microinvasive Glaucoma Surgery (MIGS) mechanisms of action, excerpted from E. M. Schehlein, et al., (2017): “Microinvasive glaucoma surgery: an evidence-based assessment,” Expert Review of Ophthalmology, DOI: 10.1080/17469899.2017.1335597, which is (including each of the 91 references cited therein) herein incorporated by reference in their entirety. The anatomic sites of action for each microinvasive glaucoma surgery device are depicted: subconjunctival, trabecular, suprachoroidal, or ciliary body ablation.

FIG. 13 is a schematic diagram of a traditional glaucoma drainage procedure—single exit strategy indicated by bold arrow. An exemplar of the aqueous humor flow pathway is reduced or stopped (indicated by X mark) as a result of including only a single exit (or single exit direction).

FIG. 14 is a schematic diagram of the multiple drainage procedure disclosed herein, which introduces multiple exits for drainage to replicate or approximate normal physiology of the eye. An exemplary aqueous humor flow pattern as a result of the multiple drainage procedure disclosed herein is illustrated both anterior to the lens and along the cornea and through the drainage system (e. g, Trabecular meshwork, Schlemm's canal, interscleral channels (e.g., collector channels).

FIG. 15 is a table listing a set of implants used during micro-invasive glaucoma surgery procedures, excerpted from L. E. Pillunat, et al., (2017): “Micro-invasive glaucoma surgery (MIGS): a review of surgical procedures using stents,” Clinical Ophthalmology, 11:1583-1600, which is (including each of the 55 references cited therein) herein incorporated by reference in their entirety.

FIG. 16 is a table listing a summary and results of select MIGS studies, excerpted from R. Caywood, (Jul. 15, 2018): “MIGS Madness: An Atlas of Options,” https://www.reviewofoptometry.com/article/migs-madness-an-atlas-of-options, which is (including each of the 28 references cited therein) herein incorporated by reference in their entirety.

FIG. 17A is a schematic diagram showing the relative locations of outlet(s) in the anterior chamber according to a generated computer model of the anterior chamber.

FIG. 17B is a schematic diagram showing the position of the outlet location(s) in the anterior chamber according to a generated computer model of the anterior chamber.

FIG. 18 illustrates velocity vectors simulating the flow of the aqueous humour within the anterior chamber as a result of including 1 outlet, according to a generated computer model of the anterior chamber.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Glaucoma is an eye disease in which inappropriate pressure (usually elevated) damages retinal ganglion cells, resulting in permanent loss of field of vision. Left untreated, glaucoma can result in blindness, since the peripheral field of vision is lost initially and care is not typically sought until late in the course of the disease, when the more central field of vision is affected. The present disclosure recognizes the importance of ophthalmic compositions, and methods of using the same, that can facilitate the ocular surgical procedures that may be utilized to treat glaucoma, as well as other diseases and/or conditions of the eye.

Indigo Carmine, also known as 5,5′-indigodisulfonic acid sodium salt or disodium 3,3′-dioxo-2,2′-bi-indolylidene-5,5′-disulfonate, is an acidic, anionic dye (see, e.g., Keng, C. S., et al., “Removal of cationic and anionic dyes by immobilized titanium dioxide loaded activated carbon”, Malays. J. Anal. Sci., 2008, 12:451-457) that is derived from indigo by sulfonation, which renders the compound soluble in water. Indigo Carmine has been approved for use as a food colorant in the US and Europe (has the E number E132). Of critical importance is that Indigo Carmine exhibits low protein binding, which has been attributed to separation of its two sulfonic groups by 8 atoms (see, e.g., Tsopelas, C., et al., “Why certain dyes are useful for localizing the sentinel lymph node”, J. Nucl. Med., 2002, 43:1377-82). Indigo Carmine is associated with a very low rate of both acute and chronic toxicity (see, e.g., Ferber, K. H., “Toxicology of indigo. A review”, J. Environ. Pathol. Toxicol. Oncol., 1987, 7:73-83), and the few adverse reactions reported have been thought to have been idiosyncratic (see, e.g., Amchova, P., et al., “Health safety issues of synthetic food colorants”, Regul. Toxicol. Pharmacol., 2015, 73:914-22). The dye has been used extensively in medicine across a broad range of specialties. For example, in urology, after intravenous injection, it is rapidly filtered by the kidneys and is useful highlighting portions of the urinary tract so that leaks can be detected (see, e.g., Luketic, L., et al., “Options to Evaluate Ureter Patency at Cystoscopy in a World Without Indigo Carmine”, J. Minim. Invasive Gynecol., 2016, 23:878-85). The dye has also been extensively used in lymphatic mapping (see, e.g., Uhara, H., et al., “Sentinel lymph node biopsy in Japan”, Int. J. Clin. Oncol., 2009, 14:490-6), detecting amniotic membrane rupture (see, e.g., Adekola, H., et al., “Outcomes following intra-amniotic instillation with indigo carmine to diagnose prelabor rupture of membranes in singleton pregnancies: a single center experience”, J. Matern. Fetal Neonatal Med., 2016, 29:544-9), cerebrospinal fluid leakage (see, e.g., Kaufman, B., et al., “Acquired spontaneous, nontraumatic normal-pressure cerebrospinal fluid fistulas originating from the middle fossa”, Radiology, 1977, 122:379-87) and to enhance detection of pathology during endoscopy (see, e.g., Brown, S. R., et al., “Chromoscopy versus conventional endoscopy for the detection of polyps in the colon and rectum”, Cochrane Database Syst. Rev., 2016 Apr. 7, 4: CD006439).

The present application provides an ophthalmic composition comprising Indigo Carmine, or an ophthalmic composition comprising a combination of Indigo Carmine and Trypan Blue, for topical or ocular application, such as instillation by injection, and methods of using the same, such as for identification, marking, and/or staining of intraocular structures or membranes, and to facilitate ocular surgeries, such as glaucoma surgery and cataract surgery, among other ocular surgeries disclosed herein.

In certain embodiments, the ophthalmic composition may comprise or consist of a single dye, wherein the single dye is Indigo Carmine, or may comprise or consist of a combination of dyes, wherein the combination of dyes comprises Indigo Carmine and at least one dye selected from the group consisting of: Trypan Blue, Brilliant Blue, Patent Blue, Indocyanine Green, and Fluorescein. In certain embodiments, the combination of dyes is Indigo Carmine and Trypan Blue.

In certain embodiments, the Indigo Carmine contained within the ophthalmic composition disclosed herein may be present in low concentrations, for example, in an amount in the range of between approximately 0.001-0.4 wt. %, relative to the ophthalmic composition, such as present in an amount in the range of between approximately 0.001-0.3 wt. %, between approximately 0.001-0.2 wt. %, between approximately 0.001-0.1 wt. %, between approximately 0.001-0.05 wt. %, between approximately 0.001-0.01 wt. %, between approximately 0.004-0.4 wt. %, between approximately 0.004-0.04 wt. %, between approximately 0.005-0.4 wt. %, between approximately 0.005-0.3 wt. %, between approximately 0.005-0.2 wt. %, between approximately 0.005-0.1 wt. %, between approximately 0.005-0.05 wt. %, between approximately 0.005-0.01 wt. %, between approximately 0.01-0.35 wt. %, between approximately 0.01-0.3 wt. %, between approximately 0.01-0.25 wt. %, between approximately 0.01-0.2 wt. %, between approximately 0.01-0.15 wt. %, between approximately 0.01-0.1 wt. %, between approximately 0.04-0.4 wt. %, between approximately 0.05-0.4 wt. %, between approximately 0.1-0.4 wt. %, between approximately 0.15-0.4 wt. %, between approximately 0.2-0.4 wt. %, between approximately 0.25-0.4 wt. %, between approximately 0.3-0.4 wt. %, between approximately 0.35-0.4 wt. %, between approximately 0.1-0.3 wt. %, between approximately 0.1-0.2 wt. %, between approximately 0.01-0.05 wt. %, or between approximately 0.05-0.1 wt. %, relative to the ophthalmic composition. In certain embodiments, the Indigo Carmine may be present in the ophthalmic composition disclosed herein in an amount of approximately 0.001 wt. %, approximately 0.002 wt. %, approximately 0.003 wt. %, approximately 0.004 wt. %, approximately 0.005 wt. %, approximately 0.006 wt. %, approximately 0.007 wt. %, approximately 0.008 wt. %, approximately 0.009 wt. %, approximately 0.01 wt. %, approximately 0.02 wt. %, approximately 0.03 wt. %, approximately 0.04 wt. %, approximately 0.05 wt. %, approximately 0.06 wt. %, approximately 0.07 wt. %, approximately 0.08 wt. %, approximately 0.09 wt. %, approximately 0.1 wt. %, approximately 0.2 wt. %, approximately 0.3 wt. %, or approximately 0.4 wt. %, relative to the ophthalmic composition. In certain embodiments, the Indigo Carmine may be present in the ophthalmic composition disclosed herein in an amount of at least 0.001 wt. %, at least 0.002 wt. %, at least 0.003 wt. %, at least 0.004 wt. %, at least 0.005 wt. %, at least 0.006 wt. %, at least 0.007 wt. %, at least 0.008 wt. %, at least 0.009 wt. %, at least 0.01 wt. %, at least 0.02 wt. %, at least 0.03 wt. %, at least 0.04 wt. %, at least 0.05 wt. %, at least 0.06 wt. %, at least 0.07 wt. %, at least 0.08 wt. %, at least 0.09 wt. %, at least 0.1 wt. %, at least 0.15 wt. %, at least 0.2 wt. %, at least 0.25 wt. %, at least 0.3 wt. %, or at least 0.35 wt. %, relative to the ophthalmic composition.

In certain embodiments, when the ophthalmic composition comprises Indigo Carmine and Trypan Blue, or when an ophthalmic composition comprising Trypan Blue is co-administered with the ophthalmic composition comprising Indigo Carmine (such as at the same time, or sequentially before or after), the Trypan Blue may be present in an amount in the range of between approximately 0.001-0.1 wt. %, relative to the ophthalmic composition. In certain embodiments, when the ophthalmic composition comprises Indigo Carmine and Trypan Blue, or when an ophthalmic composition comprising Trypan Blue is co-administered with the ophthalmic composition comprising Indigo Carmine (such as at the same time, or sequentially before or after), the Trypan Blue may be present in an amount in the range of at least 0.001 wt. % and less than 0.1 wt. %, relative to the ophthalmic composition, for example, the Trypan Blue is present in an amount in the range of at least 0.001 wt. % and less than 0.05 wt. %, less than 0.04 wt. %, less than 0.03 wt. %, less than 0.02 wt. %, or less than 0.01 wt. %, relative to the ophthalmic composition. In certain embodiments, when the ophthalmic composition comprises Indigo Carmine and Trypan Blue, or when an ophthalmic composition comprising Trypan Blue is co-administered with the ophthalmic composition comprising Indigo Carmine (such as at the same time, or sequentially before or after), the Trypan Blue may be present in an amount in the range of between approximately 0.001-0.1 wt. %, for example, the Trypan Blue may be present in an amount in the range of between approximately 0.001-0.05 wt. %, such as between approximately 0.001-0.045 wt. %, between approximately 0.001-0.04 wt. %, between approximately 0.001-0.035 wt. %, between approximately 0.001-0.03 wt. %, between approximately 0.001-0.025 wt. %, between approximately 0.001-0.02 wt. %, between approximately 0.001-0.015 wt. %, between approximately 0.001-0.01 wt. %, between approximately 0.005-0.1 wt. %, between approximately 0.005-0.05 wt. %, between approximately 0.005-0.045 wt. %, between approximately 0.005-0.04 wt. %, between approximately 0.005-0.035 wt. %, between approximately 0.005-0.03 wt. %, between approximately 0.005-0.025 wt. %, between approximately 0.005-0.02 wt. %, between approximately 0.005-0.015 wt. %, between approximately 0.005-0.01 wt. %, between approximately 0.01-0.1 wt. %, between approximately 0.01-0.05 wt. %, between approximately 0.01-0.045 wt. %, between approximately 0.01-0.04 wt. %, between approximately 0.01-0.035 wt. %, between approximately 0.01-0.03 wt. %, between approximately 0.01-0.025 wt. %, between approximately 0.01-0.02 wt. %, between approximately 0.01-0.015 wt. %, or between approximately 0.02-0.04 wt. %, relative to the ophthalmic composition. In certain embodiments, when the ophthalmic composition comprises Indigo Carmine and Trypan Blue, or when an ophthalmic composition comprising Trypan Blue is co-administered with the ophthalmic composition comprising Indigo Carmine (such as at the same time, or sequentially before or after), the Trypan Blue may be present in an amount of approximately 0.001 wt. %, for example, the Trypan Blue may be present in an amount of approximately 0.005 wt. %, approximately 0.01 wt. %, approximately 0.015 wt. %, approximately 0.02 wt. %, approximately 0.025 wt. %, approximately 0.03 wt. %, approximately 0.035 wt. %, approximately 0.04 wt. %, approximately 0.045 wt. %, approximately 0.05 wt. %, or approximately 0.1 wt. %, relative to the ophthalmic composition.

In certain embodiments, the ophthalmic composition may comprise Indigo Carmine and Trypan Blue, or an ophthalmic composition comprising Indigo Carmine may be co-administered with an ophthalmic composition comprising Trypan Blue (such as at the same time, or sequentially before or after), wherein the Indigo Carmine is present in an amount in the range of between approximately 0.001-0.4 wt. %, and the Trypan Blue is present in an amount in the range of between approximately 0.001-0.1 wt. %, relative to the ophthalmic composition. For example, in certain embodiments, the ophthalmic composition may comprise the Indigo Carmine in an amount in the range of between approximately 0.005-0.3 wt. %, and the Trypan Blue in an amount in the range of between approximately 0.005-0.05 wt. %, relative to the ophthalmic composition, such as comprise the Indigo Carmine in an amount in the range of between approximately 0.005-0.3 wt. %, and the Trypan Blue in an amount in the range of between approximately 0.005-0.045 wt. %, relative to the ophthalmic composition; comprise the Indigo Carmine in an amount in the range of between approximately 0.005-0.3 wt. %, and the Trypan Blue in an amount in the range of between approximately 0.005-0.04 wt. %, relative to the ophthalmic composition; or comprise the Indigo Carmine in an amount of approximately 0.01 wt. %, and the Trypan Blue in an amount of approximately 0.01 wt. %, relative to the ophthalmic composition.

In certain embodiments, the ophthalmic composition disclosed herein is an aqueous composition, such as a sterile aqueous solution. In certain embodiments, the ophthalmic composition disclosed herein comprises or is an ophthalmic irrigation solution, wherein the ophthalmic composition is at physiological pH and comprises an isotonic salt concentration. For example, the ophthalmic irrigation solution may be a balanced salt solution (BSS), a Balanced Salt Solution Plus (BSS Plus 0), an Alsever's salt solution, an Earle's balanced salt solution (EBSS), a Gey's balanced salt solution (GBSS), a Hanks' balanced salt solution (MSS), a Dulbecco's phosphate buffered saline (PBS), a Puck's balanced salt solution, a Ringer's balanced salt solution (RBSS), a Simm's balanced salt solution (SBSS), a TRIS-buffered saline (TBS), or a Tyrode's balanced salt solution solution (TBSS), or combinations thereof. For other examples of irrigation solutions, see, e.g., U.S. Pat. No. 4,550,022, or International Publication WO 1994/008602. In certain embodiments, the ophthalmic composition disclosed herein further comprises one or more additional ophthalmically acceptable excipients and additives. In certain embodiments, the ophthalmic composition disclosed herein further comprises sugar compounds, such as glucose or dextrose. In certain embodiments, the ophthalmic composition disclosed herein further comprises anti-oxidant compounds, such as glutathione. In certain embodiments, the ophthalmic composition disclosed herein is an isotonic, aqueous solution, such as an isotonic, sterile, aqueous solution, having a neutral pH (for example, between pH 6-8, such as between pH 6.5-7.5, between pH 7-7.6, between pH 7.3-7.6, or between pH 6.8-7.2, such as approximately pH 7), and may further comprise certain cations, such as sodium, potassium, calcium, and/or magnesium cations, and comprise certain anions, such as phosphate ion, mono-hydrogen phosphate ion, di-hydrogen phosphate ion, citrate ion, bicarbonate, or chloride ion, or combinations thereof. In certain embodiments, the ophthalmic composition may comprise inorganic salts and/or organic salts, such as sodium chloride, potassium chloride, calcium chloride, magnesium chloride, sodium acetate, sodium phosphate, sodium mono-hydrogen phosphate (sodium mono-hydrogen orthophosphate), sodium di-hydrogen phosphate (sodium di-hydrogen orthophosphate), sodium bicarbonate, or sodium citrate, or combinations thereof. In certain embodiments, the ophthalmic composition disclosed herein has an osmolality between 200-400 mosmol/kg, such as 250-350 mosmol/kg, 300-350 mosmol/kg, or 250-325 mosmol/kg, for example, 200 mosmol/kg, 250 mosmol/kg, 275 mosmol/kg, 300 mosmol/kg, or 325 mosmol/kg, such as 300 mosmol/kg. In certain embodiments, the ophthalmic composition comprising or consisting of Indigo Carmine, or Indigo Carmine and Trypan Blue, may further comprise one or more additional ophthalmically acceptable excipients and additives, comprising for example, carriers, stabilizers, osmolarity adjusting agent, a preservative, a buffer agent, or a tonicity adjusting agent, thickeners and other excipients.

In certain embodiments, the ophthalmic composition disclosed herein has a reduced amount of oxygen content, relative to the oxygen content of atmospheric conditions, for example, an oxygen content of less than 20 mol. %, for example, an oxygen content of less than 15 mol. %, such as an oxygen content of less than 10 mol. %, less than 8 mol. %, less than 5 mol. %, less than 4 mol. %, less than 3 mol. %, less than 2.5 mol. %, or less than 1 mol. %, relative to the oxygen content of atmospheric conditions. In certain embodiments, the ophthalmic composition disclosed herein has a reduced amount of dissolved oxygen content, for example, a dissolved oxygen content of less than 12 mg/L, such as a dissolved oxygen content of less than 10 mg/L, less than 8 mg/L, less than 6 mg/L, less than 5 mg/L, less than 4 mg/L, less than 3 mg/L, less than 2 mg/L, or less than 1 mg/L.

In certain embodiments, the ophthalmic composition disclosed herein is stored in a container, for example a sealed container or sealable container, such as a bottle, a vial, or a syringe. For example, in certain embodiments, the bottle is a glass or plastic bottle, having a volume of 100 mL, 75 mL, 50 mL, 25 mL, 20 mL, 10 mL, 5 mL, or 1 mL. For example, in certain embodiments, the bottle is sealed with a syringe needle piercable cap. For example, in certain embodiments, the vial is a glass or plastic vial, having a volume of 100 mL, 75 mL, 50 mL, 25 mL, 20 mL, 10 mL, 5 mL, or 1 mL. For example, in certain embodiments, the vial is sealed with a syringe needle piercable cap. For example, in certain embodiments, the syringe is a 20 mL syringe, 10 mL syringe, 5 mL syringe, 4 mL syringe, 3 mL syringe, 2 mL syringe, 2.5 mL syringe, 1 mL syringe, or 0.5 mL syringe. For example, in certain embodiments, the syringe containing the ophthalmic composition disclosed herein is vacuum sealed to limit or avoid oxygen exposure to said composition. In certain embodiments, the oxygen content of the head space within the container when storing the ophthalmic composition disclosed herein is a reduced oxygen content, relative to the oxygen content of atmospheric conditions. In certain embodiments, the ophthalmic composition disclosed herein is purged with an inert gas, such as nitrogen or argon gas, prior to sealing within or enclosing within the container. For example, in certain embodiments, herein is purged with an inert gas, such as nitrogen or argon gas, prior to sealing within or enclosing within the container such that the resulting dissolved oxygen content of the purged ophthalmic composition stored within the container upon sealing is less than 12 mg/L, such as less than 10 mg/L, less than 8 mg/L, less than 6 mg/L, less than 5 mg/L, less than 4 mg/L, less than 3 mg/L, less than 2 mg/L, or less than 1 mg/L. In certain embodiments, the head space within the container above the ophthalmic composition disclosed herein is purged with an inert gas, such as nitrogen or argon gas, to limit the oxygen content within the head space of the container storing the ophthalmic composition.

In certain embodiments, the head space within the container above the ophthalmic composition is purged with an inert gas, such as nitrogen or argon gas, to limit the oxygen content of the head space of the container storing said ophthalmic composition. For example, in certain embodiments, the head space within the container above the ophthalmic composition disclosed herein is purged with an inert gas, such as nitrogen or argon gas, to limit the oxygen content within the head space of the container storing the ophthalmic composition to an oxygen content of less than 20 mol. %, for example, purged with an inert gas to limit the oxygen content within the head space to an oxygen content of less than 15 mol. %, such as an oxygen content of less than 10 mol. %, less than 8 mol. %, less than 5 mol. %, less than 4 mol. %, less than 3 mol. %, less than 2.5 mol. %, or less than 1 mol. %, or to less than 12 mg oxygen gas/L inert gas, such as less than 10 mg oxygen gas/L inert gas, less than 8 mg oxygen gas/L inert gas, less than 6 mg oxygen gas/L inert gas, less than 5 mg oxygen gas/L inert gas, less than 4 mg oxygen gas/L inert gas, less than 3 mg oxygen gas/L inert gas, less than 2 mg oxygen gas/L inert gas, or less than 1 mg oxygen gas/L inert gas, or to less than 12 mL oxygen gas/L inert gas, such as less than 10 mL oxygen gas/L inert gas, less than 8 mL oxygen gas/L inert gas, less than 6 mL oxygen gas/L inert gas, less than 5 mL oxygen gas/L inert gas, less than 4 mL oxygen gas/L inert gas, less than 3 mL oxygen gas/L inert gas, less than 2 mL oxygen gas/L inert gas, or less than 1 mL oxygen gas/L inert gas.

In certain embodiments, the head space within the container above the ophthalmic composition and the ophthalmic composition stored therein are purged with an inert gas, such as nitrogen or argon gas, to limit the oxygen content of both the ophthalmic composition and the head space of the container storing said ophthalmic composition. For example, in certain embodiments, the head space within the container above the ophthalmic composition and the ophthalmic composition disclosed herein that is stored in the container are purged with an inert gas, such as nitrogen or argon gas, prior to sealing or enclosing the ophthalmic composition within said container such that the resulting dissolved oxygen content of the purged ophthalmic composition while stored within the sealed or enclosed container is less than 12 mg/L, such as less than 10 mg/L, less than 8 mg/L, less than 6 mg/L, less than 5 mg/L, less than 4 mg/L, less than 3 mg/L, less than 2 mg/L, or less than 1 mg/L.

In certain embodiments, the ophthalmic composition disclosed herein is suitable for application to an eye, for example, is suitable for instillation into the eye of patient in need thereof, such as instillation by injection or via topical application to said eye. In certain embodiments, the ophthalmic composition disclosed herein is an injectable ophthalmic composition. In certain embodiments, the ophthalmic composition disclosed herein is instilled by injection into eye, such as instilled by injection into the anterior chamber of said eye, for example, instilled by a plurality of injections into the anterior chamber of said eye.

In certain embodiments, the patient's eye has one or more ocular conditions or diseases. For example, the patient's eye may be a glaucomatous eye, and/or may have a cataract. In certain embodiments, the patient's eye requires ocular surgery. In certain embodiments, a method for ocular surgery in a patient in need thereof is provided, wherein said method comprises instilling the ophthalmic composition as disclosed herein. For example, in certain embodiments, the method includes an ocular surgery, or the ocular surgery is, selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique. In certain embodiments, the method, or the ocular surgery, includes a combination of two or more of the following ocular surgeries selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique. For example, the ocular surgery is or includes glaucoma surgery, such as minimally invasive glaucoma surgery (MIGS). In certain embodiments, the ocular surgery is or includes cataract surgery. In certain embodiments, the ocular surgery comprises extracting a cataract and treating glaucoma. In certain embodiments, the ocular surgery is or includes endothelial keratoplasty, such as Descemet's Membrane Endothelial Keratoplasty (DMEK). In certain embodiments, the ocular surgery is or includes capsulorhexis. In certain embodiments, the ocular surgery is or includes a small incision lenticule extraction (SMILE). In certain embodiments, the ocular surgery is or includes an Ab externo Canaloplasty (ABeC). In certain embodiments, the ocular surgery is a combination of glaucoma surgery and cataract surgery. In certain embodiments, the ocular surgery is a combination of minimally invasive glaucoma surgery (MIGS) and cataract surgery. In certain embodiments, the ocular surgery is a combination of minimally invasive glaucoma surgery (MIGS) and endothelial keratoplasty. In certain embodiments, the ocular surgery is a combination of endothelial keratoplasty and cataract surgery.

In certain embodiments, a patient for whom the ophthalmic composition disclosed herein, and methods of using the same, may be suitable for include, but are not limited to, a patient that has both cataract and glaucoma and is receiving topical medication to manage the glaucoma; an elderly patient, such as over the age of 40, over 50, over 60, over 70, or over 80 years of age; a patient that has an intraocular pressure control that is suboptimal, such as in an elderly patient; a patient, such as an elderly patient, that has dry eye syndrome. Dry eye syndrome is more common in elderly patient, and topical medication can exacerbate this condition, causing discomfort and reduced vision. Topical anti-glaucoma medications (such as beta blockers) can be absorbed systemically and can have significant (even fatal) side effects. The aim in treating such a patient is to restore sight by removing the cataract and to make the patient independent of topical drug use by surgically reducing eye pressure. As in a routine cataract extraction, a peripheral corneal incision may be made to enter the anterior chamber of the eye and the dye composition may then be instilled, initially to stain the anterior capsule of the cataractous lens—this can facilitate creation of an opening in the capsule (capsulorhexis) in order to gain access to the cataract, which may be removed by phacoemulsification. Following the phacoemulsification, an intraocular lens may be inserted to replace the dioptric power of the cataractous lens that has been removed. Following insertion of the intraocular lens, attention may then be turned to dealing with the glaucoma in the patient's eye. The ophthalmic composition containing Indigo Carmine, as disclosed herein, may be re-injected into the anterior chamber with the aim of delineating critical anatomical structures in the anterior chamber angle, such as Schlemm's canal. Furthermore, injection of the ophthalmic composition containing Indigo Carmine will provide information regarding the presence, location and numbers of collector channels that may exit the eye. This information can then be used to determine the type and location of the MIGS stent to be used to treat the glaucoma. For example, if collector channels are numerous in a particular quadrant of the eye, such as in two quadrants, then stents may be placed in the canal of Schlemm, underlying this area(s) of collector channel preponderance. If, for example, the collector channels are sparse, then a suprachoroidal stent may be used, thereby bypassing this path of resistance. Following insertion of the stent(s), the ophthalmic composition containing Indigo Carmine may be re-instilled to check patency of the stent and flow from the stent out into the aqueous veins or suprachoroidal space. In certain embodiments, the ophthalmic composition may contain Indigo Carmine and Trypan Blue, such as 0.001-0.4 wt. % of Indigo Carmine, for example, 0.1 wt. % of Indigo Carmine, and 0.001-0.1 wt. % of Trypan Blue, for example, 0.0125 wt. % of Trypan Blue, relative to the ophthalmic composition.

In certain embodiments, the methods as disclosed herein identifies, marks, or stains an intraocular structure(s) or membrane(s) within the patient's eye in a visually identifiable manner, for example, the method identifies, marks, or stains an intraocular structure(s) or membrane(s) within the patient's eye in a visually identifiable manner easily visible by the naked eye of a surgeon, and as a result, the method facilitates ready identification of the intraocular structure(s) or membrane(s) within the instilled eye. The method, in certain embodiments, may identify, mark, or stain, an intraocular structure(s) or membrane(s), or a plurality of intraocular structures or membranes, within the patient's eye in a visually identifiable manner. In certain embodiments, the ophthalmic composition, or the method, as disclosed herein, identifies, marks, or stains a portion of the intraocular structure(s) or membrane(s) within the patient's eye, or identifies, marks, or stains a plurality of the intraocular structures or membranes within the patient's eye.

In certain embodiments, the intraocular structure(s) or membrane(s) (or portions thereof) identified, marked, or stained, within the patient's eye by the ophthalmic composition, or the method, as disclosed herein, may be selected from a group consisting of: a fine vessel, an aqueous vein, an episcleral vein, a collector channel, a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane. In certain embodiments, the intraocular structure(s) or membrane(s) (or portions thereof) identified, marked, or stained, within the patient's eye by the ophthalmic composition, or the method, as disclosed herein, is a fine vessel. In certain embodiments, the identified, marked, or stained, intraocular structure(s) or membrane(s) (or portion thereof) is an aqueous vein. In certain embodiments, the identified, marked, or stained, intraocular structure or membrane (or portion thereof) an episcleral vein. In certain embodiments, the identified, marked, or stained, intraocular structure or membrane (or portion thereof) is a collector channel. In certain embodiments, the identified, marked, or stained, intraocular structure(s) or membrane(s) (or portion thereof) is a collector channel/aqueous/episcleral vein system. In certain embodiments, the identified, marked, or stained, intraocular structure(s) or membrane(s) (or portion thereof) is an aqueous drainage system of said eye, such as a conventional drainage system of said eye. In certain embodiments, the identified, marked, or stained, intraocular structure(s) or membrane(s) (or portion thereof) is a conjunctival venous system, such as a subconjunctival space. In certain embodiments, the identified, marked, or stained, intraocular structure(s) or membrane(s) (or portion thereof) is a deep scleral plexus, such as a deep scleral plexus visually identifiable once the conjunctiva is reflected away. In certain embodiments, the identified, marked, or stained, intraocular structure(s) or membrane(s) (or portion thereof) is a trabecular meshwork, such as a posterior aspect of a trabecular meshwork. In certain embodiments, the identified, marked, or stained, intraocular structure(s) or membrane(s) (or portion thereof) is a canal of Schlemm. In certain embodiments, the identified, marked, or stained, intraocular structure(s) or membrane(s) (or portion thereof) is a suprachoroidal space. In certain embodiments, the identified, marked, or stained, intraocular structure(s) or membrane(s) (or portion thereof) is a scleral spur. In certain embodiments, the identified, marked, or stained, intraocular structure(s) or membrane(s) (or portion thereof) is the anterior capsule of a crystalline lens. In certain embodiments, the identified, marked, or stained, intraocular structure(s) or membrane(s) (or portion thereof) is a trabecular meshwork and a canal of Schlemm in the patient's eye. In certain embodiments, the Indigo Carmine of the ophthalmic composition identifies, marks, or stains a canal of Schlemm more than a trabecular meshwork in the patient's eye. In certain embodiments, the Indigo Carmine of the ophthalmic composition identifies, marks, or stains a trabecular meshwork less than a canal of Schlemm in the patient's eye.

In certain embodiments, the ophthalmic composition, or the method, as disclosed herein, facilitates diagnosis of the patient's eye. For example, the ophthalmic composition, or the method, as disclosed herein, facilitates diagnosis of the intraocular structure(s) or membrane(s) within the patient's eye, facilitates an ocular surgeon's diagnosis of fluid flow and drainage of the patient's eye during the ocular surgery, facilitates treatment of the patient's eye, facilitates surgical treatment of the patient's eye, facilitates surgical treatment of the identified intraocular structure(s) or membrane(s) within the eye, and/or facilitates surgical removal of the identified intraocular structure(s) or membrane(s) within the eye, such as facilitates extracting a cataract and treating glaucoma.

In certain embodiments, the methods, as disclosed herein, further comprises introducing an ophthalmic device into the instilled eye and/or the method facilitates the introducing of an ophthalmic device into the instilled eye, such as the Indigo Carmine instilled patient's eye or the Indigo Carmine and Trypan Blue instilled patient's eye. For example, the ophthalmic composition, or the method, as disclosed herein, facilitates accurate and/or precise inserting, placement, positioning, repositioning, lifting, and/or removal, of an ophthalmic device within the patient's eye, such as proximate the identified intraocular structure(s) or membrane(s) within the patient's eye. For example, in certain embodiments, the ophthalmic composition, or the method, as disclosed herein, facilitates accurate and/or precise inserting, placement, positioning, repositioning, lifting, and/or removal, of an ophthalmic device proximate to, or into, the canal of Schlemm of the patient's eye. For example, in certain embodiments, the ophthalmic composition, or the method, as disclosed herein, facilitates accurate and/or precise inserting, placement, positioning, repositioning, lifting, and/or removal, of an ophthalmic device proximate to, or into, the suprachorodial space of the patient's eye.

In certain embodiments, the ophthalmic composition, or the method, as disclosed herein, facilitates an ocular surgeon's determination of the type of stent to utilize during the ocular surgery, facilitates an ocular surgeon's placement of the stent during the ocular surgery, or facilitates an ocular surgeon's determination of the type of stent to utilize and the placement of the stent during the ocular surgery. In certain embodiments, the ophthalmic device utilized in the methods, as disclosed herein, is a stent, such as a glaucoma stent or a suprachoroidal stent. In certain embodiments, the ophthalmic device may be pre-treated with Indigo Carmine and/or pre-treated with Trypan Blue.

In certain embodiments, the method as disclosed herein, further comprises instilling an ophthalmic composition comprising Trypan Blue. In certain embodiments, the method as disclosed herein, the instilled the ophthalmic composition comprises both Indigo Carmine and Trypan Blue.

In certain embodiments, the method as disclosed herein, intraocular structures or membranes of the instilled eye are identified, marked, or stained, by Indigo Carmine prior to extracting of the cataract. In certain embodiments, the method as disclosed herein, intraocular structures or membranes of the instilled eye are identified, marked, or stained, by both Indigo Carmine and Trypan Blue prior to extracting of the cataract.

In certain embodiments, the method as disclosed herein, surgical treatment of the patient's glaucoma in said Indigo Carmine instilled eye comprises introducing an ophthalmic device into said eye. For example, the method may comprise: a) visually identifying an Indigo Carmine stained canal of Schlemm; and b) introducing an ophthalmic device into the patient's eye proximate the Indigo Carmine stained canal of Schlemm.

In certain embodiments, according to the methods disclosed herein, the Indigo Carmine containing ophthalmic composition and the Trypan Blue containing ophthalmic composition are co-instilled concurrently. In certain embodiments, according to the methods disclosed herein, the Indigo Carmine containing ophthalmic composition and the Trypan Blue containing ophthalmic composition are co-instilled sequentially with instilling of the Indigo Carmine containing ophthalmic composition followed by the Trypan Blue containing ophthalmic composition. In certain embodiments, according to the methods disclosed herein, the Indigo Carmine containing ophthalmic composition and the Trypan Blue containing ophthalmic composition are co-instilled sequentially with instilling of the Trypan Blue containing ophthalmic composition followed by the Indigo Carmine containing ophthalmic composition.

In certain embodiments, the ophthalmic composition may be instilled into the patient's eye over a period of time in the range of between 1 second to 2 minutes, according to the methods as disclosed herein. For example, the ophthalmic composition may be instilled into the patient's eye over a period of time in the range of between 1 sec and 1.5 minutes, such as between 10 seconds and 1 minute, between 15 seconds and 2 minutes, between 30 seconds and 1 minute, or between 45 seconds and 1.5 minutes. For example, the ophthalmic composition may be instilled into the patient's eye over a period of at least 10 seconds, such as at least 20 seconds, at least 30 seconds, at least 45 seconds, at least 1 minute, or at least 1.5 minutes. In certain embodiments, the ophthalmic composition may be instilled into the patient's eye over a period of time until the composition egresses from one or more channels in the patient's eye. In certain embodiments, the ophthalmic composition may be instilled into the patient's eye via a plurality of instillations conducted over a period of time until at least 25% of the canal of Schlemm is visually identifiable, such as at least 50%, at least 75%, at least 90%, or at least 95%, of the canal of Schlemm is visually identifiable.

In certain embodiments, use of the ophthalmic composition according to the methods as disclosed herein, results in reduced surgical manipulation, reduced tissue manipulation, and/or less severe adverse side effects, relative to an ocular surgery not using said ophthalmic composition.

The technique Ab interno canaloplasty (ABiC) has been described as a treatment for glaucoma (see, e.g., U.S. Pat. Nos. 7,699,882; 7,967,772; 8,034,105; 8,172,830; 8,491,549; 8,894,603; 9,095,412; and 9,216,109). In this technique, an illuminated microcatheter is inserted into the anterior chamber via a small corneal incision and provides continual trans-scleral visualization of the catheter location within the canal of Schlemm. After catheterisation, the canal of Schlemm is “viscodilated” with an ophthalmic viscosurgical device. This is thought to break adhesion within Schlemm's canal, stretching the trabecular plates creating microperforations within the inner wall of the trabecular meshwork, thus allowing flow into Schlemm's canal, and separating herniations of the inner wall of the trabecular meshwork into the outer wall collector channels (see, e.g., Khaimi, M. A., “Canaloplasty: A Minimally Invasive and Maximally Effective Glaucoma Treatment”, J. Ophthalmol., 2015, 2015:485065).

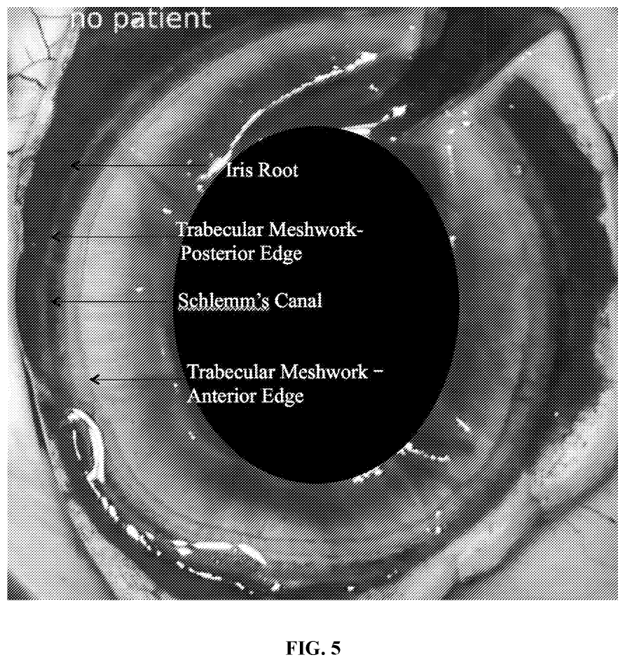

With the utilization of the Indigo Carmine containing ophthalmic compositions disclosed herein, it is possible to identify episcleral/aqueous veins and the deep scleral plexus, thereby allowing for the first time, injection of ophthalmic viscoelastic devices and/or drugs into this distal part of the aqueous drainage system. In certain embodiments, following instillation with an ophthalmic composition containing Indigo Carmine, and with the use of fine needles and cannulas (e.g., 35 gauge needle and 41 gauge cannula), the injection of fluid and ophthalmic viscoelastic devices, in a retrograde fashion, into this system can be accomplished by cannulating the larger aqueous/episcleral veins and slowly injecting in a retrograde fashion (see FIG. 5). This represents a new procedure, herein called Ab Externo Canaloplasty (sometimes referred to herein as ABeC). The Ab Externo Canaloplasty procedure is less invasive than ABiC, since no instruments need to be inserted into the eye—the only intraocular component of the procedure is injection of the ophthalmic composition containing Indigo Carmine via a very small gauge needle into the anterior chamber of the eye. In the ABiC procedure, the presumption is that “stretching” of angle structures is the reason for efficacy, a mechanical hypothesis. However, it may be that the hyaluronan component of the ophthalmic viscosurgical device (OVD) utilized during the ABiC procedure acts to induce VEGF-C (see, e.g., Jung, Y. J., et al., “Hyaluronan-induced VEGF-C promotes fibrosis-induced lymphangiogenesis via Toll-like receptor 4-dependent signal pathway”, Biochem. Biophys. Res. Commun., 2015, 466:339-45). This would allow Ab externo injection of concentrated hyaluronan rather than more viscous and difficult to inject substances. Injection of such solutions could be traced by using dyed solutions (more concentrated than initially used to trace the outflow pathways) to judge how much of the angle has been treated. Other potential drug treatments include injection of VEGF-C (see, e.g., Aspelund, A. et al., “The Schlemm's canal is a VEGF-C/VEGFR-3-responsive lymphatic-like vessel”, J. Clin. Invest., 2014, 124:3975-86) or PROX1 (see, e.g., Park, D. Y., et al., “Lymphatic regulator PROX1 determines Schlemm's canal integrity and identity”, J. Clin. Invest., 2014, 124:3960-74) inducers or appropriate regulating agents.

It has been shown that there are, on average, approximately 13 collector channels in the human eye, while the maximum number of collector channels has been observed as high as 20-30 (see, e.g., Cha, E. D., et al., “Variations in active outflow along the trabecular outflow pathway”, Exp. Eye Res., 2016, 146:354-60). Additionally, the greater concentration of outflow paths in the nasal and inferior quadrants of the eye is further evidence of the asymmetric nature of the distribution of outflow paths, such as collector channels. Accordingly, with the use of the Ab Externo Canaloplasty procedure, it would now be possible to identify each of them intraoperatively and to inject each in a retrograde fashion, thus treating the entire circumference of the drainage angle. Moreover, in the event obstructions exist, as per ABiC, then the Ab Externo Canaloplasty procedure could break them down “segmentally”. In certain embodiments, according to the methods provided herein, an ophthalmic device of sufficiently small inner diameter (or alternatively high flow resistance) could be placed near or adjacent to each collector channel identified by instillation with Indigo Carmine. For example, in certain embodiments, the placement of the ophthalmic device near, proximate, or adjacent to, the identified ocular structure is within a distance of 1-5 inner diameters of said ophthalmic device, such as within 1-2 inner diameters of said ophthalmic device.

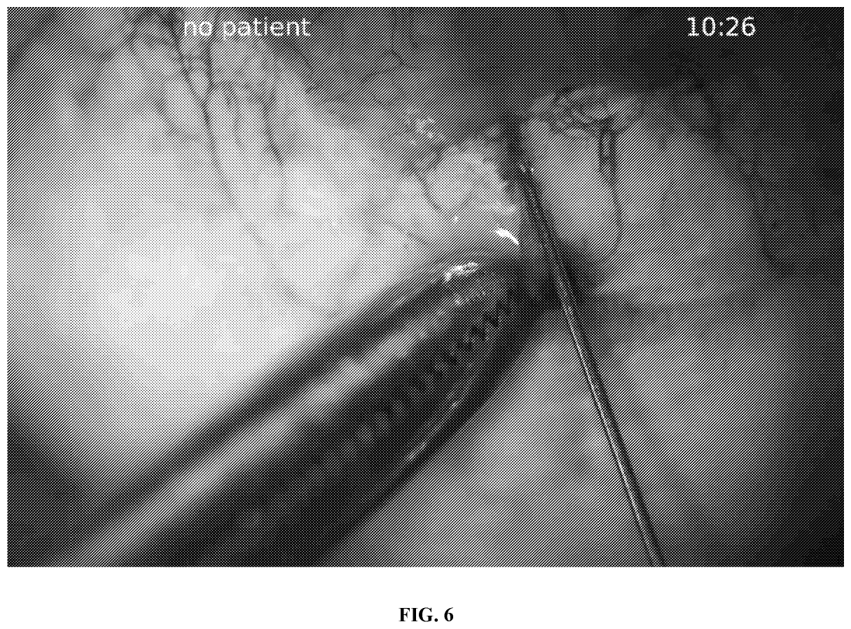

The ophthalmic compositions containing Indigo Carmine also stain the anterior capsule of the crystalline lens, thereby allowing for easier visualization during cataract surgery (see FIG. 6). Importantly, since cataract surgery and glaucoma stent insertion are now frequently performed during the same operation, an ophthalmic composition containing both Indigo Carmine and Trypan Blue would facilitate both surgeries.

In certain embodiments, when Indigo Carmine is combined with other dyes, such as Trypan Blue, the concentration of both dyes can be reduced, thereby limiting any potential toxicity. For example, in certain embodiments, Trypan Blue is usually utilized in concentrations of 0.1-0.06 wt. %, and staining can occur with doses as low as 0.0125 wt. % (see, e.g., Yetik, H., et al., “Determining the lowest trypan blue concentration that satisfactorily stains the anterior capsule”, J. Cataract Refract. Surg., 2002, 28:988-91). When combined with Indigo Carmine, the dosage of Trypan Blue can be further reduced to as little as 0.001 wt. %. For staining the cornea, the angle or other membranes in or on the eye (such as lens capsule or retinal membranes or corneal endothelial/Descemet's membrane preparations as used in endothelial keratoplasty, a form of lamellar corneal transplantation), the dosage of Indigo Carmine can also be reduced. In this way, the risk of toxicity from either dye can be minimized.

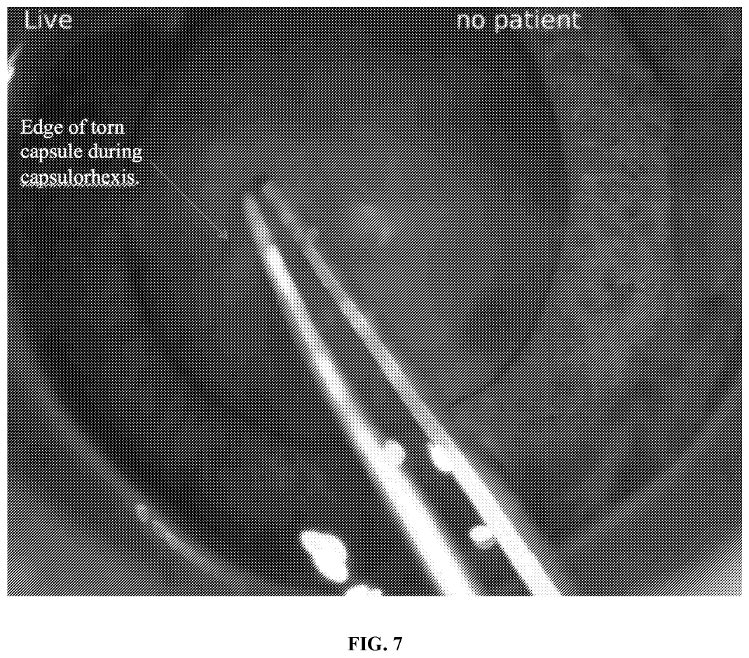

In certain embodiments, the dye delineates eye surface dysplasia and malignancy (ocular surface squamous neoplasia) as has been found in the gut (see, e.g., Oyama, T., “Diagnostic strategies of superficial Barrett's esophageal cancer for endoscopic submucosal dissection”, Dig. Endosc., 2013, 25 Suppl 1:7-12) (see FIG. 7)

In certain embodiments, the dye dissolves in the tear film and can be used for assessing tear film stability (in diseases such as dry eye syndrome) and epithelial loss of the cornea (punctate epithelial erosions) for which dyes such as fluorescein or lissamine green are currently used.

Similarly, in certain embodiments, when the endothelial (posterior layer) of the cornea fails, this layer can be replaced from donor material, introduced into the eye through a small incision in the procedure of endothelial keraroplasty. The disc of tissue to be transplanted is transparent and difficult to see once inserted into the eye. For this reason it is typically stained with a dye, Trypan Blue. However, a combined dye method, utilizing an ophthalmic composition comprising Indigo Carmine and Trypan Blue may be safer and provide better visualization than just by using Trypan Blue alone. Since endothelial keraroplasty can be carried out following cataract surgery, utilizing an ophthalmic composition comprising Indigo Carmine and Trypan Blue, which facilitates both surgeries, would be advantageous.

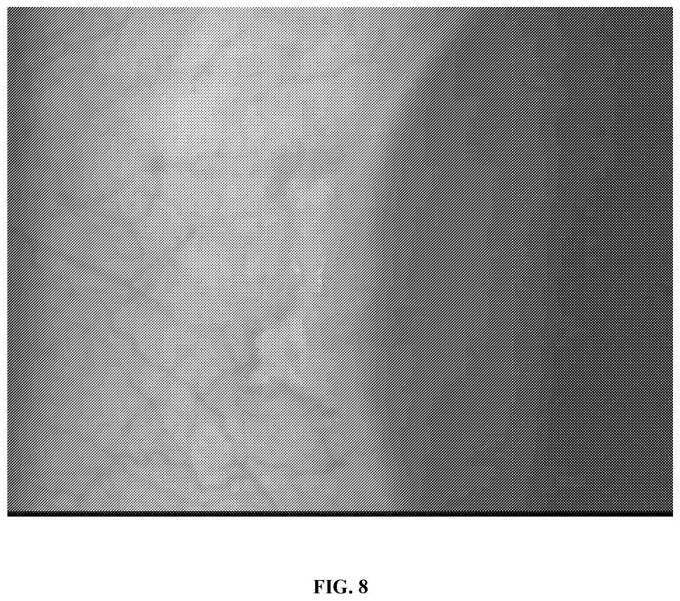

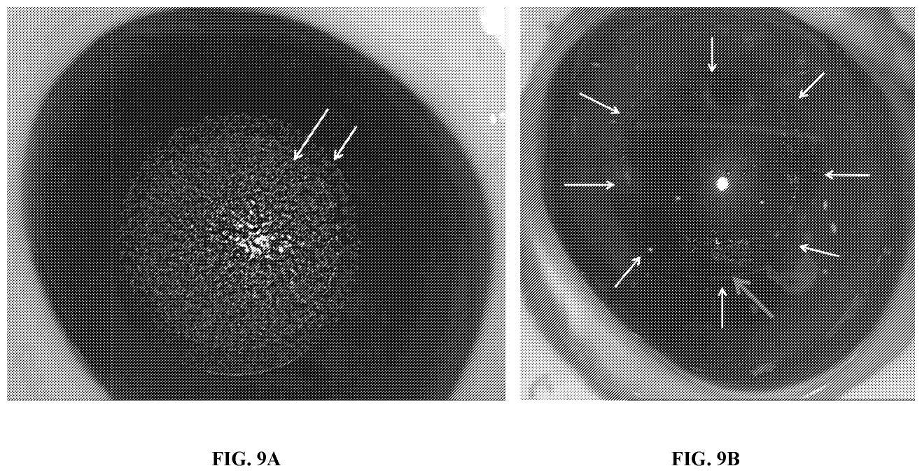

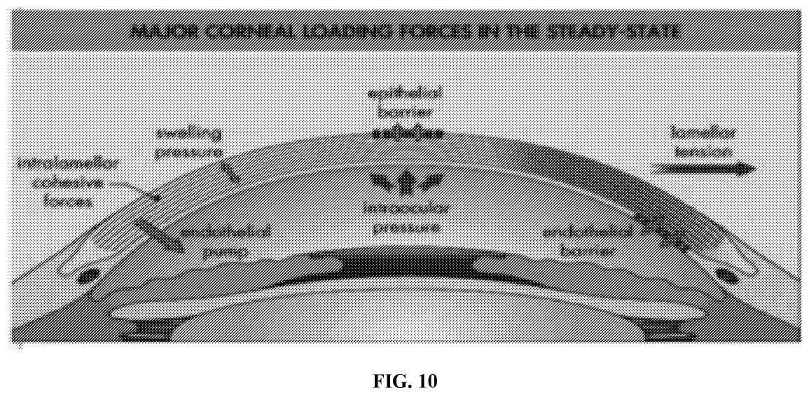

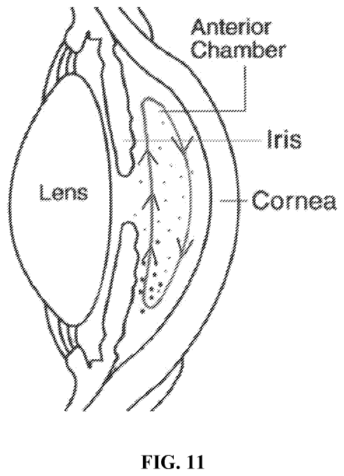

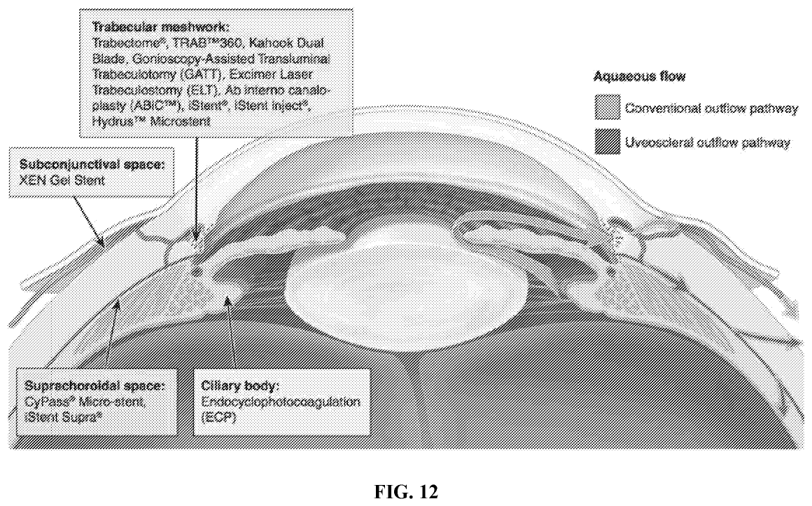

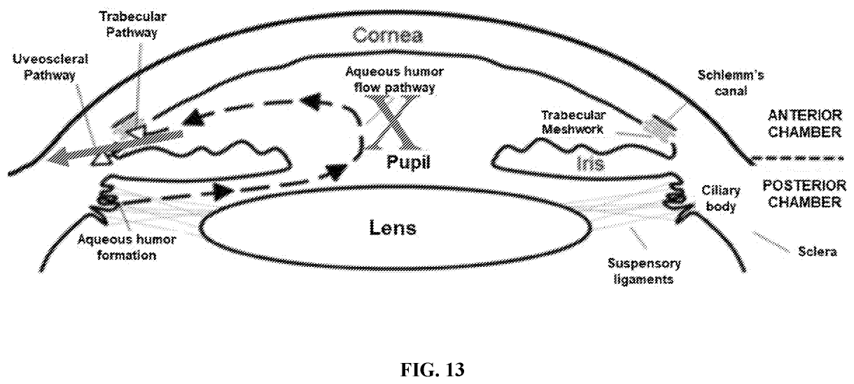

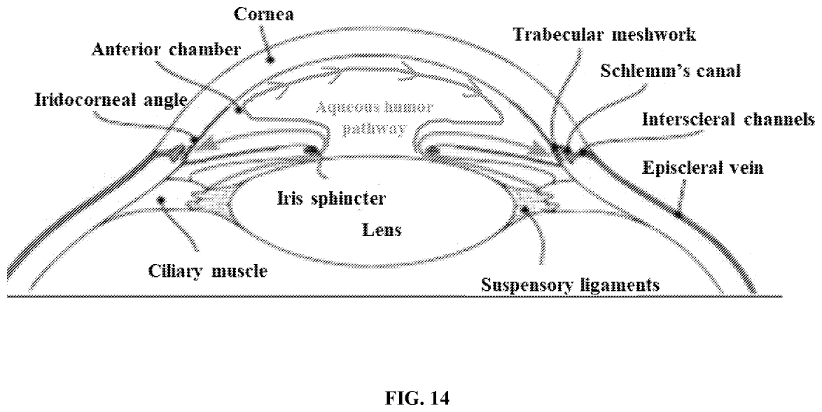

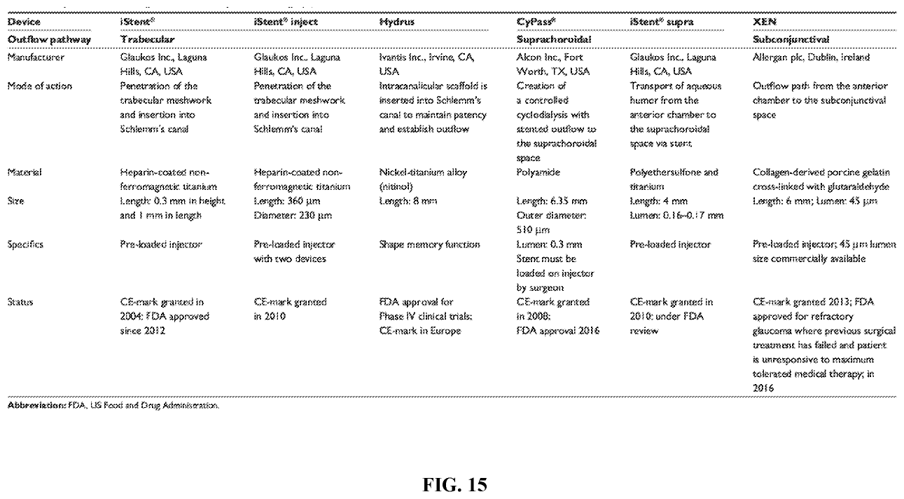

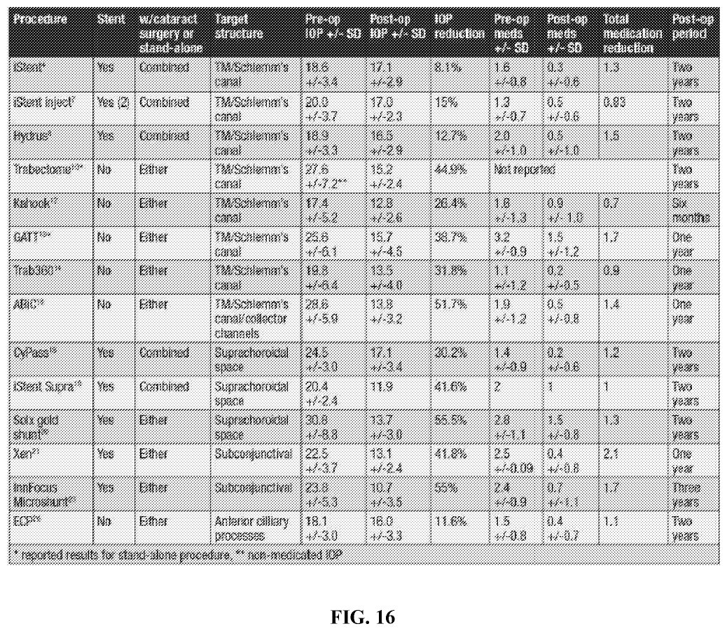

Minimally invasive corneal procedures for correcting refractive error (see, e.g., Titiyal, J. S., et al., “Learning Curve of Small Incision Lenticule Extraction: Challenges and Complications”, Cornea, 2017, 36:1377-1382) have also been developed in recent years. For example, in Small Incision Lenticule Extraction (SMILE), a lenticule of corneal stroma is created within the stroma using a laser and then removed through a small incision. Occasionally, the cuts that create the lenticule are discontinuous, making lenticule extraction difficult. This can result in damage to the corneal stroma in attempts to remove the lenticule, inability to remove the lenticule or incomplete removal of the lenticule, resulting in an irregular cornea, consequent astigmatism and poor vision. Since the cornea is transparent, identifying the lenticule after laser incisions have been made is problematic. By injecting an ophthalmic composition comprising Indigo Carmine into the cornea, for example, along the laser incision planes, it is possible to identify and determine the extent of these incisions, particularly the rounded edge of the lenticule (see FIG. 8), thereby identifying areas of incision discontinuity. These areas can then be manually separated so that either an intact lenticule can be removed or a missing piece of lenticule can be identified and removed separately. The relatively reduced protein binding of Indigo Carmine (compared to other ophthalmic dyes), and that it generally does not diffuse through tissues, makes it ideal for use during SMILE procedures.

The cornea is avascular, and therefore relies largely on circulation of aqueous humor in the anterior chamber of the eye for its nutrition. For this to occur efficiently the aqueous humor circulates in the anterior chamber to provide relatively uniform “coverage” of the posterior cornea. Without being bound by theory, one mechanism by which this circulation occurs is from convection currents that result because the cornea is cooler (by about 2° C.) than aqueous humor (which is at body temperature) that enters the anterior chamber. While egress of the aqueous humor from the anterior is not uniform through the 360° of the angle of the eye, the arrangement of the collector channel low resistance pathways is compatible with maintaining these currents and relatively uniform nutrition of the cornea.

A lack of adequate, uniform corneal nutrition can lead to, and eventually cause, malfunction of a monolayer of cells on the posterior cornea (the corneal endothelium), and this result can be measured by counting endothelial cell numbers and/or measuring the overlying corneal thickness. A lack of adequate, uniform corneal nutrition may result from inadequate or diminished aqueous humor circulation, relative to a healthy subject.

It is noted that during sleep, as the closed eyelid warms the cornea, these aqueous humor currents resulting from convection are abolished. However, rapid eye movement during sleep causes a stirring of the aqueous humor, thereby maintaining corneal nutrition (Maurice D M., “The Von Sallmann Lecture 1996: an ophthalmological explanation of REM sleep,” Exp Eye Res. (1998 February), 66(2):139-45).

In most forms of glaucoma drainage surgery, the overriding aim is to lower intraocular pressure (IOP). Such surgeries do not give consideration to the effects the surgery may have on the aqueous circulation and/or corneal nutrition. However, inadequate drainage and circulation of the aqueous humor can lead to a disruption of the circulation, which may initially result in sectorial endothelial malfunction and overlying corneal edema, before more severe long-term effects are recognized.

Long-term data on the effects of trabeculetomy on endothelial cell loss have been reported (Okumura N, Matsumoto D, Okazaki Y, Koizumi N, Sotozono C, Kinoshita S, Mori K.; “Wide-field contact specular microscopy analysis of corneal endothelium post trabeculectomy,” Graefes Arch Clin Exp Ophthalmol. 2018 April; 256(4):751-757), and show that corneal endothelial cell loss was greatest in the central cornea and near the drainage site (50% and 65% cell loss respectively at 4 years), possibly due to aqueous stagnation. Additionally, in 2018, the CyPass Stent was initially voluntarily withdrawn from the market and then the subject of a Class 1 recall by the FDA. The reason for the recall was that at five-year follow-up, 27% of implanted patients had more than 30% loss in endothelial cell density, which was attributed to mechanical issues with the stent, yet in trabeculectomy, no stent is used, yet the endothelial loss is apparently greater. (https://www.fda.gov/MedicalDevices/Safety/AlertsandNotices/ucm624283.htm).

According to the methods disclosed herein, the method includes introducing a plurality of stents into an eye of a subject, following instillation with Indigo Carmine (for example, introducing a minimum of two stents, such as introducing a minimum of three stents, into the eye of a subject, following instillation with Indigo Carmine) to maintain a level of aqueous humor circulation within the subject's eye approximating a healthy level of aqueous humor circulation, such as level of aqueous humor circulation at physiological conditions. In certain embodiments, the level of aqueous humor circulation achieved according to the methods disclosed herein avoids the side effects that result from utilizing a single drainage pathway, such as a single drainage pathway introduced in the anterior eye (e.g., as is performed in a trabeculectomy or a CyPass procedure). The side effects that result from utilizing a single drainage pathway, such as a single drainage pathway introduced in the anterior eye significantly reduces or completely stops the thermal aqueous humor circulation in the anterior eye.

In certain embodiments, the methods provided herein include instilling Indigo Carmine, alone or in combination with another dye, such as Trypan Blue (e.g., concurrently, sequentially, or as single mixture), into the patient's eye to identify ocular drainage structures. The instillation of the Indigo Carmine allows for the identification of ocular drainage structures in the eye (e.g., via staining), such as major outflow pathways, the main or larger collector channels in said eye (the main or larger collector channels in said eye are sometimes referred to as “collector channel trunks” in said eye, a term describing the larger collectors channels in said eye exiting the canal of Schlemm via openings known as ostia), the trabecular meshwork, and the canal of Schlemm. The instillation of additional dyes, such as Trypan Blue, with the Indigo Carmine (e.g., concurrently, sequentially, or as single mixture), into the patient's eye, allows for the staining of other ocular structures in addition to the ocular structures identified by said Indigo Carmine, according to the methods provided herein. For example, the instillation of Trypan Blue may allow for the identification (e.g., via staining) of the capsule of the lens, which is be beneficial in the context of an ocular surgery that combines cataract and glaucoma surgery. In certain embodiments, upon identifying the ocular drainage structures, such as main collector channel trunks, an ophthalmic device, such as a stent, may be placed proximate or adjacent to a plurality, or each, of the identified ocular drainage structures, such as main collector channel trunks. In specific embodiments, at least 1 or at least 2 stents of the plurality of stents are placed in, proximate, or adjacent to, 1 or 2 identified ocular drainage structures located approximately opposite a main collector channel. In certain embodiments, a plurality of stents are placed in, proximate, or adjacent to, an equal plurality of the identified ocular drainage structures. In certain embodiments, 2, 3, 4, 5, or 6 stents are placed in, proximate, or adjacent to, 2, 3, 4, 5, or 6 of the identified ocular drainage structures, respectively. In certain embodiments, 3, 4, 5, or 6 stents are placed in, proximate, or adjacent to, 3, 4, 5, or 6 of the identified ocular drainage structures, respectively. In certain embodiments, 2 stents of the plurality of stents are placed in, proximate, or adjacent to, a single identified ocular drainage structure, and the remaining of the plurality of stents (e.g., 1, 2, 3, 4, or 5 stents) are distributed among an equal remaining plurality number of the identified ocular drainage structures. For example, in certain embodiments, one or more stents of the plurality of stents could be placed in the trabecular meshwork, suprachoroidal space or even in, proximate, or adjacent to, the subconjunctival space. In specific embodiments, the identified ocular drainage structure is a collector channel. In specific embodiments, the identified ocular drainage structure is a collector channel trunk. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In specific embodiments, the identified ocular drainage structure is the deep scleral plexus. In specific embodiments, the identified ocular drainage structure is the episcleral/aqueous veins. In certain embodiments, utilization of the method provided herein approximates or replicates the physiological state of that particular eye (e.g., physiological aqueous humor circulation, physiological corneal endothelial health, and physiological corneal health)—providing a type of custom drainage procedure approximating physiological conditions. In this way, the physiological aqueous humor circulation, corneal endothelial health, and corneal health, of said eye would be maintained by a plurality of stent placements positioned and distributed around the circumference of the drainage angle of the patient's eye. In specific embodiments, utilization of the method provided herein approximates or replicates physiological aqueous humor circulation. In specific embodiments, utilization of the method provided herein approximates or replicates physiological corneal endothelial health. In specific embodiments, utilization of the method provided herein approximates or replicates physiological corneal health. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, according to the methods provided herein, the visualization of ocular structures, such as ocular drainage structures, that may be achieved by instillation of Indigo Carmine into the subject's eye may be enhanced by its fluorescence characteristics. For example, in a reduced state, Indigo Carmine becomes fluorescent, with absorption and emission maxima at 436 nm and 528 nm, respectively (McCorquodale E M, Piper J, Colyer C L.; “Capillary electrophoresis with laser-induced fluorescence detection of indigo carmine and indigo carmine-labeled proteins,” J Capill Electrophor Microchip Technol. 2002 September-December; 7(5-6):95-101). In certain embodiments, enhanced visualization and detection of ocular structures, such as ocular drainage structures, may be achieved with instilled Indigo Carmine by detection of the Indigo Carmine fluorescence at the absorption and emission maxima wavelengths (e.g., at 436 nm and/or at 528 nm). In certain embodiments, the methods provided herein include utilizing enhanced visualization and detection of instilled Indigo Carmine (alone or in combination with another dye, such as Trypan Blue) within a patient's eye under adverse conditions, such as corneal opacity, or when viewing the structures within the patient's eye may be difficult.

In certain embodiments, the methods provided herein include introducing an ophthalmic device, such as introducing a plurality of ophthalmic devices, into an eye of a patient in need thereof, said method comprising: i) instilling an ophthalmic composition comprising Indigo Carmine into the patient's eye; and ii) introducing the plurality of ophthalmic devices into the instilled eye, wherein the plurality of ophthalmic devices are positioned around the circumference of the drainage angle to maintain physiological aqueous humor circulation, maintain corneal nutrition, maintain corneal endothelial cell density and/or mitigate corneal endothelial cell loss. In specific embodiments, the ophthalmic device is a stent. In specific embodiments, the plurality of stents are positioned around the circumference of the drainage angle such that each stent is placed in, proximate, or adjacent to, an identified ocular drainage structure. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is a collector channel trunk. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In specific embodiments, the identified ocular drainage structure is the deep scleral plexus. In specific embodiments, the identified ocular drainage structure is an episcleral/aqueous vein. In specific embodiments, the method maintains or approximates physiological aqueous humor circulation. In specific embodiments, the method maintains or approximates physiological corneal endothelial health. In specific embodiments, the method maintains or approximates physiological corneal nutrition. In specific embodiments, the method maintains or approximates physiological corneal endothelial cell density and/or mitigates corneal endothelial cell loss. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the methods provided herein include introducing an ophthalmic device, such as introducing a plurality of ophthalmic devices, into an eye of a patient in need thereof, said method comprising: i) instilling an ophthalmic composition comprising Indigo Carmine and Trypan Blue into the patient's eye; and ii) introducing the plurality of ophthalmic devices into the instilled eye, wherein the plurality of ophthalmic devices are positioned around the circumference of the drainage angle to maintain physiological aqueous humor circulation, maintain corneal nutrition, maintain corneal endothelial cell density and/or mitigate corneal endothelial cell loss. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the methods provided herein include introducing an ophthalmic device, such as introducing a plurality of ophthalmic devices, into an eye of a patient in need thereof, said method comprising i) instilling an ophthalmic composition comprising Indigo Carmine and an ophthalmic composition comprising Trypan Blue into the patient's eye; and ii) introducing the plurality of ophthalmic devices into the instilled eye, wherein the plurality of ophthalmic devices are positioned around the circumference of the drainage angle to maintain physiological aqueous humor circulation, maintain corneal nutrition, maintain corneal endothelial cell density and/or mitigate corneal endothelial cell loss. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the methods provided herein include introducing a plurality of ophthalmic devices into an eye of a patient in need thereof, wherein the plurality of ophthalmic devices are placed in, proximate, or adjacent to, one or more identified ocular drainage structures. In certain embodiments, the plurality of ophthalmic devices are placed in, proximate, or adjacent to, an equal plurality of the identified ocular drainage structures. In certain embodiments, a plurality of stents (e.g., 2, 3, 4, 5, or 6 ophthalmic devices, such as 3, 4, 5, or 6 ophthalmic devices) are distributed around the circumference of the drainage angle and placed in, proximate, or adjacent to, identified ocular drainage structures (e.g., an equal plurality of identified ocular drainage structures, or a less than equal plurality of identified ocular drainage structures) in said instilled eye. In certain embodiments, 2, 3, 4, 5, or 6 ophthalmic devices are placed in, proximate, or adjacent to, 2, 3, 4, 5, or 6 of the identified ocular drainage structures, respectively. In certain embodiments, 3, 4, 5, or 6 ophthalmic devices are placed in, proximate, or adjacent to, 3, 4, 5, or 6 of the identified ocular drainage structures, respectively. In specific embodiments, the ophthalmic device is a stent. In specific embodiments, 2 stents are placed in, proximate, or adjacent to, 2 of the identified ocular drainage structures. In specific embodiments, 2 stents are placed in, proximate, or adjacent to, a single identified ocular drainage structure. In specific embodiments, 3 stents are placed in, proximate, or adjacent to, 3 of the identified ocular drainage structures. In specific embodiments, 3 stents are placed in, proximate, or adjacent to, a single identified ocular drainage structure. In specific embodiments, 4 stents are placed in, proximate, or adjacent to, 4 of the identified ocular drainage structures. In specific embodiments, 5 stents are placed in, proximate, or adjacent to, 5 of the identified ocular drainage structures. In specific embodiments, 6 stents are placed in, proximate, or adjacent to, 6 of the identified ocular drainage structures. In certain embodiments, 2 stents of the plurality of stents are placed in, proximate, or adjacent to, a single identified ocular drainage structure, and the remaining of the plurality of stents (e.g., 1, 2, 3, 4, or 5 stents, such as 1, 2, 3, or 4 stents) are distributed among an equal remaining plurality number of the identified ocular drainage structures. In specific embodiments, 3 stents are placed in, proximate, or adjacent to, a single identified ocular drainage structure, and the remaining stents of the plurality of stents are placed in, proximate, or adjacent to, an equal remaining plurality number of the identified ocular drainage structures. In certain embodiments, 6 stents are placed in, proximate, or adjacent to, identified ocular drainage structures, such that the 6 stents are distributed among 5, 4, 3 or 2, such as 4, 3, or 2, identified ocular drainage structures. In certain embodiments, 5 stents are placed in, proximate, or adjacent to, identified ocular drainage structures, such that the 5 stents are distributed among 4, 3, or 2 identified ocular drainage structures. In certain embodiments, 4 stents are placed in, proximate, or adjacent to, identified ocular drainage structures, such that the 4 stents are distributed among 3 or 2 identified ocular drainage structures. In specific embodiments, the plurality is 3. In specific embodiments, the plurality is 4. In specific embodiments, the plurality is 5. In specific embodiments, the plurality is 6. In specific embodiments, the plurality is 7. In specific embodiments, the plurality is 8. In specific embodiments, the plurality is 9. In specific embodiments, the plurality is 10. In certain embodiments, one or more stents of the plurality of stents could be placed in the trabecular meshwork, suprachoroidal space or even in, proximate, or adjacent to, the subconjunctival space. In specific embodiments, the identified ocular drainage structure is a collector channel. In specific embodiments, the identified ocular drainage structure is a collector channel trunk. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In specific embodiments, the identified ocular drainage structure is the deep scleral plexus. In specific embodiments, the identified ocular drainage structure is the episcleral/aqueous veins. In certain embodiments, utilization of the method provided herein approximates or replicates the physiological state of that particular eye (e.g., physiological aqueous humor circulation, physiological corneal endothelial health, and physiological corneal health)—providing a type of custom drainage procedure approximating physiological conditions. In this way, the physiological aqueous humor circulation, corneal endothelial health, and corneal health, of said eye would be maintained by a plurality of stent placements positioned and distributed around the circumference of the drainage angle of the patient's eye. In specific embodiments, utilization of the method provided herein approximates or replicates physiological aqueous humor circulation. In specific embodiments, utilization of the method provided herein approximates or replicates physiological corneal endothelial health. In specific embodiments, utilization of the method provided herein approximates or replicates physiological corneal health. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein maintains physiological aqueous humor circulation of an eye of a patient following ocular surgery of the patient's eye, comprising: i) instilling an ophthalmic composition comprising Indigo Carmine into the patient's eye; ii) performing the ocular surgery of the patient's instilled eye; and iii) introducing a plurality of ophthalmic devices into the instilled eye, wherein the plurality of ophthalmic devices are positioned around the circumference of the drainage angle.

In certain embodiments, the method provided herein maintains physiological aqueous humor circulation of an eye of a patient following ocular surgery of the patient's eye, comprising: i) instilling an ophthalmic composition comprising Indigo Carmine and Trypan Blue into the patient's eye; ii) performing the ocular surgery of the patient's instilled eye; and iii) introducing a plurality of ophthalmic devices into the instilled eye, wherein the plurality of ophthalmic devices are positioned around the circumference of the drainage angle.

In certain embodiments, the method provided herein maintains physiological aqueous humor circulation of an eye of a patient following ocular surgery of the patient's eye, comprising: i) instilling an ophthalmic composition comprising Indigo Carmine and an ophthalmic composition comprising Trypan Blue into the patient's eye; ii) performing the ocular surgery of the patient's instilled eye; and iii) introducing a plurality of ophthalmic devices into the instilled eye, wherein the plurality of ophthalmic devices are positioned around the circumference of the drainage angle.

In certain embodiments, the method provided herein is for ocular surgery of an eye of a patient in need thereof, comprising: i) instilling an ophthalmic composition comprising Indigo Carmine into the patient's eye; ii) performing the ocular surgery of the patient's instilled eye; and iii) introducing a plurality of ophthalmic devices into the instilled eye, wherein the plurality of ophthalmic devices are positioned in a plurality of directions around the circumference of the drainage angle. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for ocular surgery of an eye of a patient in need thereof, comprising: i) instilling an ophthalmic composition comprising Indigo Carmine and Trypan Blue into the patient's eye; ii) performing the ocular surgery of the patient's instilled eye; and iii) introducing a plurality of ophthalmic devices into the instilled eye, wherein the plurality of ophthalmic devices are positioned in a plurality of directions around the circumference of the drainage angle. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for ocular surgery of an eye of a patient in need thereof, comprising: i) instilling an ophthalmic composition comprising Indigo Carmine and an ophthalmic composition comprising Trypan Blue into the patient's eye; ii) performing the ocular surgery of the patient's instilled eye; and iii) introducing a plurality of ophthalmic devices into the instilled eye, wherein the plurality of ophthalmic devices are positioned in a plurality of directions around the circumference of the drainage angle. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for introducing an ophthalmic device into an eye of a patient in need thereof, comprising: i) instilling an ophthalmic composition comprising Indigo Carmine into the patient's eye; and ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for introducing an ophthalmic device into an eye of a patient in need thereof, comprising: i) instilling an ophthalmic composition comprising Indigo Carmine and Trypan Blue into the patient's eye; and ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for introducing an ophthalmic device into an eye of a patient in need thereof, comprising: i) instilling an ophthalmic composition comprising Indigo Carmine and an ophthalmic composition comprising Trypan Blue into the patient's eye; and ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for glaucoma surgery of an eye of a patient in need thereof, comprising: i) instilling an ophthalmic composition comprising Indigo Carmine into the patient's eye; and ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for glaucoma surgery of an eye of a patient in need thereof, comprising: i) instilling an ophthalmic composition comprising Indigo Carmine and Trypan Blue into the patient's eye; and ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for glaucoma surgery of an eye of a patient in need thereof, comprising: i) instilling an ophthalmic composition comprising Indigo Carmine and an ophthalmic composition comprising Trypan Blue into the patient's eye; and ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for maintaining physiological aqueous humor circulation of an eye of a patient following ocular surgery of the patient's eye, comprising: i) instilling an ophthalmic composition comprising Indigo Carmine into the patient's eye; ii) performing the ocular surgery (e.g., cataract surgery) of the patient's instilled eye; iii) optionally, further instilling the ophthalmic composition comprising Indigo Carmine into the patient's eye following the ocular surgery; and iv) introducing a plurality of ophthalmic devices into the instilled eye, wherein the plurality of ophthalmic devices are positioned around the circumference of the drainage angle.

In certain embodiments, the method provided herein is for maintaining physiological aqueous humor circulation of an eye of a patient following ocular surgery of the patient's eye, comprising: i) instilling an ophthalmic composition comprising Indigo Carmine and Trypan Blue into the patient's eye; ii) performing the ocular surgery (e.g., cataract surgery) of the patient's instilled eye; iii) optionally, further instilling the ophthalmic composition comprising Indigo Carmine into the patient's eye following the ocular surgery; and iv) introducing a plurality of ophthalmic devices into the instilled eye, wherein the plurality of ophthalmic devices are positioned around the circumference of the drainage angle.

In certain embodiments, the method provided herein is for maintaining physiological aqueous humor circulation of an eye of a patient following ocular surgery of the patient's eye, comprising: i) instilling an ophthalmic composition comprising Indigo Carmine and an ophthalmic composition comprising Trypan Blue into the patient's eye; ii) performing the ocular surgery (e.g., cataract surgery) of the patient's instilled eye; iii) optionally, further instilling the ophthalmic composition comprising Indigo Carmine into the patient's eye following the ocular surgery; and iv) introducing a plurality of ophthalmic devices into the instilled eye, wherein the plurality of ophthalmic devices are positioned around the circumference of the drainage angle.

In certain embodiments, the method provided herein is for ocular surgery of an eye of a patient in need thereof, comprising: i) instilling an ophthalmic composition comprising Indigo Carmine into the patient's eye; ii) performing the ocular surgery (e.g., cataract surgery) of the patient's instilled eye; iii) optionally, further instilling the ophthalmic composition comprising Indigo Carmine into the patient's eye following the ocular surgery; and iv) introducing a plurality of ophthalmic devices into the instilled eye, wherein the plurality of ophthalmic devices are positioned in a plurality of directions around the circumference of the drainage angle. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for ocular surgery of an eye of a patient in need thereof, comprising: i) instilling an ophthalmic composition comprising Indigo Carmine and Trypan Blue into the patient's eye; ii) performing the ocular surgery (e.g., cataract surgery) of the patient's instilled eye; iii) optionally, further instilling the ophthalmic composition comprising Indigo Carmine into the patient's eye following the ocular surgery; and iv) introducing a plurality of ophthalmic devices into the instilled eye, wherein the plurality of ophthalmic devices are positioned in a plurality of directions around the circumference of the drainage angle. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method provided herein is for ocular surgery of an eye of a patient in need thereof, comprising: i) instilling an ophthalmic composition comprising Indigo Carmine and an ophthalmic composition comprising Trypan Blue into the patient's eye; ii) performing the ocular surgery (e.g., cataract surgery) of the patient's instilled eye; iii) optionally, further instilling the ophthalmic composition comprising Indigo Carmine into the patient's eye following the ocular surgery; and iv) introducing a plurality of ophthalmic devices into the instilled eye, wherein the plurality of ophthalmic devices are positioned in a plurality of directions around the circumference of the drainage angle. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the method disclosed herein facilitates accurate and/or precise inserting, placement, positioning, repositioning, lifting, and/or removal, of a plurality of ophthalmic devices within the Indigo Carmine instilled patient's eye, wherein the plurality of ophthalmic devices are positioned around the circumference of the drainage angle. For example, in certain embodiments, the method facilitates accurate and/or precise inserting, placement, positioning, repositioning, lifting, and/or removal, of a plurality of ophthalmic devices within the Indigo Carmine instilled patient's eye, wherein the plurality of ophthalmic devices are positioned in a plurality of locations around the circumference of the drainage angle. In certain embodiments, the method facilitates accurate and/or precise inserting, placement, positioning, repositioning, lifting, and/or removal, of a plurality of ophthalmic devices within the Indigo Carmine instilled patient's eye, wherein the plurality of ophthalmic devices are positioned in a plurality of locations around the circumference of the drainage angle to form a plurality of drain or flow patterns of the aqueous humor in the instilled patient's eye.

In certain embodiments, the plurality of ophthalmic devices introduced into the instilled eye, according to the method disclosed herein, is at least 2 ophthalmic devices, such as 2-10 or 2-6 ophthalmic devices. In certain embodiments, the plurality of ophthalmic devices introduced into the instilled eye, according to the method disclosed herein, is at least 3 ophthalmic devices, such as 3-10 or 3-6 ophthalmic devices. In certain embodiments, the plurality of ophthalmic devices introduced into the instilled eye, according to the method disclosed herein, is at least 3 ophthalmic devices, at least 4 ophthalmic devices, at least 5 ophthalmic devices, at least 6 ophthalmic devices, at least 7 ophthalmic devices, at least 8 ophthalmic devices, at least 9 ophthalmic devices, at least 10 ophthalmic devices, at least 11 ophthalmic devices, at least 12 ophthalmic devices, at least 13 ophthalmic devices, at least 14 ophthalmic devices, or at least 15 ophthalmic devices. In certain embodiments, the plurality of ophthalmic devices introduced into the instilled eye is in the range of between 3-20, between 4-20, between 5-20, between 6-20, between 3-10, between 10-20, or between 5-15 ophthalmic devices. In certain embodiments, the plurality of ophthalmic devices introduced into the instilled eye is 3, 4, 5, 6, 7, 8, 9, 10, 11, 12, 13, 14, 15, 16, 17, 18, 19, or 20 ophthalmic devices. In specific embodiments, the plurality of ophthalmic devices positioned around the circumference of the drainage angle are placed in, proximate, or adjacent to at least 2 or at least 3 of the ocular drainage structures identified in the instilled patient's eye. In specific embodiments, the ophthalmic device is a stent. In specific embodiments, at least 1 or at least 2 stents of the plurality of stents are placed in, proximate, or adjacent to, 1 or 2 identified ocular drainage structures located approximately opposite a main collector channel. In specific embodiments, the identified ocular drainage structure is a collector channel. In specific embodiments, the identified ocular drainage structure is the canal of Schlemm. In specific embodiments, the identified ocular drainage structure is the trabecular meshwork. In specific embodiments, the identified ocular drainage structure is the suprachoroidal space. In specific embodiments, the identified ocular drainage structure is the subconjunctival space. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the plurality of ophthalmic devices introduced into the instilled eye, according to the method disclosed herein, are positioned around the circumference of the drainage angle in such a manner as to maximize the angle between at least two of the plurality of ophthalmic devices around the circumference of said drainage angle. For example, in certain embodiments, the plurality of ophthalmic devices introduced into the instilled eye are positioned around the circumference of the drainage angle such that at least 2 of the plurality of ophthalmic devices are separated from each other by 10-30 degrees, 10-45 degrees, 30-45 degrees, 60-120 degrees, 90-270 degrees, 120-240 degrees, 150-210 degrees, 150-180 degrees, or 170-190 degrees, around the circumference of said drainage angle. For example, in certain embodiments, the plurality of ophthalmic devices introduced into the instilled eye are positioned around the circumference of the drainage angle such that at least 3 of the plurality of ophthalmic devices are separated from each other by 10-30 degrees, 30-120 degrees, 30-60 degrees, 40-100 degrees, 50-90 degrees, 60-120 degrees, 80-120 degrees, 90-120 degrees, or 100-120 degrees, around the circumference of said drainage angle. For example, in certain embodiments, the plurality of ophthalmic devices introduced into the instilled eye are positioned around the circumference of the drainage angle such that at least 4 of the plurality of ophthalmic devices are separated from each other by 10-30 degrees, 30-50 degrees, 30-80 degrees, 30-90 degrees, 50-70 degrees, or 60-90 degrees, around the circumference of said drainage angle.

In certain embodiments, according to the methods provided herein, the plurality of ophthalmic devices are positioned in, near or adjacent to the ocular drainage structure identified in the instilled patient's eye around the circumference of the drainage angle.

In certain embodiments, according to the methods provided herein, the plurality of ophthalmic devices are positioned in, near or adjacent to each of the ocular drainage structures identified in the instilled patient's eye around the circumference of the drainage angle.

In certain embodiments, according to the methods provided herein, the plurality of ophthalmic devices are positioned in, near or adjacent to a plurality of the ocular drainage structures identified in the instilled patient's eye around the circumference of the drainage angle.

In certain embodiments, according to the methods provided herein, the plurality of ophthalmic devices are positioned in, near or adjacent to an ocular drainage structure identified in the instilled patient's eye around the circumference of the drainage angle.

In certain embodiments, according to the methods provided herein, the plurality of ophthalmic devices are positioned in, near or adjacent to 2 ocular drainage structures identified in the instilled patient's eye around the circumference of the drainage angle.

In certain embodiments, according to the methods provided herein, the plurality of ophthalmic devices are positioned in, near or adjacent to 3 ocular drainage structures identified in the instilled patient's eye around the circumference of the drainage angle.

In certain embodiments, according to the methods provided herein, the plurality of ophthalmic devices are positioned in, near or adjacent to 4 ocular drainage structures identified in the instilled patient's eye around the circumference of the drainage angle.

In certain embodiments, according to the methods provided herein, the plurality of ophthalmic devices are positioned in, near or adjacent to 5 ocular drainage structures identified in the instilled patient's eye around the circumference of the drainage angle.

In certain embodiments, according to the methods provided herein, the plurality of ophthalmic devices are positioned in, near or adjacent to between 2-20 ocular drainage structures, 3-20 ocular drainage structures, 4-20 ocular drainage structures, 5-20 ocular drainage structures, 2-10 ocular drainage structures, 3-10 ocular drainage structures, 4-10 ocular drainage structures, 5-10 ocular drainage structures, 2-5 ocular drainage structures, or 3-5 ocular drainage structures, identified in the instilled patient's eye around the circumference of the drainage angle.

In certain embodiments, according to the methods provided herein, the plurality of ophthalmic devices are positioned in, near or adjacent to 2, 3, 4, 5, 6, 7, 8, 9, 10, 11, 12, 13, 14, 15, 16, 17, 18, 19, or 20 ocular drainage structures, identified in the instilled patient's eye around the circumference of the drainage angle.

In certain embodiments, according to the methods provided herein, the plurality of ophthalmic devices are positioned in, near or adjacent to 2, 3, 4, 5, 6, 7, 8, 9, or 10 ocular drainage structures, identified in the instilled patient's eye around the circumference of the drainage angle. In certain embodiments, according to the methods provided herein, the plurality of ophthalmic devices are positioned in, near or adjacent to 2, 3, 4, 5, or 6 ocular drainage structures, identified in the instilled patient's eye around the circumference of the drainage angle.

In certain embodiments, according to the methods provided herein, the plurality of ophthalmic devices are positioned in, near or adjacent to 3, 4, 5, 6, 7, 8, 9, 10, 11, 12, 13, 14, 15, 16, 17, 18, 19, or 20 ocular drainage structures, identified in the instilled patient's eye around the circumference of the drainage angle.

In certain embodiments, according to the methods provided herein, the plurality of ophthalmic devices are positioned in, near or adjacent to 3, 4, 5, 6, 7, 8, 9, or 10 ocular drainage structures, identified in the instilled patient's eye around the circumference of the drainage angle.

In certain embodiments, according to the methods provided herein, the plurality of ophthalmic devices are positioned in, near or adjacent to 3, 4, 5, 6, 7, 8, or 9 ocular drainage structures, identified in the instilled patient's eye around the circumference of the drainage angle. In certain embodiments, according to the methods provided herein, the plurality of ophthalmic devices are positioned in, near or adjacent to 3, 4, 5, or 6 ocular drainage structures, identified in the instilled patient's eye around the circumference of the drainage angle.

In certain embodiments, according to the methods provided herein, between 1-5, between 2-5, between 2-4, or between 1-3 ophthalmic devices of the plurality of ophthalmic devices are positioned in, near or adjacent to the ocular drainage structures, identified in the instilled patient's eye around the circumference of the drainage angle.

In certain embodiments, according to the methods provided herein, between 2-5 ophthalmic devices of the plurality of ophthalmic devices are positioned in, near or adjacent to the ocular drainage structures, identified in the instilled patient's eye around the circumference of the drainage angle.

In certain embodiments, according to the methods provided herein, placement of the first ophthalmic device into the patient's eye is after instillation with the ophthalmic composition comprising Indigo Carmine, wherein the first ophthalmic device is positioned in, near or adjacent to an Indigo Carmine identified ocular drainage structure in the instilled patient's eye around the circumference of the drainage angle. According to the methods provided herein, the methods provided herein allow for the placement of the ophthalmic device(s) without the need of guessing or estimating the position of an ocular drainage structure.

In certain embodiments, according to the methods provided herein, the ocular drainage structure identified in the instilled patient's eye around the circumference of the drainage angle is selected from the group consisting of: a fine vessel, an aqueous vein, an episcleral vein, an interscleral channel (e.g., collector channel), a collector channel, a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

In certain embodiments, according to the methods provided herein, the ocular drainage structure identified in the instilled patient's eye around the circumference of the drainage angle is selected from the group consisting of: an aqueous vein, an episcleral vein, an interscleral channel (e.g., collector channel), a collector channel, a collector channel/aqueous/episcleral vein system, an aqueous drainage system, a conjunctival venous system, a subconjunctival space, a trabecular meshwork, a canal of Schlemm, a suprachoroidal space, or a scleral spur.

In certain embodiments, according to the methods provided herein, the ocular drainage structure identified in the instilled patient's eye around the circumference of the drainage angle is selected from the group consisting of: an interscleral channel (e.g., collector channel), a collector channel, a collector channel/aqueous/episcleral vein system, an aqueous drainage system, a conjunctival venous system, a subconjunctival space, a trabecular meshwork, a canal of Schlemm, a suprachoroidal space, a scleral spur.

In certain embodiments, according to the methods provided herein, the ocular drainage structure identified in the instilled patient's eye around the circumference of the drainage angle is an interscleral channel (e.g., collector channel).

In certain embodiments, according to the methods provided herein, the ocular drainage structure identified in the instilled patient's eye around the circumference of the drainage angle a collector channel.

In certain embodiments, according to the methods provided herein, the ocular drainage structure identified in the instilled patient's eye around the circumference of the drainage angle is a canal of Schlemm.

In certain embodiments, according to the methods provided herein, the ocular drainage structure identified in the instilled patient's eye around the circumference of the drainage angle is a conjunctival venous system.

In certain embodiments, according to the methods provided herein, the ocular drainage structure identified in the instilled patient's eye around the circumference of the drainage angle is a subconjunctival space.

In certain embodiments, according to the methods provided herein, the ocular drainage structure identified in the instilled patient's eye around the circumference of the drainage angle is a trabecular meshwork.

In certain embodiments, according to the methods provided herein, the ocular drainage structure identified in the instilled patient's eye around the circumference of the drainage angle is an aqueous drainage system.

In certain embodiments, according to the methods provided herein, the ocular drainage structure identified in the instilled patient's eye around the circumference of the drainage angle is a suprachoroidal space.

In certain embodiments, according to the methods provided herein, the plurality of ophthalmic devices are positioned in, near or adjacent to 2 interscleral channels (e.g., collector channels) identified in the instilled patient's eye around the circumference of the drainage angle.

In certain embodiments, according to the methods provided herein, the plurality of ophthalmic devices are positioned in, near or adjacent to 3 interscleral channels (e.g., collector channels) identified in the instilled patient's eye around the circumference of the drainage angle.

In certain embodiments, according to the methods provided herein, the plurality of ophthalmic devices are positioned in, near or adjacent to 4 interscleral channels (e.g., collector channels) identified in the instilled patient's eye around the circumference of the drainage angle.

In certain embodiments, according to the methods provided herein, the plurality of ophthalmic devices are positioned in, near or adjacent to 5 interscleral channels (e.g., collector channels) identified in the instilled patient's eye around the circumference of the drainage angle.

In certain embodiments, according to the methods provided herein, the plurality of ophthalmic devices are positioned in, near or adjacent to between 2-20 interscleral channels (e.g., collector channels), 3-20 interscleral channels (e.g., collector channels), 4-20 interscleral channels (e.g., collector channels), 5-20 interscleral channels (e.g., collector channels), 2-10 interscleral channels (e.g., collector channels), 3-10 interscleral channels (e.g., collector channels), 4-10 interscleral channels (e.g., collector channels), 5-10 interscleral channels (e.g., collector channels), 2-5 interscleral channels (e.g., collector channels), or 3-5 interscleral channels (e.g., collector channels), identified in the instilled patient's eye around the circumference of the drainage angle. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, the plurality of ophthalmic devices introduced into the instilled eye, according to the method disclosed herein, are positioned around the circumference of the drainage angle to maintain physiological aqueous humor circulation of the patient's eye following the ocular surgery. In certain embodiments, the plurality of ophthalmic devices introduced into the instilled eye are positioned around the circumference of the drainage angle to maintain corneal nutrition of the patient's eye following the ocular surgery. In certain embodiments, the plurality of ophthalmic devices introduced into the instilled eye are positioned around the circumference of the drainage angle to maintain adequate corneal nutrition of the patient's eye following the ocular surgery. In certain embodiments, the plurality of ophthalmic devices introduced into the instilled eye are positioned around the circumference of the drainage angle to maintain uniform corneal nutrition of the patient's eye following the ocular surgery. In certain embodiments, the plurality of ophthalmic devices introduced into the instilled eye are positioned around the circumference of the drainage angle to maintain corneal endothelial cell density of the patient's eye following the ocular surgery. In certain embodiments, the plurality of ophthalmic devices introduced into the instilled eye are positioned around the circumference of the drainage angle to mitigate corneal endothelial cell loss of the patient's eye following the ocular surgery. In certain embodiments, the plurality of ophthalmic devices introduced into the instilled eye are positioned around the circumference of the drainage angle to form a plurality of circulation patterns of the aqueous humor within the patient's eye following the ocular surgery. In certain embodiments, the plurality of ophthalmic devices introduced into the instilled eye are positioned around the circumference of the drainage angle to form a plurality of drain flow patterns of the aqueous humor within the patient's eye following the ocular surgery. In certain embodiments, the plurality of ophthalmic devices introduced into the instilled eye are positioned around the circumference of the drainage angle to maintain an average IOP level of at least 10 mmHg and less than 15 mmHg.

In certain embodiments, the plurality of ophthalmic devices introduced into the instilled eye, according to the method disclosed herein, are positioned around the circumference of the drainage angle; and wherein the ocular surgery is selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique.

In certain embodiments, the plurality of positions or plurality of locations the plurality of ophthalmic devices are introduced into the instilled eye around the circumference of the drainage angle, according to the method disclosed herein, are selected from the group consisting of: a fine vessel, an aqueous vein, an episcleral vein, a collector channel, a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

In certain embodiments, according to the methods provided herein, the ophthalmic device is a stent, for example, a glaucoma stent, a suprachoroidal stent, iStent (Glaukos), Hydrus microstent (Ivantis), CyPass (Alcon), iStent Supra (Glaukos), Solx gold shunt (Solx), XEN Gel Stent (Allergan), and/or InnFocus MicroShunt (InnFocus, Santen). In certain embodiments, the plurality of ophthalmic devices comprises a glaucoma stent and/or a suprachoroidal stent.

In certain embodiments, according to the methods provided herein, at least one of the plurality of ophthalmic devices introduced into the instilled eye that is positioned around the circumference of the drainage angle to form a plurality of drain flow patterns of the aqueous humor within the patient's eye following the ocular surgery is located approximately opposite a main collector channel.

In certain embodiments, according to the methods provided herein, at least one of the plurality of ophthalmic devices introduced into the instilled eye that is positioned around the circumference of the drainage angle to form a plurality of drain flow patterns of the aqueous humor within the patient's eye following the ocular surgery is located approximately opposite a main collector channel identified by the instilled composition.

In certain embodiments, according to the methods provided herein, a plurality or each of the plurality of ophthalmic devices has an internal diameter of between 5-500 microns, 5-450 microns, 5-420 microns, 5-400 microns, 5-375 microns, 5-350 microns, 5-325 microns, 5-300 microns, 5-275 microns, 5-205 microns, 5-250 microns, 5-225 microns, 5-200 microns, 5-175 microns, 5-150 microns, 5-125 microns, 5-100 microns, or 5-50 microns, 50-500 microns, 50-400 microns, 50-300 microns, 50-200 microns, 50-100 microns, 100-500 microns, 100-400 microns, 100-300 microns, 100-200 microns, 200-500 microns, 200-400 microns, 200-300 microns, 300-500 microns, 300-400 microns, or 400-500 microns, plus or minus 4 microns. In specific embodiments, the plurality of ophthalmic devices is 2-10 ophthalmic devices, wherein each ophthalmic device independently has an internal diameter of about 50-80 microns, plus or minus 4 microns. In specific embodiments, the plurality of ophthalmic devices is 3-10 ophthalmic devices, wherein each ophthalmic device independently has an internal diameter of about 50-80 microns, plus or minus 4 microns. In specific embodiments, the plurality of ophthalmic devices is 2-6 ophthalmic devices, wherein each ophthalmic device independently has an internal diameter of about 50-80 microns, plus or minus 4 microns. In specific embodiments, the plurality of ophthalmic devices is 3-6 ophthalmic devices, wherein each ophthalmic device independently has an internal diameter of about 50-80 microns, plus or minus 4 microns. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, according to the methods provided herein, a plurality or each of the plurality of ophthalmic devices has an internal diameter of about 5, 10, 15, 20, 30, 40, 50, 60, 70, 80, 90, 100, 125, 150, 175, 200, 225, 250, 275, 300, 325, 350, 375, 400, 425, 450, 475, or 500 microns, plus or minus 4 microns. In certain embodiments, the placement of the ophthalmic device near, proximate, or adjacent to, the identified ocular structure is within a distance of 1-5 inner diameters of said ophthalmic device, such as within 1-3 inner diameters or within 1-2 inner diameters of said ophthalmic device. In certain embodiments, the distance of placing the ophthalmic device near, proximate, or adjacent to, the identified ocular structure is within 5, 4, 3, 2, or 1 inner diameters of said ophthalmic device. In certain embodiments, the distance of placing the ophthalmic device near, proximate, or adjacent to, the identified ocular structure is within 2 inner diameters of said ophthalmic device. In certain embodiments, the distance of placing the ophthalmic device near, proximate, or adjacent to, the identified ocular structure is within 1 inner diameter of said ophthalmic device.

In certain embodiments, according to the methods provided herein, a plurality or each of the plurality of ophthalmic devices has a length of between 5-20 mm, 5-19 mm, 5-18 mm, 5-17 mm, 5-16 mm, 5-15 mm, 5-10 mm, 10-20 mm, 10-19 mm, 10-18 mm, 10-17 mm, 10-16 mm, or 10-15 mm, plus or minus 0.5 mm.

In certain embodiments, according to the methods provided herein, a plurality or each of the plurality of ophthalmic devices has a length of about 5, 6, 7, 8, 9, 10, 11, 12, 13, 14, 15, 16, 17, 18, 19, or 20 mm, plus or minus 0.5 mm.

In certain embodiments, according to the methods provided herein, a plurality or each of the plurality of ophthalmic devices is composed of a material, or coated with a material, or contains an inner layer of a material, selected from the group consisting of: poly(styrene-block-isobutylene-block-styrene (SIBS), Polyimide, heparin-coated non-ferromagnetic titanium, nitinol, collagen-derived porcine gelatine cross-linked with glutaraldehyde, or derivatives thereof.

In certain embodiments, according to the methods provided herein, a plurality or each of the plurality of ophthalmic devices is composed of a material, or coated with a material, or contains an inner layer of a material, of poly(styrene-block-isobutylene-block-styrene (SIBS), or derivative thereof.

In certain embodiments, according to the methods provided herein, internal diameters of the plurality of ophthalmic devices have sufficient flow resistance such that the resulting IOP of the treated patient's eye maintains a level of at least 10 mm Hg.

In certain embodiments, according to the methods provided herein, each of the plurality of ophthalmic devices has an internal diameter with sufficient flow resistance such that the resulting IOP of the treated patient's eye maintains a level of at least 10 mm Hg.

In certain embodiments, according to the methods provided herein, each of the plurality of ophthalmic devices has an internal diameter with sufficient flow resistance such that the resulting IOP of the treated patient's eye maintains a level of at least 10 mm Hg and no more than 15 mm Hg.

In certain embodiments, according to the methods provided herein, the patient's IOP levels following introduction of the plurality of ophthalmic devices maintains an average IOP level of at least 10 mm Hg and below 21 mm Hg.

In certain embodiments, according to the methods provided herein, the patient's average IOP levels following introduction of the plurality of ophthalmic devices are at least 10 mm Hg and no more than 15 mm Hg.

In certain embodiments, according to the methods provided herein, the patient's average IOP levels following introduction of the plurality of ophthalmic devices are at least 10 mm Hg and no more than 14 mm Hg.

In certain embodiments, according to the methods provided herein, the patient's average IOP levels following introduction of the plurality of ophthalmic devices are at least 10 mm Hg and no more than 13 mm Hg.

In certain embodiments, according to the methods provided herein, the patient's IOP levels following introduction of the plurality of ophthalmic devices maintains and/or achieves an average IOP level, relative to a human of similar age having a healthy average IOP level.

In certain embodiments, according to the methods provided herein, the patient's density of corneal endothelium cells following introduction of the plurality of ophthalmic devices maintains and/or achieves an average of between 3000-4000 cells per square mm.

In certain embodiments, according to the methods provided herein, the patient's density of corneal endothelium cells located centrally of the cornea following introduction of the plurality of ophthalmic devices maintains and/or achieves an average of between 3000-4000 cells per square mm.

In certain embodiments, according to the methods provided herein, the patient's density of corneal endothelium cells located on the periphery of the cornea following introduction of the plurality of ophthalmic devices maintains and/or achieves an average of between 3000-4000 cells per square mm.

In certain embodiments, according to the methods provided herein, the patient's density of corneal endothelium cells located centrally and located on the periphery of the cornea following introduction of the plurality of ophthalmic devices maintains and/or achieves an average of between 3000-4000 cells per square mm.

In certain embodiments, according to the methods provided herein, the patient's density of corneal endothelium cells following introduction of the plurality of ophthalmic devices maintains and/or achieves a density level, relative to a human of similar age having a healthy corneal endothelium layer.

In certain embodiments, according to the methods provided herein, the aqueous humor circulation of the patient's eye following introduction of the plurality of ophthalmic devices maintains and/or achieves a level of a healthy human of similar age having a healthy aqueous humor circulation, such as determined by fluorophtomtetry.

In certain embodiments, according to the methods provided herein, the cell circulation in the anterior chamber of the patient's eye following introduction of the plurality of ophthalmic devices maintains and/or achieves a level of a healthy human of similar age having a healthy cell circulation in the anterior chamber, such as determined by optical coherence.

In certain embodiments, according to the methods provided herein, the thickness of the corneal endothelium of the patient's eye following introduction of the plurality of ophthalmic devices maintains and/or achieves a level of thickness in a human of similar age having a healthy corneal endothelium layer.

In certain embodiments, according to the methods provided herein, the health of the corneal endothelium of the patient's eye following introduction of the plurality of ophthalmic devices maintains and/or achieves a level of a healthy human of similar age having a healthy corneal endothelium layer.

In certain embodiments, according to the methods provided herein, the health of the aqueous humor circulation of the patient's eye following introduction of the plurality of ophthalmic devices maintains and/or achieves a level of a healthy human of similar age having a healthy aqueous humor circulation.

In certain embodiments, according to the methods provided herein, the ophthalmic device is a stent.

In certain embodiments, according to the methods provided herein, the plurality of ophthalmic devices is a plurality of stents.

In a further embodiment, the ophthalmic composition, or the method, of any one of the above embodiments and any one or more of the further embodiments herein, wherein each of the plurality of ophthalmic devices is a stent.

In certain embodiments, according to the methods provided herein, the ophthalmic device is a glaucoma stent or suprachoroidal stent.

In certain embodiments, according to the methods provided herein, the plurality of ophthalmic devices comprises: a glaucoma stent, a suprachoroidal stent, iStent (Glaukos), Hydrus microstent (Ivantis), CyPass (Alcon), iStent Supra (Glaukos), Solx gold shunt (Solx), XEN Gel Stent (Allergan), and/or InnFocus MicroShunt (InnFocus, Santen).

In certain embodiments, according to the methods provided herein, the plurality of ophthalmic devices comprises a glaucoma stent and/or a suprachoroidal stent.

### EXAMPLES

The following ophthalmic compositions (dye solutions) used were aqueous solutions containing Indigo Carmine+/−Trypan Blue, wherein the concentration of the Trypan Blue present is 0.1 wt. % or less.

Dye Instillation Procedure:

After an incision is made into the anterior chamber of the eye, a small amount of aqueous humor is expressed and the dye solution is instilled by injection into the anterior chamber. The dye solution is typically left in place for 30 seconds, during which time it stains the anterior capsule of the lens. Excess dye solution is then expelled, usually using a viscoelastic device or flushed out with a balanced salt solution.

Ocular Surgery:

The dye instillation procedure detailed above is suitable for ocular surgeries, such as cataract surgery, glaucoma surgery, combination cataract/glaucoma surgery, minimally invasive glaucoma surgery (MIGS), retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, ocular surface diagnostic technique, and combinations thereof.

For cataract surgery, this dye instillation procedure enables enhanced visualization of the lens capsule when it is incised.

For glaucoma surgery, such as minimally invasive glaucoma surgery (MIGS), after instillation of the dye solution, stents may be placed in the now stained eye:

i) if a stent is to be placed in canal of Schlemm, the site of egress of the dye solution in the episcleral veins will be noted and stents (like iStent) are to be placed adjacent to these areas of maximal outflow.

ii) if the stent is to be placed in the suprachoroidal space (CyPass), the dye solution (containing Indigo Carmine+/−Trypan Blue) stains both canal of Schelmm and trabecular meshwork, thereby enabling precise identification of structures in the angle of the eye, so the stent can be more precisely and accurately placed.

After insertion of the stent(s), more dye solution can be flushed through the anterior chamber, via the stents and out of the anterior chamber, to confirm the potency of the placed stent(s).

For Ab Externo Canaloplasty (ABeC), after instillation of the dye solution, the stained collector channel(s) and episcleral vein(s) are identified, so one or more of the collector channels and episcleral veins can be cannulated. Some flushing with balanced salt will then be carried out to clear some of the dye solution. Next, a dye solution containing Indigo Carmine+/− Trypan Blue, either in the usual balanced salt solution or in a viscolesatic device, will be injected retrogradely to fill the canal of Schlemm. In some instances, the concentration of the Indigo Carmine in the dye injected into the canal of Schlemm may be more concentrated than used initially so as to better visualize the canal, and in some instances, multiple injections will be required into each of the major episcleral veins.

For corneal procedures, such as Descemet's Membrane Endothelial Keratoplasty (DMEK), the tissue to be transplanted is prepared and it is placed in the dye solution long enough for staining to occur, though the time is kept to a minimum to reduce any possibility of toxicity.

### Example 1

Dye solutions containing 0.004 wt. %, 0.04 wt. %, and 0.4 wt. % of Indigo Carmine (prepared by serial dilutions of a 0.4 wt. % Indigo Carmine solution provided by Micro-Tech (Nanjing) Co.) were instilled by injection into the anterior chamber of seven eye bank eyes following the dye solution instillation procedure detailed above.

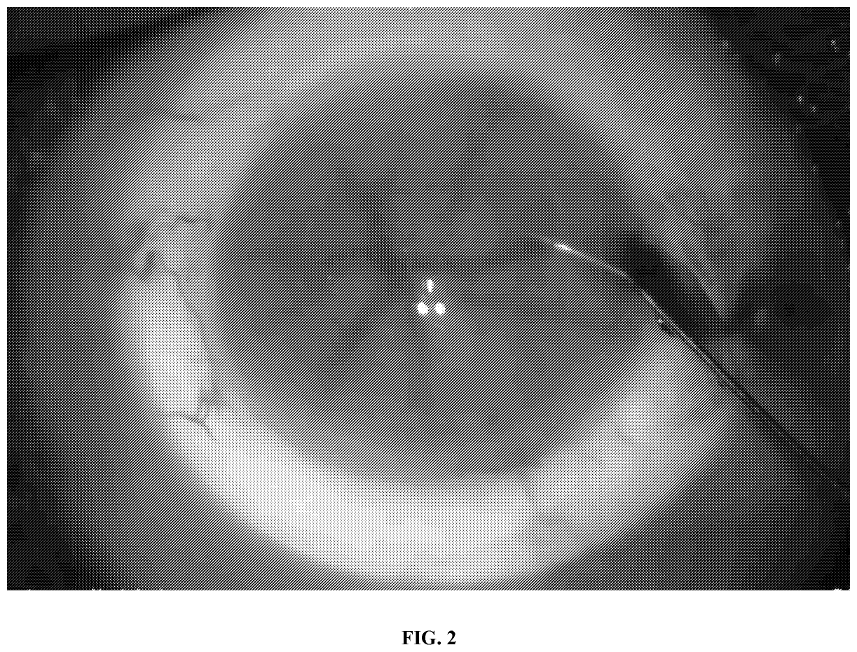

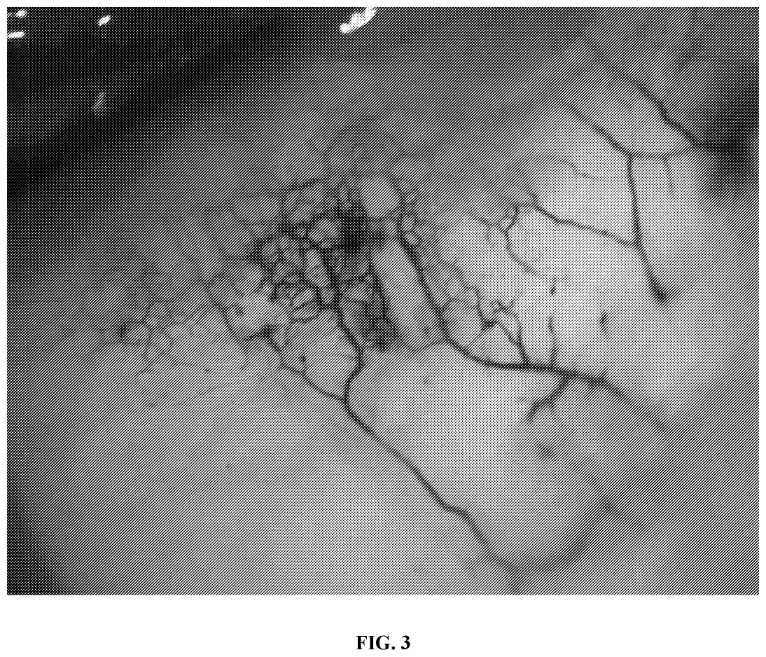

After a short delay from the start of the infusion/injection, the Indigo Carmine dye appeared in the fine vessels in the conjunctiva adjacent to the limbus, the aqueous veins and the conjunctival venous system. The Indigo Carmine dye was easily visible to the naked eye of the surgeon as it appeared on the surface of the dye instilled eye, and detailed branching of the vessels delineated were visible using a standard operating microscope routinely used in glaucoma surgery (see FIG. 2). Special imaging techniques were not required. The Indigo Carmine dye was also observed in the deep scleral plexus, once the conjunctiva was reflected away (see FIG. 3). The Indigo Carmine dye, at each concentration noted above, was visible to the naked eye of the surgeon as it appeared in the collector channels on the surface of the eye after it had passed through the drainage system.

The observed staining achieved by the Indigo Carmine dye solution is in contrast with that observed with other ophthalmic dyes, such as Trypan Blue (e.g., VISIONBLUE®, containing 0.06 wt. % Trypan Blue), Brilliant Blue, Patent Blue, and Indocyanine Green. Specifically, unlike with the Indigo Carmine dye solution, after injection of the other ophthalmic dyes (Trypan Blue, Brilliant Blue, Patent Blue, or Indocyanine Green), the dye appears to remain within the eye. This is likely due to the fact that these other ophthalmic dyes are bound within the angle to entities such as glycosaminoglycans (GAGS), proteins or to other molecules or structures within the drainage angle. Similarly, an injection with the ophthalmic dye Fluorescein tends to diffuse through the tissues and is not very useful in specifically detecting the drainage pathways.

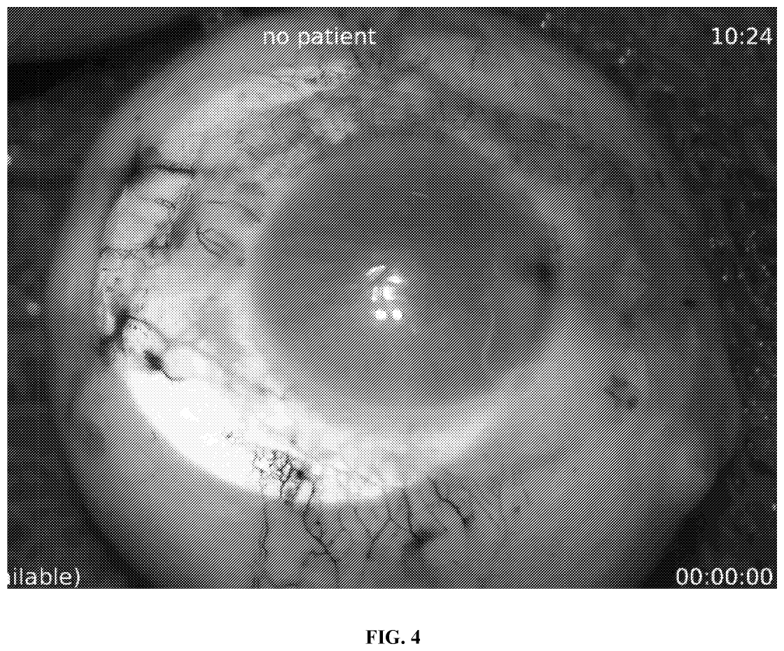

As predicted from recent studies on the collector channels (see, e.g., Hann, C. R., et al., “Anatomic changes in Schlemm's canal and collector channels in normal and primary open-angle glaucoma eyes using low and high perfusion pressures”, Invest. Ophthalmol. Vis. Sci., 2014 Aug. 19, 55(9):5834-41; Wirbelauer, C., et al., “Role of Intraoperative Indirect Channelography in Glaucoma Stent Implantation”, Klin. Monbl. Augenheilkd., 2017, 234:1378-1386; Huang, A. S., et al., “Aqueous Angiography: Aqueous Humor Outflow Imaging in Live Human Subjects”, Ophthalmology, 2017, 124:1249-1251; and Saraswathy, S., et al., “Aqueous Angiography: Real-Time and Physiologic Aqueous Humor Outflow Imaging”, PLoS One, 2016 Jan. 25, 11(1):e0147176), the distribution of this drainage pattern is not uniform, which may be an age related phenomenon (see FIG. 4, and see FIG. 2).

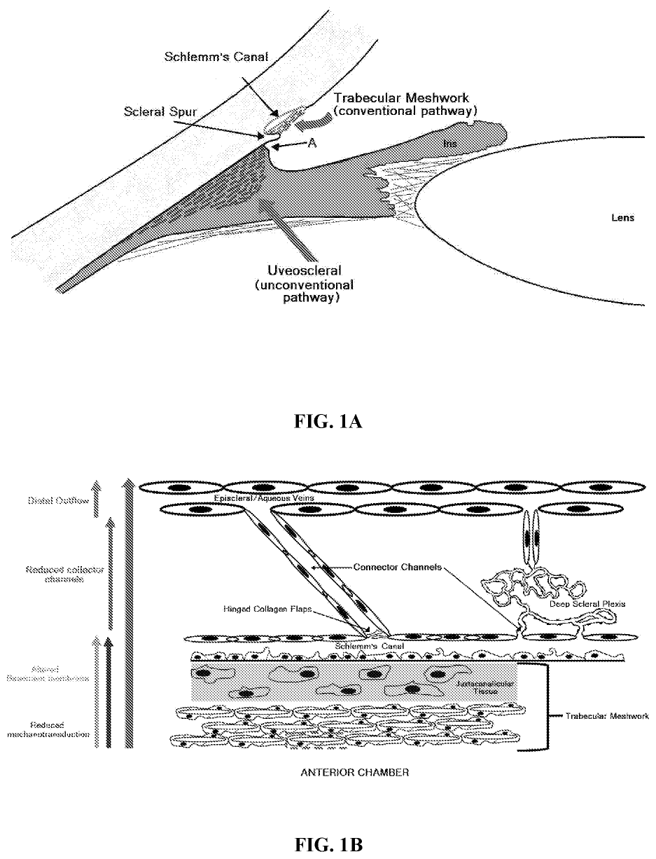

As can be observed in FIG. 5, the Indigo Carmine dye stains the trabecular meshwork lightly and enters the canal of Schlemm, which stains heavily, allowing for easier identification of these structures. The staining of these structures is critical for the insertion of modern glaucoma stents, as these landmarks can be difficult to see intraoperatively. FIG. 5 shows how the trabecular meshwork and the canal of Schlemm are stained, bringing them into stark contrast against the surrounding tissue. This allows accurate placement of stents either in the canal of Schlemm or into the suprachoroidal space—since this technique require identification of the scleral spur (FIG. 1A). The posterior aspect of the trabecular meshwork attaches to the scleral spur, which can now be accurately visualized by the Indigo Carmine dye (FIG. 5). Suprachoroidal stents are inserted just posterior to the scleral spur, and into the suprachoridal space.

The trabecular meshwork is particularly well seen with a dye solution containing a combination of Indigo Carmine and Trypan Blue, since the Trypan Blue particularly enhances trabecular meshwork staining.

Utilization of the Indigo Carmine dye for ophthalmic staining facilitates the determination of both the location and the type of stent to be used. In situations where there is little outflow into the collector channel/aqueous/episcleral vein system, then a suprachoroidal stent would be used, since there is “nowhere to go” via the conventional drainage pathway in the angle of the eye. In situations where collector channels are identified, then a stent placed in the canal of Schlemm may be preferable and these stents would be placed adjacent to the sectors with good collector channel drainage. In certain situations, several stents could be placed in this way to take advantage of good “down-stream” drainage.

### Example 2

To investigate aqueous humor circulation and flow, the anterior chamber geometry was generated in solid modelling software package (Fusion 3D). This computer model was utilized to investigate what impact stent placement, including what impact the number of stents placed, has on various ocular properties, such as aqueous humor circulation, velocity of flow, and ocular pressure. This computer model was

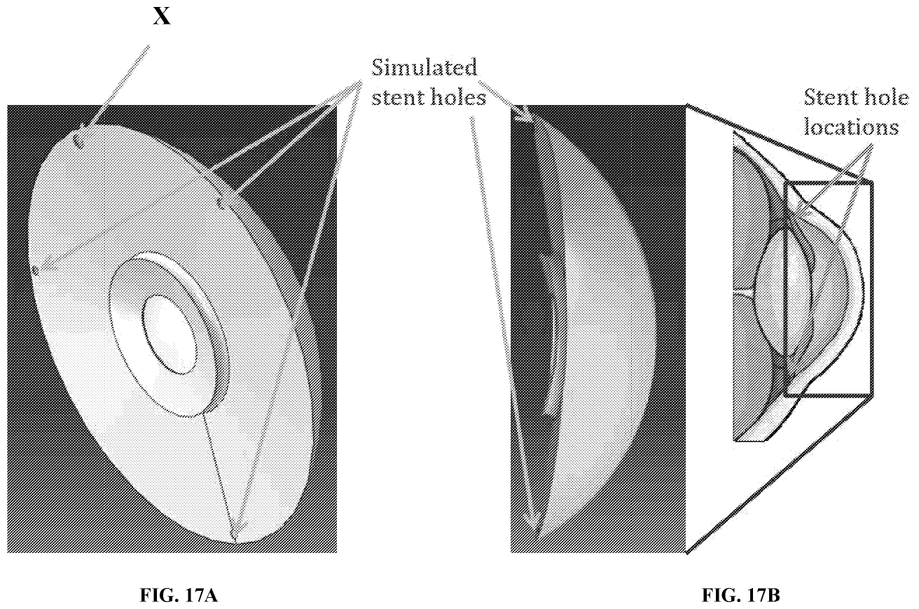

In the model utilized, the stent hole(s) (simulated as outlets in the computer model) were located and placed at 120 degrees apart (see FIG. 17A) in the anterior chamber (see FIG. 17B), and each stent hole (outlet) had a diameter of 0.3 mm. As shown in FIG. 17A, one additional hole was included as a modelling requirement (labeled “X”), but this hole had no fluid interaction and no fluid passed through it. The aqueous humor fluid properties utilized in the computer model were taken from the literature (see Dvoriashyna, M., et al., Ocular Fluid Dynamics 2019). The generated model was analyzed using ABAQUS Computational Fluid Dynamics (CFD) software.

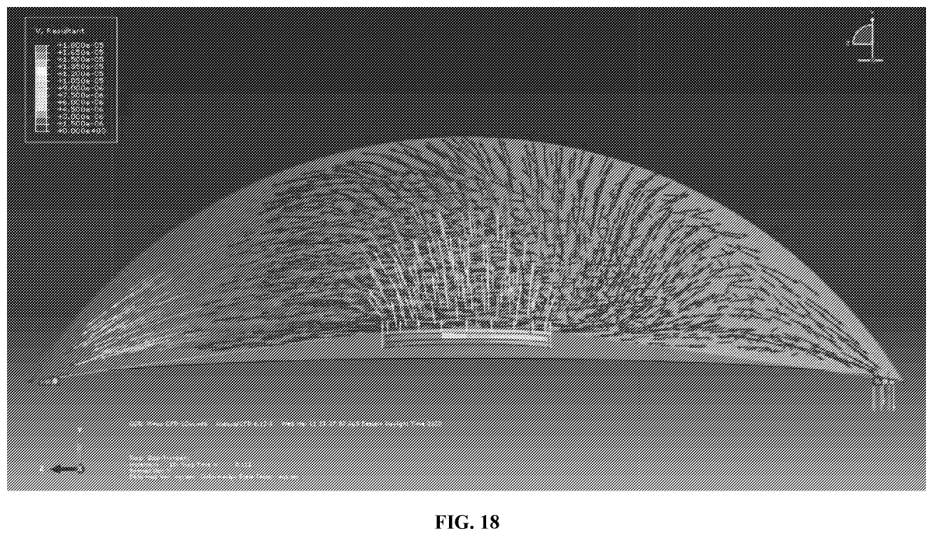

Using the generated model, the velocity vectors simulating the flow of the aqueous humour within the anterior chamber as a result of including 1 outlet is shown in FIG. 18. Computer generated models showing the results of including 2, 3, or 4 outlets, were also conducted (figures not shown). The data and results obtained from these computer generated models verified the results observed in the in vitro model described below.

An in vitro model of an anterior chamber filled with an aqueous solution containing glitter was used to be able to track aqueous flow within the modeled anterior chamber so as to investigate the impact on aqueous flow as a result of inserting one or more stents, such as 1, 2, 3, or 4 stents, into locations along the perimeter of the anterior chamber, similar to the positions illustrated in FIGS. 17A-17B (a video showing these results was taken and is described below, but the video is not shown here). To demonstrate (and track) the impact on aqueous flow as a result of inserting one or more stents, the glitter-containing aqueous solution was continuously infused into the in vitro model from the center posterior portion of the modeled anterior chamber and the infusion was in posterior to anterior direction with the anterior chamber. Insertion of a single stent at a position along the perimeter of the in vitro model of the anterior chamber resulted in a non-uniform and unevenly distributed flow of the infused glitter-containing aqueous solution throughout the modeled anterior chamber. The uniformity and distribution of the solution improved slightly with the insertion of a second stent positioned 120 degrees apart from the first stent about the perimeter of the in vitro model of the anterior chamber, but the resulting circulation and flow of the solution was still not uniform or evenly distributed throughout the modeled chamber. Insertion of a third stent, positioned 120 degrees apart from the first and second stents about the perimeter of the in vitro model of the anterior chamber, resulted in a more uniform and evenly distributed circulation of the solution within the modeled anterior chamber. With three stents, the number of locations showing minimal flow relative to the rest of the chamber (as observed with a single or two stents) was greatly decreased.

### EXEMPLARY EMBODIMENTS

A1. In an embodiment, an ophthalmic composition, comprising Indigo Carmine.

A2. In an embodiment, an ophthalmic composition, comprising Indigo Carmine and Trypan Blue.

A3. In an embodiment, a method for ocular surgery in a patient in need thereof, comprising instilling an ophthalmic composition comprising Indigo Carmine into the patient's eye.

A4. In an embodiment, a method for ocular surgery in a patient in need thereof, comprising instilling an ophthalmic composition comprising Indigo Carmine and Trypan Blue into the patient's eye.

A5. In an embodiment, a method for ocular surgery in a patient in need thereof, comprising: instilling an ophthalmic composition comprising Indigo Carmine and an ophthalmic composition comprising Trypan Blue into the patient's eye.

A6. In an embodiment, a method for identifying an intraocular structure(s) or membrane(s) within an eye of a patient in need thereof, comprising instilling an ophthalmic composition comprising Indigo Carmine into the patient's eye.

A7. In an embodiment, a method for identifying an intraocular structure(s) or membrane(s) within an eye of a patient in need thereof, comprising instilling an ophthalmic composition comprising Indigo Carmine and Trypan Blue into the patient's eye.

A8. In an embodiment, a method for identifying an intraocular structure(s) or membrane(s) within an eye of a patient in need thereof, comprising: instilling an ophthalmic composition comprising Indigo Carmine and an ophthalmic composition comprising Trypan Blue into the patient's eye.

A9. In an embodiment, a method for introducing an ophthalmic device into an eye of a patient in need thereof, comprising:
i) instilling an ophthalmic composition comprising Indigo Carmine into the patient's eye; and
ii) introducing the ophthalmic device into the instilled eye.

A10. In an embodiment, a method for introducing an ophthalmic device into an eye of a patient in need thereof, comprising:
i) instilling an ophthalmic composition comprising Indigo Carmine and Trypan Blue into the patient's eye; and
ii) introducing the ophthalmic device into the instilled eye.

A11. In an embodiment, a method for introducing an ophthalmic device into an eye of a patient in need thereof, comprising:
i) instilling an ophthalmic composition comprising Indigo Carmine and an ophthalmic composition comprising Trypan Blue into the patient's eye; and
ii) introducing the ophthalmic device into the instilled eye.

A12. In an embodiment, a method for introducing an ophthalmic device into an eye of a patient in need thereof, comprising:
i) instilling an ophthalmic composition comprising Indigo Carmine into the patient's eye; and
ii) introducing a plurality of ophthalmic devices into the instilled eye, wherein the plurality of ophthalmic devices are positioned around the circumference of the drainage angle to maintain physiological aqueous humor circulation, maintain corneal nutrition, maintain corneal endothelial cell density and/or mitigate corneal endothelial cell loss.

A13. In an embodiment, a method for introducing an ophthalmic device into an eye of a patient in need thereof, comprising:
i) instilling an ophthalmic composition comprising Indigo Carmine and Trypan Blue into the patient's eye; and
ii) introducing a plurality of ophthalmic devices into the instilled eye, wherein the plurality of ophthalmic devices are positioned around the circumference of the drainage angle to maintain physiological aqueous humor circulation, maintain corneal nutrition, maintain corneal endothelial cell density and/or mitigate corneal endothelial cell loss.

A14. In an embodiment, a method for introducing an ophthalmic device into an eye of a patient in need thereof, comprising:
i) instilling an ophthalmic composition comprising Indigo Carmine and an ophthalmic composition comprising Trypan Blue into the patient's eye; and
ii) introducing a plurality of ophthalmic devices into the instilled eye, wherein the plurality of ophthalmic devices are positioned around the circumference of the drainage angle to maintain physiological aqueous humor circulation, maintain corneal nutrition, maintain corneal endothelial cell density and/or mitigate corneal endothelial cell loss.

A15. In an embodiment, a method for introducing an ophthalmic device into an eye of a patient in need thereof, comprising:
i) instilling an ophthalmic composition comprising Indigo Carmine into the patient's eye; and
ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage.

A16. In an embodiment, a method for introducing an ophthalmic device into an eye of a patient in need thereof, comprising:
i) instilling an ophthalmic composition comprising Indigo Carmine and Trypan Blue into the patient's eye; and
ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage.

A17. In an embodiment, a method for introducing an ophthalmic device into an eye of a patient in need thereof, comprising:
i) instilling an ophthalmic composition comprising Indigo Carmine and an ophthalmic composition comprising Trypan Blue into the patient's eye; and
ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage.

A18. In an embodiment, a method for glaucoma surgery of an eye of a patient in need thereof, comprising:
i) instilling an ophthalmic composition comprising Indigo Carmine into the patient's eye; and
ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage.

A19. In an embodiment, a method for glaucoma surgery of an eye of a patient in need thereof, comprising:
i) instilling an ophthalmic composition comprising Indigo Carmine and Trypan Blue into the patient's eye; and
ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage.

A20. In an embodiment, a method for glaucoma surgery of an eye of a patient in need thereof, comprising:
i) instilling an ophthalmic composition comprising Indigo Carmine and an ophthalmic composition comprising Trypan Blue into the patient's eye; and
ii) introducing a plurality of ophthalmic devices into the instilled eye, wherein the plurality of ophthalmic devices are positioned around the circumference of the drainage angle in, near, or adjacent to at least 2, at least 3, at least 4, at least 5, at least 6, at least 7, at least 8, at least 9, at least 10, or more, ocular drainage structures identified in the instilled patient's eye positioned around the circumference of the drainage.

A21. In an embodiment, a method for maintaining physiological aqueous humor circulation of an eye of a patient following ocular surgery of the patient's eye, comprising:
i) instilling an ophthalmic composition comprising Indigo Carmine into the patient's eye;
ii) performing the ocular surgery (e.g., cataract surgery) of the patient's instilled eye;
iii) optionally, further instilling the ophthalmic composition comprising Indigo Carmine into the patient's eye following the ocular surgery; and
iv) introducing a plurality of ophthalmic devices into the instilled eye, wherein the plurality of ophthalmic devices are positioned around the circumference of the drainage angle.

A22. In an embodiment, a method for maintaining physiological aqueous humor circulation of an eye of a patient following ocular surgery of the patient's eye, comprising:
i) instilling an ophthalmic composition comprising Indigo Carmine and Trypan Blue into the patient's eye;
ii) performing the ocular surgery (e.g., cataract surgery) of the patient's instilled eye;
iii) optionally, further instilling the ophthalmic composition comprising Indigo Carmine into the patient's eye following the ocular surgery; and
iv) introducing a plurality of ophthalmic devices into the instilled eye, wherein the plurality of ophthalmic devices are positioned around the circumference of the drainage angle.

A23. In an embodiment, a method for maintaining physiological aqueous humor circulation of an eye of a patient following ocular surgery of the patient's eye, comprising:
i) instilling an ophthalmic composition comprising Indigo Carmine and an ophthalmic composition comprising Trypan Blue into the patient's eye;
ii) performing the ocular surgery (e.g., cataract surgery) of the patient's instilled eye;
iii) optionally, further instilling the ophthalmic composition comprising Indigo Carmine into the patient's eye following the ocular surgery; and
iv) introducing a plurality of ophthalmic devices into the instilled eye, wherein the plurality of ophthalmic devices are positioned around the circumference of the drainage angle.

A24. In an embodiment, a method of ocular surgery of an eye of a patient in need thereof, comprising:
i) instilling an ophthalmic composition comprising Indigo Carmine into the patient's eye;
ii) performing the ocular surgery (e.g., cataract surgery) of the patient's instilled eye;
iii) optionally, further instilling the ophthalmic composition comprising Indigo Carmine into the patient's eye following the ocular surgery; and
iv) introducing a plurality of ophthalmic devices into the instilled eye, wherein the plurality of ophthalmic devices are positioned in a plurality of directions around the circumference of the drainage angle.

A25. In an embodiment, a method of ocular surgery of an eye of a patient in need thereof, comprising:
i) instilling an ophthalmic composition comprising Indigo Carmine and Trypan Blue into the patient's eye;
ii) performing the ocular surgery (e.g., cataract surgery) of the patient's instilled eye;
iii) optionally, further instilling the ophthalmic composition comprising Indigo Carmine into the patient's eye following the ocular surgery; and
iv) introducing a plurality of ophthalmic devices into the instilled eye, wherein the plurality of ophthalmic devices are positioned in a plurality of directions around the circumference of the drainage angle.

A26. In an embodiment, a method of ocular surgery of an eye of a patient in need thereof, comprising:
i) instilling an ophthalmic composition comprising Indigo Carmine and an ophthalmic composition comprising Trypan Blue into the patient's eye;
ii) performing the ocular surgery (e.g., cataract surgery) of the patient's instilled eye;
iii) optionally, further instilling the ophthalmic composition comprising Indigo Carmine into the patient's eye following the ocular surgery; and
iv) introducing a plurality of ophthalmic devices into the instilled eye, wherein the plurality of ophthalmic devices are positioned in a plurality of directions around the circumference of the drainage angle.

A27. In an embodiment, a method for identification of canal of Schlemm within an eye of a patient in need thereof, comprising instilling an ophthalmic composition comprising Indigo Carmine the patient's eye.

A28. In an embodiment, a method for identification of canal of Schlemm within an eye of a patient in need thereof, comprising instilling an ophthalmic composition comprising Indigo Carmine and Trypan Blue the patient's eye.

A29. In an embodiment, a method for identification of canal of Schlemm within an eye of a patient in need thereof, comprising instilling an ophthalmic composition comprising Indigo Carmine and an ophthalmic composition comprising Trypan Blue into the patient's eye.

A30. In an embodiment, a method of cataract extraction and treatment of glaucoma in an eye of a patient in need thereof, comprising:
i) instilling an ophthalmic composition comprising Trypan Blue into the patient's eye;
ii) surgically extracting the cataract of the Trypan Blue instilled eye;
iii) instilling an ophthalmic composition comprising Indigo Carmine into the cataract extracted eye; and
iv) surgically treating the glaucoma of the Indigo Carmine instilled eye.

A31. In an embodiment, a method of cataract extraction and treatment of glaucoma in an eye of a patient in need thereof, comprising:
i) instilling an ophthalmic composition comprising Indigo Carmine into the patient's eye;
ii) surgically extracting the cataract of the instilled eye; and
iii) surgically treating the glaucoma of the cataract extracted eye.

A32. In an embodiment, a method of cataract extraction and treatment of glaucoma in an eye of a patient in need thereof, comprising:
i) instilling an ophthalmic composition comprising Indigo Carmine and Trypan Blue into the patient's eye;
ii) surgically extracting the cataract of the instilled eye; and
iii) surgically treating the glaucoma of the cataract extracted eye.

A33. In an embodiment, a method of cataract extraction and treatment of glaucoma in an eye of a patient in need thereof, comprising:
i) instilling an ophthalmic composition comprising Indigo Carmine and an ophthalmic composition comprising Trypan Blue into the patient's eye;
ii) surgically extracting the cataract of the instilled eye; and
iii) surgically treating the glaucoma of the cataract extracted eye.

In certain embodiments, one or more than one (including for instance all) of the following further embodiments may comprise each of the other embodiments or parts thereof.

B1. The ophthalmic composition or method of any one of embodiments A1-A33, wherein the ophthalmic composition is an aqueous composition.

B2. The ophthalmic composition or method of any one of embodiments A1-A33 or B1, wherein the Indigo Carmine is present in an amount in the range of between approximately 0.001-0.4 wt. %, relative to the ophthalmic composition.

B3. The ophthalmic composition or method of any one of embodiments A1-A33 or B1-B2, wherein the Indigo Carmine is present in an amount in the range of between approximately 0.001-0.3 wt. %, between approximately 0.001-0.2 wt. %, between approximately 0.001-0.1 wt. %, between approximately 0.001-0.05 wt. %, between approximately 0.001-0.01 wt. %, between approximately 0.005-0.4 wt. %, between approximately 0.005-0.3 wt. %, between approximately 0.005-0.2 wt. %, between approximately 0.005-0.1 wt. %, between approximately 0.005-0.05 wt. %, between approximately 0.005-0.01 wt. %, between approximately 0.004-0.4 wt. %, between approximately 0.004-0.04 wt. %, between approximately 0.01-0.35 wt. %, between approximately 0.01-0.3 wt. %, between approximately 0.01-0.25 wt. %, between approximately 0.01-0.2 wt. %, between approximately 0.01-0.15 wt. %, between approximately 0.01-0.1 wt. %, between approximately 0.04-0.4 wt. %, between approximately 0.05-0.4 wt. %, between approximately 0.1-0.4 wt. %, between approximately 0.15-0.4 wt. %, between approximately 0.2-0.4 wt. %, between approximately 0.25-0.4 wt. %, between approximately 0.3-0.4 wt. %, between approximately 0.35-0.4 wt. %, between approximately 0.1-0.3 wt. %, between approximately 0.1-0.2 wt. %, between approximately 0.01-0.05 wt. %, or between approximately 0.05-0.1 wt. %, relative to the ophthalmic composition.

B4. The ophthalmic composition or method of any one of embodiments A1-A33 or B1-B3, wherein the Indigo Carmine is present in an amount of approximately 0.001 wt. %, approximately 0.002 wt. %, approximately 0.003 wt. %, approximately 0.004 wt. %, approximately 0.005 wt. %, approximately 0.006 wt. %, approximately 0.007 wt. %, approximately 0.008 wt. %, approximately 0.009 wt. %, approximately 0.01 wt. %, approximately 0.02 wt. %, approximately 0.03 wt. %, approximately 0.04 wt. %, approximately 0.05 wt. %, approximately 0.06 wt. %, approximately 0.07 wt. %, approximately 0.08 wt. %, approximately 0.09 wt. %, approximately 0.1 wt. %, approximately 0.2 wt. %, approximately 0.3 wt. %, or approximately 0.4 wt. %, relative to the ophthalmic composition.

B5. The ophthalmic composition or method of any one of embodiments A1-A33 or B1-B4, wherein the Indigo Carmine is present in an amount of at least 0.001 wt. %, at least 0.002 wt. %, at least 0.003 wt. %, at least 0.004 wt. %, at least 0.005 wt. %, at least 0.006 wt. %, at least 0.007 wt. %, at least 0.008 wt. %, at least 0.009 wt. %, at least 0.01 wt. %, at least 0.02 wt. %, at least 0.03 wt. %, at least 0.04 wt. %, at least 0.05 wt. %, at least 0.06 wt. %, at least 0.07 wt. %, at least 0.08 wt. %, at least 0.09 wt. %, at least 0.1 wt. %, at least 0.15 wt. %, at least 0.2 wt. %, at least 0.25 wt. %, at least 0.3 wt. %, or at least 0.35 wt. %, relative to the ophthalmic composition.

B6. The ophthalmic composition or method of any one of embodiments A1-A33 or B1-B5, wherein the ophthalmic composition further comprises Trypan Blue.

B7. The ophthalmic composition or method of any one of embodiments A1-A33 or B1-B6, wherein the Trypan Blue is present in an amount in the range of between approximately 0.001-0.1 wt. %, relative to the ophthalmic composition.

B8. The ophthalmic composition or method of any one of embodiments A1-A33 or B1-B7, wherein the Trypan Blue is present in an amount less than 0.1 wt. %, relative to the ophthalmic composition.

B9. The ophthalmic composition or method of any one of embodiments A1-A33 or B1-B8, wherein the Trypan Blue is present in an amount less than 0.05 wt. %, less than 0.04 wt. %, less than 0.03 wt. %, less than 0.02 wt. %, or less than 0.01 wt. %, relative to the ophthalmic composition.

B10. The ophthalmic composition or method of any one of embodiments A1-A33 or B1-B9, wherein the Trypan Blue is present in an amount in the range of between approximately 0.001-0.05 wt. %, between approximately 0.001-0.045 wt. %, between approximately 0.001-0.04 wt. %, between approximately 0.001-0.035 wt. %, between approximately 0.001-0.03 wt. %, between approximately 0.001-0.025 wt. %, between approximately 0.001-0.02 wt. %, between approximately 0.001-0.015 wt. %, between approximately 0.001-0.01 wt. %, between approximately 0.005-0.1 wt. %, between approximately 0.005-0.05 wt. %, between approximately 0.005-0.045 wt. %, between approximately 0.005-0.04 wt. %, between approximately 0.005-0.035 wt. %, between approximately 0.005-0.03 wt. %, between approximately 0.005-0.025 wt. %, between approximately 0.005-0.02 wt. %, between approximately 0.005-0.015 wt. %, between approximately 0.005-0.01 wt. %, between approximately 0.01-0.1 wt. %, between approximately 0.01-0.05 wt. %, between approximately 0.01-0.045 wt. %, between approximately 0.01-0.04 wt. %, between approximately 0.01-0.035 wt. %, between approximately 0.01-0.03 wt. %, between approximately 0.01-0.025 wt. %, between approximately 0.01-0.02 wt. %, between approximately 0.01-0.015 wt. %, or between approximately 0.02-0.04 wt. %, relative to the ophthalmic composition.

B11. The ophthalmic composition or method of any one of embodiments A1-A33 or B1-B10, wherein the Trypan Blue is present in an amount of approximately 0.001 wt. %, approximately 0.005 wt. %, approximately 0.01 wt. %, approximately 0.015 wt. %, approximately 0.02 wt. %, approximately 0.025 wt. %, approximately 0.03 wt. %, approximately 0.035 wt. %, approximately 0.04 wt. %, approximately 0.045 wt. %, approximately 0.05 wt. %, or approximately 0.1 wt. %, relative to the ophthalmic composition.

B12. The ophthalmic composition or method of any one of embodiments A1-A33 or B1-B11, wherein the ophthalmic composition comprises Indigo Carmine in an amount in the range of between approximately 0.001-0.4 wt. %, and the Trypan Blue in an amount in the range of between approximately 0.001-0.1 wt. %, relative to the ophthalmic composition, for example, comprises Indigo Carmine in an amount in the range of between approximately 0.005-0.3 wt. %, and Trypan Blue in an amount in the range of between approximately 0.005-0.05 wt. %, relative to the ophthalmic composition, such as comprises Indigo Carmine in an amount in the range of between approximately 0.005-0.3 wt. %, and Trypan Blue in an amount in the range of between approximately 0.005-0.045 wt. %, relative to the ophthalmic composition, comprises Indigo Carmine in an amount in the range of between approximately 0.005-0.3 wt. %, and Trypan Blue in an amount in the range of between approximately 0.005-0.04 wt. %, relative to the ophthalmic composition; or comprises Indigo Carmine in an amount of approximately 0.01 wt. %, and Trypan Blue in an amount of approximately 0.01 wt. %, relative to the ophthalmic composition.

B13. The ophthalmic composition or method of any one of embodiments A1-A33 or B1-B12, wherein the ophthalmic composition comprises 0.4 wt. % of Indigo Carmine and 0.06 wt. % Trypan Blue, relative to the ophthalmic composition.

B14. The ophthalmic composition or method of any one of embodiments A1-A33 or B1-B13, wherein an ophthalmic composition comprising Indigo Carmine (for example, Indigo Carmine in an amount in the range of between approximately 0.001-0.4 wt. %, such as 0.4 wt. % of Indigo Carmine, relative to the ophthalmic composition) is co-administered with an ophthalmic composition comprising Trypan Blue (for example, Trypan Blue in an amount in the range of between approximately 0.001-0.1 wt. %, such as 0.06 wt. % Trypan Blue, relative to the ophthalmic composition), wherein the co-administration of the ophthalmic composition comprising Trypan Blue is as at the same time, or sequentially before or after.

B15. The ophthalmic composition or method of any one of embodiments A1-A33 or B1-B14, wherein the ophthalmic composition further comprises Brilliant Blue.

B16. The ophthalmic composition or method of any one of embodiments A1-A33 or B1-B15, wherein the ophthalmic composition further comprises Patent Blue.

B17. The ophthalmic composition or method of any one of embodiments A1-A33 or B1-B16, wherein the ophthalmic composition further comprises Indocyanine Green.

B18. The ophthalmic composition or method of any one of embodiments A1-A33 or B1-B17, wherein the ophthalmic composition further comprises Fluorescein.

B19. The ophthalmic composition or method of any one of embodiments A1-A33 or B1-B18, wherein the ophthalmic composition is an injectable ophthalmic formulation.

B20. The ophthalmic composition or method of any one of embodiments A1-A33 or B1-B19, wherein the ophthalmic composition is a sterile aqueous solution.

B21. The ophthalmic composition or method of any one of embodiments A1-A33 or B1-B20, wherein the ophthalmic composition comprises or is an ophthalmic irrigation solution.

B22. The ophthalmic composition or method of any one of embodiments A1-A33 or B1-B21, wherein the ophthalmic irrigation solution may be a balanced salt solution (BSS), a Balanced Salt Solution Plus (BSS Plus®), an Alsever's salt solution, an Earle's balanced salt solution (EBSS), a Gey's balanced salt solution (GBSS), a Hanks' balanced salt solution (MSS), a Dulbecco's phosphate buffered saline (PBS), a Puck's balanced salt solution, a Ringer's balanced salt solution (RBSS), a Simm's balanced salt solution (SBSS), a TRIS-buffered saline (TBS), or a Tyrode's balanced salt solution solution (TBSS), or combinations thereof.

B23. The ophthalmic composition or method of any one of embodiments A1-A33 or B1-B22, wherein the ophthalmic composition comprises sugar compounds, such as glucose or dextrose.

B24. The ophthalmic composition or method of any one of embodiments A1-A33 or B1-B23, wherein the ophthalmic composition comprises anti-oxidant compounds, such as glutathione.

B25. The ophthalmic composition or method of any one of embodiments A1-A33 or B1-B24, wherein the ophthalmic composition is an isotonic, aqueous solution, such as an isotonic, sterile, aqueous solution, having a neutral pH, such as a between pH 6-8, between pH 6.5-7.5, between pH 7-7.6, between pH 7.3-7.6, or between pH 6.8-7.2, or about pH 7.

B26. The ophthalmic composition or method of any one of embodiments A1-A33 or B1-B25, wherein the ophthalmic composition comprises sodium, potassium, calcium, and/or magnesium cations.

B27. The ophthalmic composition or method of any one of embodiments A1-A33 or B1-B26, wherein the ophthalmic composition comprises phosphate ion, mono-hydrogen phosphate ion, di-hydrogen phosphate ion, citrate ion, bicarbonate, or chloride ion, or combinations thereof.

B28. The ophthalmic composition or method of any one of embodiments A1-A33 or B1-B27, wherein the ophthalmic composition comprises inorganic salts and/or organic salts.

B29. The ophthalmic composition or method of any one of embodiments A1-A33 or B1-B28, wherein the inorganic salts and/or organic salts comprises sodium chloride, potassium chloride, calcium chloride, magnesium chloride, sodium acetate, sodium phosphate, sodium mono-hydrogen phosphate (sodium mono-hydrogen orthophosphate), sodium di-hydrogen phosphate (sodium di-hydrogen orthophosphate), sodium bicarbonate, or sodium citrate, or combinations thereof.

B30. The ophthalmic composition or method of any one of embodiments A1-A33 or B1-B29, wherein the ophthalmic composition has an osmolality between 200-400 mosmol/kg, such as 250-350 mosmol/kg, 300-350 mosmol/kg, or 250-325 mosmol/kg, for example, 200 mosmol/kg, 250 mosmol/kg, 275 mosmol/kg, 300 mosmol/kg, or 325 mosmol/kg, such as 300 mosmol/kg.

B31. The ophthalmic composition or method of any one of embodiments A1-A33 or B1-B30, wherein the ophthalmic composition further comprises one or more additional ophthalmically acceptable excipients and additives.

B32. The ophthalmic composition or method of any one of embodiments A1-A33 or B1-B31, wherein the ophthalmic composition is used for application to an eye.

B33. The ophthalmic composition or method of any one of embodiments A1-A33 or B1-B32, wherein the application of the ophthalmic composition to the eye is via topical application to said eye.

B34. The ophthalmic composition or method of any one of embodiments A1-A33 or B1-B33, wherein the application of the ophthalmic composition to the eye is via injection into said eye.

B35. The ophthalmic composition or method of any one of embodiments A1-A33 or B1-B34, wherein the application of the ophthalmic composition to the eye is via injection into the anterior chamber of said eye.

B36. The ophthalmic composition or method of any one of embodiments A1-A33 or B1-B35, wherein the eye is a glaucomatous eye.

B37. The ophthalmic composition or method of any one of embodiments A1-A33 or B1-B36, wherein the eye has a cataract.

B38. The ophthalmic composition or method of any one of embodiments A1-A33 or B1-B37, wherein the method is a method for ocular surgery in a patient in need thereof, and wherein said method comprises instilling the ophthalmic composition of any one of the above embodiments and any one or more of the further embodiments.

B39. The ophthalmic composition or method of any one of embodiments A1-A33 or B1-B38, wherein the method includes an ocular surgery, or the ocular surgery is, selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique.

B40. The ophthalmic composition or method of any one of embodiments A1-A33 or B1-B39, wherein the method, or the ocular surgery, includes a combination of two or more of the following ocular surgeries selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique.

B41. The ophthalmic composition or method of any one of embodiments A1-A33 or B1-B40, wherein the ocular surgery is or includes glaucoma surgery.

B42. The ophthalmic composition or method of any one of embodiments A1-A33 or B1-B41, wherein the ocular surgery is or includes minimally invasive glaucoma surgery (MIGS).

B43. The ophthalmic composition or method of any one of embodiments A1-A33 or B1-B42, wherein the ocular surgery is or includes cataract surgery.

B44. The ophthalmic composition or method of any one of embodiments A1-A33 or B1-B43, wherein the ocular surgery is or includes retinal surgery.

B45. The ophthalmic composition or method of any one of embodiments A1-A33 or B1-B44, wherein the ocular surgery is or includes lens replacement surgery.

B46. The ophthalmic composition or method of any one of embodiments A1-A33 or B1-B45, wherein the ocular surgery is or includes surgery to treat ocular trauma.

B47. The ophthalmic composition or method of any one of embodiments A1-A33 or B1-B46, wherein the ocular surgery is or includes refractive lensectomy.

B48. The ophthalmic composition or method of any one of embodiments A1-A33 or B1-B47, wherein the ocular surgery is or includes corneal surgery.

B49. The ophthalmic composition or method of any one of embodiments A1-A33 or B1-B48, wherein the ocular surgery comprises extracting a cataract and treating glaucoma.

B50. The ophthalmic composition or method of any one of embodiments A1-A33 or B1-B49, wherein the ocular surgery is or includes endothelial keratoplasty.

B51. The ophthalmic composition or method of any one of embodiments A1-A33 or B1-B50, wherein the ocular surgery is or includes Descemet's Membrane Endothelial Keratoplasty (DMEK).

B52. The ophthalmic composition or method of any one of embodiments A1-A33 or B1-B51, wherein the ocular surgery is or includes capsulorhexis.

B53. The ophthalmic composition or method of any one of embodiments A1-A33 or B1-B52, wherein the ocular surgery is or includes lamellar corneal transplantation.

B54. The ophthalmic composition or method of any one of embodiments A1-A33 or B1-B53, wherein the ocular surgery is or includes a minimally invasive corneal procedure.

B55. The ophthalmic composition or method of any one of embodiments A1-A33 or B1-B54, wherein the ocular surgery is or includes a minimally invasive corneal procedure corrects refractive error.

B56. The ophthalmic composition or method of any one of embodiments A1-A33 or B1-B55, wherein the ocular surgery is or includes a corneal refractive procedure.

B57. The ophthalmic composition or method of any one of embodiments A1-A33 or B1-B56, wherein the ocular surgery is or includes a small incision lenticule extraction (SMILE).

B58. The ophthalmic composition or method of any one of embodiments A1-A33 or B1-B57, wherein the ocular surgery is or includes an Ab interno Canaloplasty (ABiC).

B59. The ophthalmic composition or method of any one of embodiments A1-A33 or B1-B58, wherein the ocular surgery is or includes an Ab externo Canaloplasty (ABeC).

B60. The ophthalmic composition or method of any one of embodiments A1-A33 or B1-B59, wherein the ocular surgery is or includes an ocular surface diagnostic technique.

B61. The ophthalmic composition or method of any one of embodiments A1-A33 or B1-B60, wherein the ocular surgery is a combination of glaucoma surgery and cataract surgery.

B62. The ophthalmic composition or method of any one of embodiments A1-A33 or B1-B61, wherein the ocular surgery is a combination of minimally invasive glaucoma surgery (MIGS) and cataract surgery.

B63. The ophthalmic composition or method of any one of embodiments A1-A33 or B1-B62, wherein the ocular surgery is a combination of minimally invasive glaucoma surgery (MIGS) and endothelial keratoplasty.

B64. The ophthalmic composition or method of any one of embodiments A1-A33 or B1-B63, wherein the ocular surgery is a combination of endothelial keratoplasty and cataract surgery.

B65. The ophthalmic composition or method of any one of embodiments A1-A33 or B1-B64, wherein the patient's eye is glaucomatous.

B66. The ophthalmic composition or method of any one of embodiments A1-A33 or B1-B65, wherein the patient's eye has a cataract.

B67. The ophthalmic composition or method of any one of embodiments A1-A33 or B1-B66, wherein the ophthalmic composition is instilled into the eye by injection.

B68. The ophthalmic composition or method of any one of embodiments A1-A33 or B1-B67, wherein the ophthalmic composition is instilled into the eye by injection into the anterior chamber of the eye.

B69. The ophthalmic composition or method of any one of embodiments A1-A33 or B1-B68, wherein the ophthalmic composition is instilled into the eye by a plurality of injections.

B70. The ophthalmic composition or method of any one of embodiments A1-A33 or B1-B69, wherein the ophthalmic composition is instilled into the eye by a plurality of injections into the anterior chamber of the eye.

B71. The ophthalmic composition or method of any one of embodiments A1-A33 or B1-B70, wherein the method identifies, marks, or stains an intraocular structure(s) or membrane(s) within the patient's eye in a visually identifiable manner.

B72. The ophthalmic composition or method of any one of embodiments A1-A33 or B1-B71, wherein the method identifies, marks, or stains an intraocular structure(s) or membrane(s) within the patient's eye in a visually identifiable manner easily visible by the naked eye of a surgeon.

B73. The ophthalmic composition or method of any one of embodiments A1-A33 or B1-B72, wherein the intraocular structure(s) or membrane(s) within the patient's eye is identified, marked, or stained, in a visually identifiable manner.

B74. The ophthalmic composition or method of any one of embodiments A1-A33 or B1-B73, wherein a plurality of intraocular structures or membranes within the eye are identified, marked, or stained, in a visually identifiable manner.

B75. The ophthalmic composition or method of any one of embodiments A1-A33 or B1-B74, wherein the method facilitates ready identification of the intraocular structure(s) or membrane(s) within the eye.

B76. The ophthalmic composition or method of any one of embodiments A1-A33 or B1-B75, wherein the method identifies, marks, or stains a portion of the intraocular structure(s) or membrane(s) within the patient's eye.

B77. The ophthalmic composition or method of any one of embodiments A1-A33 or B1-B76, wherein the method identifies, marks, or stains a plurality of the intraocular structures or membranes within the patient's eye.

B78. The ophthalmic composition or method of any one of embodiments A1-A33 or B1-B77, wherein the identified, marked, or stained intraocular structure(s) or membrane(s) within the patient's eye is selected from a group consisting of: a fine vessel, an aqueous vein, an episcleral vein, a collector channel, a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

B79. The ophthalmic composition or method of any one of embodiments A1-A33 or B1-B78, wherein the identified, marked, or stained intraocular structure(s) or membrane(s) within the patient's eye is a fine vessel.

B80. The ophthalmic composition or method of any one of embodiments A1-A33 or B1-B79, wherein the identified, marked, or stained intraocular structure(s) or membrane(s) within the patient's eye is a fine vessel is in the conjunctiva adjacent to the limbus.

B81. The ophthalmic composition or method of any one of embodiments A1-A33 or B1-B80, wherein the identified, marked, or stained intraocular structure(s) or membrane(s) within the patient's eye is an aqueous vein.

B82. The ophthalmic composition or method of any one of embodiments A1-A33 or B1-B81, wherein the identified, marked, or stained intraocular structure(s) or membrane(s) within the patient's eye is an episcleral vein.

B83. The ophthalmic composition or method of any one of embodiments A1-A33 or B1-B82, wherein the identified, marked, or stained intraocular structure(s) or membrane(s) within the patient's eye is a collector channel.

B84. The ophthalmic composition or method of any one of embodiments A1-A33 or B1-B83, wherein the identified, marked, or stained intraocular structure(s) or membrane(s) within the patient's eye is a collector channel/aqueous/episcleral vein system.

B85. The ophthalmic composition or method of any one of embodiments A1-A33 or B1-B84, wherein the identified, marked, or stained intraocular structure(s) or membrane(s) within the patient's eye is an aqueous drainage system of said eye.

B86. The ophthalmic composition or method of any one of embodiments A1-A33 or B1-B85, wherein the identified, marked, or stained intraocular structure(s) or membrane(s) within the patient's eye is conventional drainage system of said eye.

B87. The ophthalmic composition or method of any one of embodiments A1-A33 or B1-B86, wherein the identified, marked, or stained intraocular structure(s) or membrane(s) within the patient's eye is a conjunctival venous system.

B88. The ophthalmic composition or method of any one of embodiments A1-A33 or B1-B87, wherein the identified, marked, or stained intraocular structure(s) or membrane(s) within the patient's eye is a subconjunctival space.

B89. The ophthalmic composition or method of any one of embodiments A1-A33 or B1-B88, wherein the identified, marked, or stained intraocular structure(s) or membrane(s) within the patient's eye is a deep scleral plexus.

B90. The ophthalmic composition or method of any one of embodiments A1-A33 or B1-B89, wherein the identified, marked, or stained intraocular structure(s) or membrane(s) within the patient's eye is a deep scleral plexus visually identifiable once the conjunctiva is reflected away.

B91. The ophthalmic composition or method of any one of embodiments A1-A33 or B1-B90, wherein the identified, marked, or stained intraocular structure(s) or membrane(s) within the patient's eye is a trabecular meshwork.

B92. The ophthalmic composition or method of any one of embodiments A1-A33 or B1-B91, wherein the identified, marked, or stained intraocular structure(s) or membrane(s) within the patient's eye is a posterior aspect of a trabecular meshwork.

B93. The ophthalmic composition or method of any one of embodiments A1-A33 or B1-B92, wherein the identified, marked, or stained intraocular structure(s) or membrane(s) within the patient's eye is a canal of Schlemm.

B94. The ophthalmic composition or method of any one of embodiments A1-A33 or B1-B93, wherein the identified, marked, or stained intraocular structure(s) or membrane(s) within the patient's eye is a suprachoroidal space.

B95. The ophthalmic composition or method of any one of embodiments A1-A33 or B1-B94, wherein the identified, marked, or stained intraocular structure(s) or membrane(s) within the patient's eye is a scleral spur.

B96. The ophthalmic composition or method of any one of embodiments A1-A33 or B1-B95, wherein the identified, marked, or stained intraocular structure(s) or membrane(s) within the patient's eye is the anterior capsule of a crystalline lens.

B97. The ophthalmic composition or method of any one of embodiments A1-A33 or B1-B96, wherein the identified, marked, or stained intraocular structure(s) or membrane(s) within the patient's eye is a cornea.

B98. The ophthalmic composition or method of any one of embodiments A1-A33 or B1-B97, wherein the identified, marked, or stained intraocular structure(s) or membrane(s) within the patient's eye is a lens capsule.

B99. The ophthalmic composition or method of any one of embodiments A1-A33 or B1-B98, wherein the identified, marked, or stained intraocular structure(s) or membrane(s) within the patient's eye is a retinal membrane.

B100. The ophthalmic composition or method of any one of embodiments A1-A33 or B1-B99, wherein the identified, marked, or stained intraocular structure(s) or membrane(s) within the patient's eye is a corneal endothelial membrane.

B101. The ophthalmic composition or method of any one of embodiments A1-A33 or B1-B100, wherein the identified, marked, or stained intraocular structure(s) or membrane(s) within the patient's eye is a Descemet's membrane.

B102. The ophthalmic composition or method of any one of embodiments A1-A33 or B1-B101, wherein the Indigo Carmine of the ophthalmic composition identifies, marks, or stains a trabecular meshwork and a canal of Schlemm in the patient's eye.

B103. The ophthalmic composition or method of any one of embodiments A1-A33 or B1-B102, wherein the Indigo Carmine of the ophthalmic composition identifies, marks, or stains a canal of Schlemm more than a trabecular meshwork in the patient's eye.

B104. The ophthalmic composition or method of any one of embodiments A1-A33 or B1-B103, wherein the Indigo Carmine of the ophthalmic composition identifies, marks, or stains a trabecular meshwork less than a canal of Schlemm in the patient's eye.

B105. The ophthalmic composition or method of any one of embodiments A1-A33 or B1-B104, wherein the method facilitates diagnosis of the patient's eye.

B106. The ophthalmic composition or method of any one of embodiments A1-A33 or B1-B105, wherein the method facilitates diagnosis of the intraocular structure(s) or membrane(s) within the patient's eye.

B107. The ophthalmic composition or method of any one of embodiments A1-A33 or B1-B106, wherein the method facilitates an ocular surgeon's diagnosis of fluid flow and drainage of the patient's eye during the ocular surgery.

B108. The ophthalmic composition or method of any one of embodiments A1-A33 or B1-B107, wherein the method facilitates treatment of the patient's eye.

B109. The ophthalmic composition or method of any one of embodiments A1-A33 or B1-B108, wherein the method facilitates surgical treatment of the patient's eye.

B110. The ophthalmic composition or method of any one of embodiments A1-A33 or B1-B109, wherein the method facilitates surgical treatment of the identified intraocular structure(s) or membrane(s) within the eye.

B111. The ophthalmic composition or method of any one of embodiments A1-A33 or B1-B110, wherein the method facilitates surgical removal of the identified intraocular structure(s) or membrane(s) within the eye.

B112. The ophthalmic composition or method of any one of embodiments A1-A33 or B1-B111, wherein the method facilitates extracting a cataract and treating glaucoma.

B113. The ophthalmic composition or method of any one of embodiments A1-A33 or B1-B112, wherein the method further comprises introducing an ophthalmic device into the instilled eye.

B114. The ophthalmic composition or method of any one of embodiments A1-A33 or B1-B113, wherein the instilled ophthalmic composition facilitates accurate and/or precise inserting, placement, positioning, repositioning, lifting, and/or removal, of an ophthalmic device within the patient's eye.

B115. The ophthalmic composition or method of any one of embodiments A1-A33 or B1-B114, wherein the method facilitates accurate and/or precise inserting, placement, positioning, repositioning, lifting, and/or removal, of an ophthalmic device proximate the identified intraocular structure(s) or membrane(s) within the patient's eye.

B116. The ophthalmic composition or method of any one of embodiments A1-A33 or B1-B115, wherein the method facilitates accurate and/or precise inserting, placement, positioning, repositioning, lifting, and/or removal, of a plurality of ophthalmic devices within the Indigo Carmine instilled patient's eye.

B117. The ophthalmic composition or method of any one of embodiments A1-A33 or B1-B116, wherein the method facilitates accurate and/or precise inserting of an ophthalmic device proximate the identified intraocular structure(s) or membrane(s) within the patient's eye.

B118. The ophthalmic composition or method of any one of embodiments A1-A33 or B1-B117, wherein the method facilitates accurate and/or precise placement of an ophthalmic device proximate the identified intraocular structure(s) or membrane(s) within the patient's eye.

B119. The ophthalmic composition or method of any one of embodiments A1-A33 or B1-B118, wherein the method facilitates accurate and/or precise positioning of an ophthalmic device proximate the identified intraocular structure(s) or membrane(s) within the patient's eye.

B120. The ophthalmic composition or method of any one of embodiments A1-A33 or B1-B119, wherein the method facilitates accurate and/or precise repositioning of an ophthalmic device proximate the identified intraocular structure(s) or membrane(s) within the patient's eye.

B121. The ophthalmic composition or method of any one of embodiments A1-A33 or B1-B120, wherein the method facilitates accurate and/or precise removal of an ophthalmic device proximate the identified intraocular structure(s) or membrane(s) within the patient's eye.

B122. The ophthalmic composition or method of any one of embodiments A1-A33 or B1-B121, wherein the ophthalmic device is a stent.

B123. The ophthalmic composition or method of any one of embodiments A1-A33 or B1-B122, wherein the method facilitates an ocular surgeon's determination of the type of stent to utilize during the ocular surgery.

B124. The ophthalmic composition or method of any one of embodiments A1-A33 or B1-B123, wherein the method facilitates an ocular surgeon's placement of the stent during the ocular surgery.

B125. The ophthalmic composition or method of any one of embodiments A1-A33 or B1-B124, wherein the method facilitates an ocular surgeon's determination of the type of stent to utilize and the placement of the stent during the ocular surgery.

B126. The ophthalmic composition or method of any one of embodiments A1-A33 or B1-B125, wherein the ophthalmic device is a glaucoma stent.

B127. The ophthalmic composition or method of any one of embodiments A1-A33 or B1-B126, wherein the ophthalmic device is a suprachoroidal stent.

B128. The ophthalmic composition or method of any one of embodiments A1-A33 or B1-127, wherein the ophthalmic device is an intraocular lens during cataract surgery.

B129. The ophthalmic composition or method of any one of embodiments A1-A33 or B1-B128, wherein the ophthalmic device is introduced proximate to canal of Schlemm of the patient's eye.

B130. The ophthalmic composition or method of any one of embodiments A1-A33 or B1-B129, wherein the ophthalmic device is inserted into the canal of Schlemm of the patient's eye.

B131. The ophthalmic composition or method of any one of embodiments A1-A33 or B1-B130, wherein the ophthalmic device is inserted into the suprachorodial space of the patient's eye.

B132. The ophthalmic composition or method of any one of embodiments A1-A33 or B1-B131, wherein the ophthalmic device is pre-treated with Indigo Carmine.

B133. The ophthalmic composition or method of any one of embodiments A1-A33 or B1-B132, wherein the ophthalmic device is pre-treated with Trypan Blue.

B134. The ophthalmic composition or method of any one of embodiments A1-A33 or B1-B133, wherein the method further comprises instilling an ophthalmic composition comprising Trypan Blue.

B135. The ophthalmic composition or method of any one of embodiments A1-A33 or B1-B134, wherein the instilled the ophthalmic composition comprises both Indigo Carmine and Trypan Blue.

B136. The ophthalmic composition or method of any one of embodiments A1-A33 or B1-B135, wherein intraocular structures or membranes of the instilled eye are identified, marked, or stained, by Indigo Carmine prior to extracting of the cataract.

B137. The ophthalmic composition or method of any one of embodiments A1-A33 or B1-B136, wherein intraocular structures or membranes of the instilled eye are identified, marked, or stained, by both Indigo Carmine and Trypan Blue prior to extracting of the cataract.

B138. The ophthalmic composition or method of any one of embodiments A1-A33 or B1-B137, wherein the surgical treatment of the glaucoma in said Indigo Carmine instilled eye comprises introducing an ophthalmic device into said eye.

B139. The ophthalmic composition or method of any one of embodiments A1-A33 or B1-B138, wherein the surgical treatment of the glaucoma in said Indigo Carmine instilled eye comprises: a) visually identifying an Indigo Carmine stained canal of Schlemm; and b) introducing an ophthalmic device into the patient's eye proximate the Indigo Carmine stained canal of Schlemm.

B140. The ophthalmic composition or method of any one of embodiments A1-A33 or B1-B139, wherein the Indigo Carmine containing ophthalmic composition and the Trypan Blue containing ophthalmic composition are co-instilled concurrently, co-instilled sequentially with instilling of the Indigo Carmine containing ophthalmic composition followed by the Trypan Blue containing ophthalmic composition, or co-instilled sequentially with instilling of the Trypan Blue containing ophthalmic composition followed by the Indigo Carmine containing ophthalmic composition.

B141. The ophthalmic composition or method of any one of embodiments A1-A33 or B1-B140, wherein the ophthalmic composition is instilled into the patient's eye over a period of time in the range of between 1 second to 2 minutes.

B142. The ophthalmic composition or method of any one of embodiments A1-A33 or B1-B141, wherein the ophthalmic composition is instilled into the patient's eye over a period of at least 10 seconds, at least 20 seconds, at least 30 seconds, at least 45 seconds, at least 1 minute, or at least 1.5 minutes.

B143. The ophthalmic composition or method of any one of embodiments A1-A33 or B1-B142, wherein the ophthalmic composition is instilled into the patient's eye over a period of time until the composition egresses from one or more channels in the patient's eye.

B144. The ophthalmic composition or method of any one of embodiments A1-A33 or B1-B143, wherein a plurality of the instillations of the ophthalmic composition is conducted over a period of time until at least 25%, at least 50%, at least 75%, at least 90%, or at least 95%, of the canal of Schlemm is visually identifiable.

B145. The ophthalmic composition or method of any one of embodiments A1-A33 or B1-B144, wherein the method results in reduced surgical manipulation, relative to an ocular surgery not using said ophthalmic composition.

B146. The ophthalmic composition or method of any one of embodiments A1-A33 or B1-B145, wherein the method results in reduced tissue manipulation, relative to an ocular surgery not using said ophthalmic composition.

B147. The ophthalmic composition or method of any one of embodiments A1-A33 or B1-B146, wherein the method results in less severe adverse side effects, relative to an ocular surgery not using said ophthalmic composition.

C1. The ophthalmic composition or method of any one of embodiments A1-A33 or B1-B147, wherein the method facilitates accurate and/or precise inserting, placement, positioning, repositioning, lifting, and/or removal, of a plurality of ophthalmic devices within the Indigo Carmine instilled patient's eye, wherein the plurality of ophthalmic devices are positioned around the circumference of the drainage angle.

C2. The ophthalmic composition or method of any one of embodiments A1-A33, B1-B147, or C1, wherein the method facilitates accurate and/or precise inserting, placement, positioning, repositioning, lifting, and/or removal, of a plurality of ophthalmic devices within the Indigo Carmine instilled patient's eye, wherein the plurality of ophthalmic devices are positioned in a plurality of locations around the circumference of the drainage angle.

C3. The ophthalmic composition or method of any one of embodiments A1-A33, B1-B147, or C1-C2, wherein upon identifying the ocular drainage structures an ophthalmic device is placed proximate or adjacent to a plurality, or each, of the identified ocular drainage structures.

C4. The ophthalmic composition or method of any one of embodiments A1-A33, B1-B147, or C1-C3, wherein the plurality of ophthalmic devices are positioned in, near or adjacent to the ocular drainage structures identified in the instilled patient's eye around the circumference of the drainage angle.

C5. The ophthalmic composition or method of any one of embodiments A1-A33, B1-B147, or C1-C4, wherein the plurality of ophthalmic devices are positioned in, near or adjacent to an ocular drainage structure identified in the instilled patient's eye around the circumference of the drainage angle.

C6. The ophthalmic composition or method of any one of embodiments A1-A33, B1-B147, or C1-C5, wherein the plurality of ophthalmic devices are positioned in, near or adjacent to 2 ocular drainage structures identified in the instilled patient's eye around the circumference of the drainage angle.

C7. The ophthalmic composition or method of any one of embodiments A1-A33, B1-B147, or C1-C6, wherein the plurality of ophthalmic devices positioned around the circumference of the drainage angle are placed in, proximate, or adjacent to at least 2 of the ocular drainage structures identified in the instilled patient's eye.

C8. The ophthalmic composition or method of any one of embodiments A1-A33, B1-B147, or C1-C7, wherein the plurality of ophthalmic devices positioned around the circumference of the drainage angle are placed in, proximate, or adjacent to at least 2 or at least 3 of the ocular drainage structures identified in the instilled patient's eye.

C9. The ophthalmic composition or method of any one of embodiments A1-A33, B1-B147, or C1-C8, wherein the plurality of ophthalmic devices are positioned in, near or adjacent to 3 ocular drainage structures identified in the instilled patient's eye around the circumference of the drainage angle.

C10. The ophthalmic composition or method of any one of embodiments A1-A33, B1-B147, or C1-C9, wherein the plurality of ophthalmic devices positioned around the circumference of the drainage angle are placed in, proximate, or adjacent to at least 3 of the ocular drainage structures identified in the instilled patient's eye.

C11. The ophthalmic composition or method of any one of embodiments A1-A33, B1-B147, or C1-C10, wherein the plurality of ophthalmic devices are positioned in, near or adjacent to 4 ocular drainage structures identified in the instilled patient's eye around the circumference of the drainage angle.

C12. The ophthalmic composition or method of any one of embodiments A1-A33, B1-B147, or C1-C11, wherein the plurality of ophthalmic devices are positioned in, near or adjacent to 5 ocular drainage structures identified in the instilled patient's eye around the circumference of the drainage angle.

C13. The ophthalmic composition or method of any one of embodiments A1-A33, B1-B147, or C1-C12, wherein the plurality of ophthalmic devices are positioned in, near or adjacent to between 2-20 ocular drainage structures, 3-20 ocular drainage structures, 4-20 ocular drainage structures, 5-20 ocular drainage structures, 2-10 ocular drainage structures, 3-10 ocular drainage structures, 4-10 ocular drainage structures, 5-10 ocular drainage structures, 2-5 ocular drainage structures, or 3-5 ocular drainage structures, identified in the instilled patient's eye around the circumference of the drainage angle.

C14. The ophthalmic composition or method of any one of embodiments A1-A33, B1-B147, or C1-C13, wherein the plurality of ophthalmic devices are positioned around the circumference of the drainage angle such that each ophthalmic device is placed in, proximate, or adjacent to, an identified ocular drainage structure.

C15. The ophthalmic composition or method of any one of embodiments A1-A33, B1-B147, or C1-C14, wherein the plurality of ophthalmic devices are placed in, proximate, or adjacent to, one or more identified ocular drainage structures.

C16. The ophthalmic composition or method of any one of embodiments A1-A33, B1-B147, or C1-C15, wherein the plurality of ophthalmic devices are placed in, proximate, or adjacent to, an equal plurality of the identified ocular drainage structures.

C17. The ophthalmic composition or method of any one of embodiments A1-A33, B1-B147, or C1-C16, wherein a plurality of ophthalmic devices are distributed around the circumference of the drainage angle and placed in, proximate, or adjacent to, identified ocular drainage structures in said instilled eye.

C18. The ophthalmic composition or method of any one of embodiments A1-A33, B1-B147, or C1-C17, wherein a plurality of ophthalmic devices are distributed around the circumference of the drainage angle and placed in, proximate, or adjacent to, an equal plurality of identified ocular drainage structures in said instilled eye.

C19. The ophthalmic composition or method of any one of embodiments A1-A33, B1-B147, or C1-C18, wherein a plurality of ophthalmic devices are distributed around the circumference of the drainage angle and placed in, proximate, or adjacent to, a less than equal plurality of plurality of identified ocular drainage structures in said instilled eye.

C20. The ophthalmic composition or method of any one of embodiments A1-A33, B1-B147, or C1-C19, wherein the plurality of ophthalmic devices are positioned in, near or adjacent to 2, 3, 4, 5, 6, 7, 8, 9, 10, 11, 12, 13, 14, 15, 16, 17, 18, 19, or 20 ocular drainage structures, identified in the instilled patient's eye around the circumference of the drainage angle.

C21. The ophthalmic composition or method of any one of embodiments A1-A33, B1-B147, or C1-C20, wherein the plurality of ophthalmic devices are positioned in, near or adjacent to 2, 3, 4, 5, 6, 7, 8, 9, or 10 ocular drainage structures, identified in the instilled patient's eye around the circumference of the drainage angle.

C22. The ophthalmic composition or method of any one of embodiments A1-A33, B1-B147, or C1-C21, wherein the plurality of ophthalmic devices are positioned in, near or adjacent to 3, 4, 5, 6, 7, 8, 9, 10, 11, 12, 13, 14, 15, 16, 17, 18, 19, or 20 ocular drainage structures, identified in the instilled patient's eye around the circumference of the drainage angle.

C23. The ophthalmic composition or method of any one of embodiments A1-A33, B1-B147, or C1-C22, wherein the plurality of ophthalmic devices are positioned in, near or adjacent to 3, 4, 5, 6, 7, 8, 9, or 10 ocular drainage structures, identified in the instilled patient's eye around the circumference of the drainage angle.

C24. The ophthalmic composition or method of any one of embodiments A1-A33, B1-B147, or C1-C23, wherein the plurality of ophthalmic devices are positioned in, near or adjacent to 3, 4, 5, 6, 7, 8, or 9 ocular drainage structures, identified in the instilled patient's eye around the circumference of the drainage angle.

C25. The ophthalmic composition or method of any one of embodiments A1-A33, B1-B147, or C1-C24, wherein between 1-5, between 2-5, between 2-4, or between 1-3 ophthalmic devices of the plurality of ophthalmic devices are positioned in, near or adjacent to the ocular drainage structures, identified in the instilled patient's eye around the circumference of the drainage angle.

C26. The ophthalmic composition or method of any one of embodiments A1-A33, B1-B147, or C1-C25, wherein between 2-5 ophthalmic devices of the plurality of ophthalmic devices are positioned in, near or adjacent to the ocular drainage structures, identified in the instilled patient's eye around the circumference of the drainage angle.

C27. The ophthalmic composition or method of any one of embodiments A1-A33, B1-B147, or C1-C26, wherein the plurality of ophthalmic devices is 2, 3, 4, 5, or 6 ophthalmic devices.

C28. The ophthalmic composition or method of any one of embodiments A1-A33, B1-B147, or C1-C27, wherein the plurality of ophthalmic devices is 3, 4, 5, or 6 ophthalmic devices.

C29. The ophthalmic composition or method of any one of embodiments A1-A33, B1-B147, or C1-C28, wherein the plurality of ophthalmic devices is 3 ophthalmic devices.

C30. The ophthalmic composition or method of any one of embodiments A1-A33, B1-B147, or C1-C29, wherein the plurality of ophthalmic devices is 4 ophthalmic devices.

C31. The ophthalmic composition or method of any one of embodiments A1-A33, B1-B147, or C1-C30, wherein the plurality of ophthalmic devices is 5 ophthalmic devices.

C32. The ophthalmic composition or method of any one of embodiments A1-A33, B1-B147, or C1-C31, wherein the plurality of ophthalmic devices is 6 ophthalmic devices.

C33. The ophthalmic composition or method of any one of embodiments A1-A33, B1-B147, or C1-C32, wherein the plurality of ophthalmic devices is 7 ophthalmic devices.

C34. The ophthalmic composition or method of any one of embodiments A1-A33, B1-B147, or C1-C33, wherein the plurality of ophthalmic devices is 8 ophthalmic devices.

C35. The ophthalmic composition or method of any one of embodiments A1-A33, B1-B147, or C1-C34, wherein the plurality of ophthalmic devices is 9 ophthalmic devices.

C36. The ophthalmic composition or method of any one of embodiments A1-A33, B1-B147, or C1-C35, wherein the plurality of ophthalmic devices is 10 ophthalmic devices.

C37. The ophthalmic composition or method of any one of embodiments A1-A33, B1-B147, or C1-C36, wherein 2, 3, 4, 5, or 6 ophthalmic devices are placed in, proximate, or adjacent to, 2, 3, 4, 5, or 6 of the identified ocular drainage structures, respectively.

C38. The ophthalmic composition or method of any one of embodiments A1-A33, B1-B147, or C1-37, wherein 3, 4, 5, or 6 ophthalmic devices are placed in, proximate, or adjacent to, 3, 4, 5, or 6 of the identified ocular drainage structures, respectively.

C39. The ophthalmic composition or method of any one of embodiments A1-A33, B1-B147, or C1-C38, wherein the ocular drainage structure identified in the instilled patient's eye around the circumference of the drainage angle is selected from the group consisting of: a fine vessel, an aqueous vein, an episcleral vein, an interscleral channel (e.g., collector channel), a collector channel, a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

C40. The ophthalmic composition or method of any one of embodiments A1-A33, B1-B147, or C1-C39, wherein the ocular drainage structure identified in the instilled patient's eye around the circumference of the drainage angle is selected from the group consisting of: an aqueous vein, an episcleral vein, an interscleral channel (e.g., collector channel), a collector channel, a collector channel/aqueous/episcleral vein system, an aqueous drainage system, a conjunctival venous system, a subconjunctival space, a trabecular meshwork, a canal of Schlemm, a suprachoroidal space, or a scleral spur.

C41. The ophthalmic composition or method of any one of embodiments A1-A33, B1-B147, or C1-C40, wherein the ocular drainage structure identified in the instilled patient's eye around the circumference of the drainage angle is selected from the group consisting of: an interscleral channel (e.g., collector channel), a collector channel, a collector channel/aqueous/episcleral vein system, an aqueous drainage system, a conjunctival venous system, a subconjunctival space, a trabecular meshwork, a canal of Schlemm, a suprachoroidal space, a scleral spur.

C42. The ophthalmic composition or method of any one of embodiments A1-A33, B1-B147, or C1-C41, wherein the ocular drainage structure identified in the instilled patient's eye around the circumference of the drainage angle is an interscleral channel (e.g., collector channel).

C43. The ophthalmic composition or method of any one of embodiments A1-A33, B1-B147, or C1-C42, wherein the ocular drainage structure identified in the instilled patient's eye around the circumference of the drainage angle a collector channel.

C44. The ophthalmic composition or method of any one of embodiments A1-A33, B1-B147, or C1, wherein the identified ocular drainage structure is a collector channel trunk.

C45. The ophthalmic composition or method of any one of embodiments A1-A33, B1-B147, or C1-C44, wherein the ocular drainage structure identified in the instilled patient's eye around the circumference of the drainage angle is a canal of Schlemm.

C46. The ophthalmic composition or method of any one of embodiments A1-A33, B1-B147, or C1-C45, wherein the ocular drainage structure identified in the instilled patient's eye around the circumference of the drainage angle is a conjunctival venous system.

C47. The ophthalmic composition or method of any one of embodiments A1-A33, B1-B147, or C1-C46, wherein the ocular drainage structure identified in the instilled patient's eye around the circumference of the drainage angle is a subconjunctival space.

C48. The ophthalmic composition or method of any one of embodiments A1-A33, B1-B147, or C1-C47, wherein the ocular drainage structure identified in the instilled patient's eye around the circumference of the drainage angle is a trabecular meshwork.

C49. The ophthalmic composition or method of any one of embodiments A1-A33, B1-B147, or C1-C48, wherein the ocular drainage structure identified in the instilled patient's eye around the circumference of the drainage angle is an aqueous drainage system.

C50. The ophthalmic composition or method of any one of embodiments A1-A33, B1-B147, or C1-C249, wherein the ocular drainage structure identified in the instilled patient's eye around the circumference of the drainage angle is a suprachoroidal space.

C51. The ophthalmic composition or method of any one of embodiments A1-A33, B1-B147, or C1-C50, wherein the identified ocular drainage structure is the deep scleral plexus.

C52. The ophthalmic composition or method of any one of embodiments A1-A33, B1-B147, or C1-CM, wherein the identified ocular drainage structure is the episcleral/aqueous vein(s).

C53. The ophthalmic composition or method of any one of embodiments A1-A33, B1-B147, or C1-C52, wherein the plurality of ophthalmic devices are positioned in, near or adjacent to 2 interscleral channels (e.g., collector channels) identified in the instilled patient's eye around the circumference of the drainage angle.

C54. The ophthalmic composition or method of any one of embodiments A1-A33, B1-B147, or C1-C53, wherein the plurality of ophthalmic devices are positioned in, near or adjacent to 3 interscleral channels (e.g., collector channels) identified in the instilled patient's eye around the circumference of the drainage angle.

C55. The ophthalmic composition or method of any one of embodiments A1-A33, B1-B147, or C1-C54, wherein the plurality of ophthalmic devices are positioned in, near or adjacent to 4 interscleral channels (e.g., collector channels) identified in the instilled patient's eye around the circumference of the drainage angle.

C56. The ophthalmic composition or method of any one of embodiments A1-A33, B1-B147, or C1-C55, wherein the plurality of ophthalmic devices are positioned in, near or adjacent to 5 interscleral channels (e.g., collector channels) identified in the instilled patient's eye around the circumference of the drainage angle.

C57. The ophthalmic composition or method of any one of embodiments A1-A33, B1-B147, or C1-C56, wherein the plurality of ophthalmic devices are positioned in, near or adjacent to 2-6 interscleral channels (e.g., collector channels) identified in the instilled patient's eye around the circumference of the drainage angle.

C58. The ophthalmic composition or method of any one of embodiments A1-A33, B1-B147, or C1-57, wherein the plurality of ophthalmic devices are positioned in, near or adjacent to 3-6 interscleral channels (e.g., collector channels) identified in the instilled patient's eye around the circumference of the drainage angle.

C59. The ophthalmic composition or method of any one of embodiments A1-A33, B1-B147, or C1-C58, wherein the plurality of ophthalmic devices are positioned in, near or adjacent to between 2-20 interscleral channels (e.g., collector channels), 3-20 interscleral channels (e.g., collector channels), 4-20 interscleral channels (e.g., collector channels), 5-20 interscleral channels (e.g., collector channels), 2-10 interscleral channels (e.g., collector channels), 3-10 interscleral channels (e.g., collector channels), 4-10 interscleral channels (e.g., collector channels), 5-10 interscleral channels (e.g., collector channels), 2-5 interscleral channels (e.g., collector channels), or 3-5 interscleral channels (e.g., collector channels), identified in the instilled patient's eye around the circumference of the drainage angle.

C60. The ophthalmic composition or method of any one of embodiments A1-A33, B1-B147, or C1-C59, wherein the method facilitates accurate and/or precise inserting, placement, positioning, repositioning, lifting, and/or removal, of a plurality of ophthalmic devices within the Indigo Carmine instilled patient's eye, wherein the plurality of ophthalmic devices are positioned in a plurality of locations around the circumference of the drainage angle to form a plurality of drain or flow patterns of the aqueous humor in the instilled patient's eye.

C61. The ophthalmic composition or method of any one of embodiments A1-A33, B1-B147, or C1-C60, wherein the plurality of ophthalmic devices introduced into the instilled eye is at least 2 ophthalmic devices.

C62. The ophthalmic composition or method of any one of embodiments A1-A33, B1-B147, or C1-C61, wherein the plurality of ophthalmic devices introduced into the instilled eye is at least 3 ophthalmic devices, at least 4 ophthalmic devices, at least 5 ophthalmic devices, at least 6 ophthalmic devices, at least 7 ophthalmic devices, at least 8 ophthalmic devices, at least 9 ophthalmic devices, at least 10 ophthalmic devices, at least 11 ophthalmic devices, at least 12 ophthalmic devices, at least 13 ophthalmic devices, at least 14 ophthalmic devices, or at least 15 ophthalmic devices.

C63. The ophthalmic composition or method of any one of embodiments A1-A33, B1-B147, or C1-C62, wherein the plurality of ophthalmic devices introduced into the instilled eye is in the range of between 3-20, between 4-20, between 5-20, between 6-20, between 3-10, between 10-20, or between 5-15 ophthalmic devices.

C64. The ophthalmic composition or method of any one of embodiments A1-A33, B1-B147, or C1-C63, wherein the plurality of ophthalmic devices introduced into the instilled eye is in the range of between 2-10.

C65. The ophthalmic composition or method of any one of embodiments A1-A33, B1-B147, or C1-C64, wherein the plurality of ophthalmic devices introduced into the instilled eye is in the range of between 3-10.

C66. The ophthalmic composition or method of any one of embodiments A1-A33, B1-B147, or C1-C65, wherein the plurality of ophthalmic devices introduced into the instilled eye is in the range of between 2-6.

C67. The ophthalmic composition or method of any one of embodiments A1-A33, B1-B147, or C1-C66, wherein the plurality of ophthalmic devices introduced into the instilled eye is in the range of between 3-6.

C68. The ophthalmic composition or method of any one of embodiments A1-A33, B1-B147, or C1-C67, wherein the plurality of ophthalmic devices introduced into the instilled eye is 3, 4, 5, 6, 7, 8, 9, 10, 11, 12, 13, 14, 15, 16, 17, 18, 19, or 20 ophthalmic devices.

C69. The ophthalmic composition or method of any one of embodiments A1-A33, B1-B147, or C1-C68, wherein the plurality of ophthalmic devices are distributed among 2-10 ocular drainage structures identified in the instilled patient's eye around the circumference of the drainage angle, and positioned in, near or adjacent to said identified ocular drainage structures.

C70. The ophthalmic composition or method of any one of embodiments A1-A33, B1-B147, or C1-C69, wherein the plurality of ophthalmic devices are distributed among 3-10 ocular drainage structures identified in the instilled patient's eye around the circumference of the drainage angle, and positioned in, near or adjacent to said identified ocular drainage structures.

C71. The ophthalmic composition or method of any one of embodiments A1-A33, B1-B147, or C1-C70, wherein the plurality of ophthalmic devices are distributed among 2-6 ocular drainage structures identified in the instilled patient's eye around the circumference of the drainage angle, and positioned in, near or adjacent to said identified ocular drainage structures.

C72. The ophthalmic composition or method of any one of embodiments A1-A33, B1-B147, or C1-C71, wherein the plurality of ophthalmic devices are positioned in, near or adjacent to 2, 3, 4, 5, or 6 ocular drainage structures, identified in the instilled patient's eye around the circumference of the drainage angle.

C73. The ophthalmic composition or method of any one of embodiments A1-A33, B1-B147, or C1-C72, wherein the plurality of ophthalmic devices are distributed among 3-6 ocular drainage structures identified in the instilled patient's eye around the circumference of the drainage angle, and positioned in, near or adjacent to said identified ocular drainage structures.

C74. The ophthalmic composition or method of any one of embodiments A1-A33, B1-B147, or C1-C73, wherein the plurality of ophthalmic devices are positioned in, near or adjacent to 3, 4, 5, or 6 ocular drainage structures, identified in the instilled patient's eye around the circumference of the drainage angle.

C75. The ophthalmic composition or method of any one of embodiments A1-A33, B1-B147, or C1-C74, wherein the distribution of the plurality of ophthalmic devices is an equal distribution among the ocular drainage structures identified in the instilled patient's eye around the circumference of the drainage angle.

C76. The ophthalmic composition or method of any one of embodiments A1-A33, B1-B147, or C1-C75, wherein two of the plurality of ophthalmic devices are independently positioned in, near or adjacent to a single identified ocular drainage structure in the instilled patient's eye.

C77. The ophthalmic composition or method of any one of embodiments A1-A33, B1-B147, or C1-C76, wherein the plurality of ophthalmic devices introduced into the instilled eye are positioned around the circumference of the drainage angle in such a manner as to maximize the angle between at least two of the plurality of ophthalmic devices around the circumference of said drainage angle.

C78. The ophthalmic composition or method of any one of embodiments A1-A33, B1-B147, or C1-C77, wherein the plurality of ophthalmic devices introduced into the instilled eye are positioned around the circumference of the drainage angle such that at least 2 of the plurality of ophthalmic devices are separated from each other by 10-45 degrees, 30-45 degrees, 60-120 degrees, 90-270 degrees, 120-240 degrees, 150-210 degrees, 150-180 degrees, or 170-190 degrees, around the circumference of said drainage angle.

C79. The ophthalmic composition or method of any one of embodiments A1-A33, B1-B147, or C1-C78, wherein the plurality of ophthalmic devices introduced into the instilled eye are positioned around the circumference of the drainage angle such that at least 2 of the plurality of ophthalmic devices are separated from each other by 90-270 degrees, 120-240 degrees, 150-210 degrees, or 170-190 degrees, around the circumference of said drainage angle.

C80. The ophthalmic composition or method of any one of embodiments A1-A33, B1-B147, or C1-C79, wherein the plurality of ophthalmic devices introduced into the instilled eye are positioned around the circumference of the drainage angle such that at least 2 of the plurality of ophthalmic devices are separated from each other by 10-30 degrees around the circumference of said drainage angle.

C81. The ophthalmic composition or method of any one of embodiments A1-A33, B1-B147, or C1-C80, wherein the plurality of ophthalmic devices introduced into the instilled eye are positioned around the circumference of the drainage angle such that at least 2 of the plurality of ophthalmic devices are separated from each other by 10-45 degrees around the circumference of said drainage angle.

C82. The ophthalmic composition or method of any one of embodiments A1-A33, B1-B147, or C1-C81, wherein the plurality of ophthalmic devices introduced into the instilled eye are positioned around the circumference of the drainage angle such that at least 2 of the plurality of ophthalmic devices are separated from each other by 30-45 degrees around the circumference of said drainage angle.

C83. The ophthalmic composition or method of any one of embodiments A1-A33, B1-B147, or C1-C82, wherein the plurality of ophthalmic devices introduced into the instilled eye are positioned around the circumference of the drainage angle such that at least 2 of the plurality of ophthalmic devices are separated from each other by 60-120 degrees around the circumference of said drainage angle.

C84. The ophthalmic composition or method of any one of embodiments A1-A33, B1-B147, or C1-C83, wherein the plurality of ophthalmic devices introduced into the instilled eye are positioned around the circumference of the drainage angle such that at least 2 of the plurality of ophthalmic devices are separated from each other by 150-180 degrees around the circumference of said drainage angle.

C85. The ophthalmic composition or method of any one of embodiments A1-A33, B1-B147, or C1-C84, wherein the plurality of ophthalmic devices introduced into the instilled eye are positioned around the circumference of the drainage angle such that at least 3 of the plurality of ophthalmic devices are separated from each other by 10-30 degrees, 30-120 degrees, 30-60 degrees, 40-100 degrees, 50-90 degrees, 60-120 degrees, 80-120 degrees, 90-120 degrees, or 100-120 degrees, around the circumference of said drainage angle.

C86. The ophthalmic composition or method of any one of embodiments A1-A33, B1-B147, or C1-C85, wherein the plurality of ophthalmic devices introduced into the instilled eye are positioned around the circumference of the drainage angle such that at least 3 of the plurality of ophthalmic devices are separated from each other by 30-120 degrees, 30-60 degrees, 40-100 degrees, 50-90 degrees, 60-120 degrees, 80-120 degrees, 90-120 degrees, or 100-120 degrees, around the circumference of said drainage angle.

C87. The ophthalmic composition or method of any one of embodiments A1-A33, B1-B147, or C1-C86, wherein the plurality of ophthalmic devices introduced into the instilled eye are positioned around the circumference of the drainage angle such that at least 3 of the plurality of ophthalmic devices are separated from each other by 10-30 degrees around the circumference of said drainage angle.

C88. The ophthalmic composition or method of any one of embodiments A1-A33, B1-B147, or C1-C87, wherein the plurality of ophthalmic devices introduced into the instilled eye are positioned around the circumference of the drainage angle such that at least 3 of the plurality of ophthalmic devices are separated from each other by 30-60 degrees around the circumference of said drainage angle.

C89. The ophthalmic composition or method of any one of embodiments A1-A33, B1-B147, or C1-C88, wherein the plurality of ophthalmic devices introduced into the instilled eye are positioned around the circumference of the drainage angle such that at least 3 of the plurality of ophthalmic devices are separated from each other by 50-90 degrees around the circumference of said drainage angle.

C90. The ophthalmic composition or method of any one of embodiments A1-A33, B1-B147, or C1-C89, wherein the plurality of ophthalmic devices introduced into the instilled eye are positioned around the circumference of the drainage angle such that at least 3 of the plurality of ophthalmic devices are separated from each other by 40-120 degrees around the circumference of said drainage angle.

C91. The ophthalmic composition or method of any one of embodiments A1-A33, B1-B147, or C1-C90, wherein the plurality of ophthalmic devices introduced into the instilled eye are positioned around the circumference of the drainage angle such that at least 3 of the plurality of ophthalmic devices are separated from each other by 60-120 degrees around the circumference of said drainage angle.

C92. The ophthalmic composition or method of any one of embodiments A1-A33, B1-B147, or C1-C91, wherein the plurality of ophthalmic devices introduced into the instilled eye are positioned around the circumference of the drainage angle such that at least 3 of the plurality of ophthalmic devices are separated from each other by 90-120 degrees around the circumference of said drainage angle.

C93. The ophthalmic composition or method of any one of embodiments A1-A33, B1-B147, or C1-C92, wherein the plurality of ophthalmic devices introduced into the instilled eye are positioned around the circumference of the drainage angle such that at least 4 of the plurality of ophthalmic devices are separated from each other by 10-30 degrees, 30-50 degrees, 30-80 degrees, 30-90 degrees, 50-70 degrees, or 60-90 degrees, around the circumference of said drainage angle.

C94. The ophthalmic composition or method of any one of embodiments A1-A33, B1-B147, or C1-C93, wherein the plurality of ophthalmic devices introduced into the instilled eye are positioned around the circumference of the drainage angle such that at least 4 of the plurality of ophthalmic devices are separated from each other by 30-50 degrees, 30-80 degrees, 30-90 degrees, 50-70 degrees, or 60-90 degrees, around the circumference of said drainage angle.

C95. The ophthalmic composition or method of any one of embodiments A1-A33, B1-B147, or C1-C94, wherein the plurality of ophthalmic devices introduced into the instilled eye are positioned around the circumference of the drainage angle such that at least 4 of the plurality of ophthalmic devices are separated from each other by 10-30 degrees around the circumference of said drainage angle.

C96. The ophthalmic composition or method of any one of embodiments A1-A33, B1-B147, or C1-C95, wherein the plurality of ophthalmic devices introduced into the instilled eye are positioned around the circumference of the drainage angle such that at least 4 of the plurality of ophthalmic devices are separated from each other by 30-50 degrees around the circumference of said drainage angle.

C97. The ophthalmic composition or method of any one of embodiments A1-A33, B1-B147, or C1-C96, wherein the plurality of ophthalmic devices introduced into the instilled eye are positioned around the circumference of the drainage angle such that at least 4 of the plurality of ophthalmic devices are separated from each other by 30-90 degrees around the circumference of said drainage angle.

C98. The ophthalmic composition or method of any one of embodiments A1-A33, B1-B147, or C1-C97, wherein the plurality of ophthalmic devices introduced into the instilled eye are positioned around the circumference of the drainage angle such that at least 4 of the plurality of ophthalmic devices are separated from each other by 60-90 degrees around the circumference of said drainage angle.

C99. The ophthalmic composition or method of any one of embodiments A1-A33, B1-B147, or C1-C98, wherein the plurality of ophthalmic devices introduced into the instilled eye are positioned around the circumference of the drainage angle to maintain physiological aqueous humor circulation of the patient's eye following the ocular surgery.

C100. The ophthalmic composition or method of any one of embodiments A1-A33, B1-B147, or C1-C99, wherein the plurality of ophthalmic devices introduced into the instilled eye are positioned around the circumference of the drainage angle to maintain corneal nutrition of the patient's eye following the ocular surgery.

C101. The ophthalmic composition or method of any one of embodiments A1-A33, B1-B147, or C1-C100, wherein the plurality of ophthalmic devices introduced into the instilled eye are positioned around the circumference of the drainage angle to maintain adequate corneal nutrition of the patient's eye following the ocular surgery.

C102. The ophthalmic composition or method of any one of embodiments A1-A33, B1-B147, or C1-C101, wherein the plurality of ophthalmic devices introduced into the instilled eye are positioned around the circumference of the drainage angle to maintain uniform corneal nutrition of the patient's eye following the ocular surgery.

C103. The ophthalmic composition or method of any one of embodiments A1-A33, B1-B147, or C1-C102, wherein the plurality of ophthalmic devices introduced into the instilled eye are positioned around the circumference of the drainage angle to maintain corneal endothelial cell density of the patient's eye following the ocular surgery.

C104. The ophthalmic composition or method of any one of embodiments A1-A33, B1-B147, or C1-C103, wherein the plurality of ophthalmic devices introduced into the instilled eye are positioned around the circumference of the drainage angle to mitigate corneal endothelial cell loss of the patient's eye following the ocular surgery.

C105. The ophthalmic composition or method of any one of embodiments A1-A33, B1-B147, or C1-C104, wherein the plurality of ophthalmic devices introduced into the instilled eye are positioned around the circumference of the drainage angle to form a plurality of circulation patterns of the aqueous humor within the patient's eye following the ocular surgery.

C106. The ophthalmic composition or method of any one of embodiments A1-A33, B1-B147, or C1-C105, wherein the plurality of ophthalmic devices introduced into the instilled eye are positioned around the circumference of the drainage angle to form a plurality of drain flow patterns of the aqueous humor within the patient's eye following the ocular surgery.

C107. The ophthalmic composition or method of any one of embodiments A1-A33, B1-B147, or C1-C106, wherein the plurality of ophthalmic devices introduced into the instilled eye are positioned around the circumference of the drainage angle; and wherein the ocular surgery is selected from the group consisting of: glaucoma surgery, minimally invasive glaucoma surgery (MIGS), cataract surgery, retinal surgery, lens replacement surgery, surgery to treat ocular trauma, refractive lensectomy, corneal surgery, endothelial keratoplasty, Descemet's Membrane Endothelial Keratoplasty (DMEK), capsulorhexis, lamellar corneal transplantation, minimally invasive corneal procedure, corneal refractive procedure, small incision lenticule extraction (SMILE), Ab interno Canaloplasty (ABiC), Ab externo Canaloplasty (ABeC), retinal procedures such as removal of epiretinal membranes, and ocular surface diagnostic technique.

C108. The ophthalmic composition or method of any one of embodiments A1-A33, B1-B147, or C1-C107, wherein the plurality of ophthalmic devices introduced into the instilled eye are positioned around the circumference of the drainage angle; and wherein the ocular surgery is glaucoma surgery.

C109. The ophthalmic composition or method of any one of embodiments A1-A33, B1-B147, or C1-C108, wherein the plurality of ophthalmic devices introduced into the instilled eye are positioned around the circumference of the drainage angle; and wherein the ocular surgery is minimally invasive glaucoma surgery (MIGS).

C110. The ophthalmic composition or method of any one of embodiments A1-A33, B1-B147, or C1-C109, wherein the plurality of ophthalmic devices introduced into the instilled eye are positioned around the circumference of the drainage angle; and wherein the ocular surgery is cataract surgery.

C111. The ophthalmic composition or method of any one of embodiments A1-A33, B1-B147, or C1-C110, wherein the plurality of positions or plurality of locations the plurality of ophthalmic devices are introduced into the instilled eye around the circumference of the drainage angle are selected from the group consisting of: a fine vessel, an aqueous vein, an episcleral vein, a collector channel (interscleral channel), a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

C112. The ophthalmic composition or method of any one of embodiments A1-A33, B1-B147, or C1-C111, wherein the ocular drainage structures identified in the instilled eye around the circumference of the drainage angle are selected from the group consisting of: a fine vessel, an aqueous vein, an episcleral vein, a collector channel (interscleral channel), a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

C113. The ophthalmic composition or method of any one of embodiments A1-A33, B1-B147, or C1-C112, wherein the ocular drainage structures identified in the instilled eye around the circumference of the drainage angle are collector channels.

C114. The ophthalmic composition or method of any one of embodiments A1-A33, B1-B147, or C1-C113, wherein one or more stents of the plurality of stents is placed in, proximate, or adjacent to, an identified collector channel.

C115. The ophthalmic composition or method of any one of embodiments A1-A33, B1-B147, or C1-C114, wherein one or more stents of the plurality of stents is placed in, proximate, or adjacent to, an identified collector channel trunk.

C116. The ophthalmic composition or method of any one of embodiments A1-A33, B1-B147, or C1-C115, wherein the ocular drainage structure identified in the instilled eye around the circumference of the drainage angle is the canal of Schlemm.

C117. The ophthalmic composition or method of any one of embodiments A1-A33, B1-B147, or C1-C116, wherein one or more stents of the plurality of stents is placed in, proximate, or adjacent to, an identified canal of Schlemm.

C118. The ophthalmic composition or method of any one of embodiments A1-A33, B1-B147, or C1-C117, wherein the ocular drainage structures identified in the instilled eye around the circumference of the drainage angle is an aqueous drainage system.

C119. The ophthalmic composition or method of any one of embodiments A1-A33, B1-B147, or C1-C118, wherein the ocular drainage structures identified in the instilled eye around the circumference of the drainage angle is a conjunctival venous system.

C120. The ophthalmic composition or method of any one of embodiments A1-A33, B1-B147, or C1-C119, wherein the ocular drainage structures identified in the instilled eye around the circumference of the drainage angle is a subconjunctival space.

C121. The ophthalmic composition or method of any one of embodiments A1-A33, B1-B147, or C1-C120, wherein one or more stents of the plurality of stents is placed in, proximate, or adjacent to, a subconjunctival space.

C122. The ophthalmic composition or method of any one of embodiments A1-A33, B1-B147, or C1-C121, wherein one or more stents of the plurality of stents is placed in, proximate, or adjacent to, an identified deep scleral plexus.

C123. The ophthalmic composition or method of any one of embodiments A1-A33, B1-B147, or C1-C122, wherein one or more stents of the plurality of stents is placed in, proximate, or adjacent to, an identified episcleral/aqueous vein.

C124. The ophthalmic composition or method of any one of embodiments A1-A33, B1-B147, or C1-C123, wherein the ocular drainage structures identified in the instilled eye around the circumference of the drainage angle is a trabecular meshwork.

C125. The ophthalmic composition or method of any one of embodiments A1-A33, B1-B147, or C1-C124, wherein one or more stents of the plurality of stents is placed in, proximate, or adjacent to, an identified trabecular meshwork.

C126. The ophthalmic composition or method of any one of embodiments A1-A33, B1-B147, or C1-C125, wherein the ocular drainage structures identified in the instilled eye around the circumference of the drainage angle is a suprachoroidal space.

C127. The ophthalmic composition or method of any one of embodiments A1-A33, B1-B147, or C1-C126, wherein one or more stents of the plurality of stents is placed in, proximate, or adjacent to, an identified suprachoroidal space.

C128. The ophthalmic composition or method of any one of embodiments A1-A33, B1-B147, or C1-C127, wherein at least one of the plurality of ophthalmic devices introduced into the instilled eye that is positioned around the circumference of the drainage angle to form a plurality of drain flow patterns of the aqueous humor within the patient's eye following the ocular surgery is located approximately opposite a main collector channel.

C129. The ophthalmic composition or method of any one of embodiments A1-A33, B1-B147, or C1-C128, wherein at least one of the plurality of ophthalmic devices introduced into the instilled eye that is positioned around the circumference of the drainage angle to form a plurality of drain flow patterns of the aqueous humor within the patient's eye following the ocular surgery is located approximately opposite a main collector channel identified by the instilled composition.

C130. The ophthalmic composition or method of any one of embodiments A1-A33, B1-B147, or C1-C129, wherein at least one of the plurality of ophthalmic devices introduced into the instilled eye is positioned around the circumference of the drainage angle and in, near or adjacent to an ocular drainage structure located approximately opposite a main collector channel identified by the instilled composition.

C131. The ophthalmic composition or method of any one of embodiments A1-A33, B1-B147, or C1-C130, wherein at least two of the plurality of ophthalmic devices introduced into the instilled eye are positioned around the circumference of the drainage angle and in, near or adjacent to two ocular drainage structures located approximately opposite a main collector channel identified by the instilled composition.

C132. The ophthalmic composition or method of any one of embodiments A1-A33, B1-B147, or C1-C131, wherein at least three of the plurality of ophthalmic devices introduced into the instilled eye are positioned around the circumference of the drainage angle and in, near or adjacent to three ocular drainage structures located approximately opposite a main collector channel identified by the instilled composition.

C133. The ophthalmic composition or method of any one of embodiments A1-A33, B1-B147, or C1-C132, wherein a plurality or each of the plurality of ophthalmic devices has an internal diameter of between 5-500 microns, 5-450 microns, 5-420 microns, 5-400 microns, 5-375 microns, 5-350 microns, 5-325 microns, 5-300 microns, 5-275 microns, 5-205 microns, 5-250 microns, 5-225 microns, 5-200 microns, 5-175 microns, 5-150 microns, 5-125 microns, 5-100 microns, or 5-50 microns, 50-500 microns, 50-400 microns, 50-300 microns, 50-200 microns, 50-100 microns, 100-500 microns, 100-400 microns, 100-300 microns, 100-200 microns, 200-500 microns, 200-400 microns, 200-300 microns, 300-500 microns, 300-400 microns, or 400-500 microns, plus or minus 4 microns.

C134. The ophthalmic composition or method of any one of embodiments A1-A33, B1-B147, or C1-C133, wherein the plurality of ophthalmic devices is 2-10 ophthalmic devices, wherein each ophthalmic device independently has an internal diameter of about 50-80 microns, plus or minus 4 microns.

C135. The ophthalmic composition or method of any one of embodiments A1-A33, B1-B147, or C1-C134, wherein the plurality of ophthalmic devices is 3-10 ophthalmic devices, wherein each ophthalmic device independently has an internal diameter of about 50-80 microns, plus or minus 4 microns.

C136. The ophthalmic composition or method of any one of embodiments A1-A33, B1-B147, or C1-C135, wherein the plurality of ophthalmic devices is 2-6 ophthalmic devices, wherein each ophthalmic device independently has an internal diameter of about 50-80 microns, plus or minus 4 microns.

C137. The ophthalmic composition or method of any one of embodiments A1-A33, B1-B147, or C1-C136, wherein the plurality of ophthalmic devices is 3-6 ophthalmic devices, wherein each ophthalmic device independently has an internal diameter of about 50-80 microns, plus or minus 4 microns.

C138. The ophthalmic composition or method of any one of embodiments A1-A33, B1-B147, or C1-C137, wherein the plurality of ophthalmic devices introduced into the instilled eye have a cumulative internal diameter of at least 50 microns and no more than 500 microns, plus or minus 4 microns.

C139. The ophthalmic composition or method of any one of embodiments A1-A33, B1-B147, or C1-C138, wherein a plurality or each of the plurality of ophthalmic devices has an internal diameter of about 5, 10, 15, 20, 30, 40, 50, 60, 70, 80, 90, 100, 125, 150, 175, 200, 225, 250, 275, 300, 325, 350, 375, 400, 425, 450, 475, or 500 microns, plus or minus 4 microns.

C140. The ophthalmic composition or method of any one of embodiments A1-A33, B1-B147, or C1-C139, wherein a plurality or each of the plurality of ophthalmic devices has a length of between 5-20 mm, 5-19 mm, 5-18 mm, 5-17 mm, 5-16 mm, 5-15 mm, 5-10 mm, 10-20 mm, 10-19 mm, 10-18 mm, 10-17 mm, 10-16 mm, or 10-15 mm, plus or minus 0.5 mm.

C141. The ophthalmic composition or method of any one of embodiments A1-A33, B1-B147, or C1-C140, wherein a plurality or each of the plurality of ophthalmic devices has a length of about 5, 6, 7, 8, 9, 10, 11, 12, 13, 14, 15, 16, 17, 18, 19, or 20 mm, plus or minus 0.5 mm.

C142. The ophthalmic composition or method of any one of embodiments A1-A33, B1-B147, or C1-C141, wherein a plurality or each of the plurality of ophthalmic devices is composed of a material, or coated with a material, or contains an inner layer of a material, selected from the group consisting of: poly(styrene-block-isobutylene-block-styrene (SIBS), Polyimide, heparin-coated non-ferromagnetic titanium, nitinol, collagen-derived porcine gelatine cross-linked with glutaraldehyde, or derivatives thereof.

C143. The ophthalmic composition or method of any one of embodiments A1-A33, B1-B147, or C1-C142, wherein a plurality or each of the plurality of ophthalmic devices is composed of a material, or coated with a material, or contains an inner layer of a material, of poly(styrene-block-isobutylene-block-styrene (SIBS), or derivative thereof.

C144. The ophthalmic composition or method of any one of embodiments A1-A33, B1-B147, or C1-C143, wherein the plurality of ophthalmic devices introduced into the instilled eye are positioned around the circumference of the drainage angle to maintain an average IOP level of at least 10 mmHg.

C145. The ophthalmic composition or method of any one of embodiments A1-A33, B1-B147, or C1-C144, wherein the plurality of ophthalmic devices introduced into the instilled eye have a cumulative internal diameter such that the total flow resistance of the plurality of ophthalmic devices maintains an IOP level in the treated patient's eye of at least 10 mm Hg.

C146. The ophthalmic composition or method of any one of embodiments A1-A33, B1-B147, or C1-C145, wherein the positioning of the plurality of ophthalmic devices in, near or adjacent to identified ocular drainage structures located around the circumference of the drainage angle in the instilled patient's eye maintains an average IOP of 10 mmHg of the treated patient's eye.

C147. The ophthalmic composition or method of any one of embodiments A1-A33, B1-B147, or C1-C146, wherein internal diameters of the plurality of ophthalmic devices have sufficient flow resistance such that the resulting IOP of the treated patient's eye maintains a level of at least 10 mm Hg.

C148. The ophthalmic composition or method of any one of embodiments A1-A33, B1-B147, or C1-C147, wherein each of the plurality of ophthalmic devices has an internal diameter with sufficient flow resistance such that the resulting IOP of the treated patient's eye maintains a level of at least 10 mm Hg.

C149. The ophthalmic composition or method of any one of embodiments A1-A33, B1-B147, or C1-C148, wherein the plurality of ophthalmic devices introduced into the instilled eye are positioned around the circumference of the drainage angle to maintain an average IOP level of at least 10 mmHg and less than 15 mmHg.

C150. The ophthalmic composition or method of any one of embodiments A1-A33, B1-B147, or C1-C149, wherein the plurality of ophthalmic devices introduced into the instilled eye have a cumulative internal diameter such that the total flow resistance of the plurality of ophthalmic devices maintains an IOP level in the treated patient's eye of at least 10 mm Hg and no more than 15 mmHg.

C151. The ophthalmic composition or method of any one of embodiments A1-A33, B1-B147, or C1-C150, wherein each of the plurality of ophthalmic devices has an internal diameter with sufficient flow resistance such that the resulting IOP of the treated patient's eye maintains a level of at least 10 mm Hg and no more than 15 mm Hg.

C152. The ophthalmic composition or method of any one of embodiments A1-A33, B1-B147, or C1-C151, wherein the positioning of the plurality of ophthalmic devices in, near or adjacent to identified ocular drainage structures located around the circumference of the drainage angle in the instilled patient's eye maintains an IOP of at least 10 mmHg and less than 15 mmHg of the treated patient's eye.

C153. The ophthalmic composition or method of any one of embodiments A1-A33, B1-B147, or C1-C152, wherein the patient's IOP levels following introduction of the plurality of ophthalmic devices maintains an average IOP level of at least 10 mm Hg and below 21 mm Hg.

C154. The ophthalmic composition or method of any one of embodiments A1-A33, B1-B147, or C1-C153, wherein the patient's average IOP levels following introduction of the plurality of ophthalmic devices are at least 10 mm Hg and no more than 15 mm Hg.

C155. The ophthalmic composition or method of any one of embodiments A1-A33, B1-B147, or C1-C154, wherein the patient's average IOP levels following introduction of the plurality of ophthalmic devices are at least 10 mm Hg and no more than 14 mm Hg.

C156. The ophthalmic composition or method of any one of embodiments A1-A33, B1-B147, or C1-C155, wherein the patient's average IOP levels following introduction of the plurality of ophthalmic devices are at least 10 mm Hg and no more than 13 mm Hg.

C157. The ophthalmic composition or method of any one of embodiments A1-A33, B1-B147, or C1-C156, wherein the patient's IOP levels following introduction of the plurality of ophthalmic devices maintains and/or achieves an average IOP level, relative to a human of similar age having a healthy average IOP level.

C158. The ophthalmic composition or method of any one of embodiments A1-A33, B1-B147, or C1-C157, wherein the patient's density of corneal endothelium cells following introduction of the plurality of ophthalmic devices maintains and/or achieves an average of between 3000-4000 cells per square mm.

C159. The ophthalmic composition or method of any one of embodiments A1-A33, B1-B147, or C1-C158, wherein the patient's density of corneal endothelium cells located centrally of the cornea following introduction of the plurality of ophthalmic devices maintains and/or achieves an average of between 3000-4000 cells per square mm.

C160. The ophthalmic composition or method of any one of embodiments A1-A33, B1-B147, or C1-C159, wherein the patient's density of corneal endothelium cells located on the periphery of the cornea following introduction of the plurality of ophthalmic devices maintains and/or achieves an average of between 3000-4000 cells per square mm.

C161. The ophthalmic composition or method of any one of embodiments A1-A33, B1-B147, or C1-C160, wherein the patient's density of corneal endothelium cells located centrally and located on the periphery of the cornea following introduction of plurality of ophthalmic devices maintains and/or achieves an average of between 3000-4000 cells per square mm.

C162. The ophthalmic composition or method of any one of embodiments A1-A33, B1-B147, or C1-C161, wherein utilization of the method provided herein approximates or replicates the physiological state of that particular eye (e.g., physiological aqueous humor circulation, physiological corneal endothelial health, and physiological corneal health)—providing a type of custom drainage procedure approximating physiological conditions.

C163. The ophthalmic composition or method of any one of embodiments A1-A33, B1-B147, or C1-C162, wherein the physiological aqueous humor circulation, corneal endothelial health, and corneal health, of said eye would be maintained by a plurality of stent placements positioned and distributed around the circumference of the drainage angle of the patient's eye.

C164. The ophthalmic composition or method of any one of embodiments A1-A33, B1-B147, or C1-C163, wherein the patient's density of corneal endothelium cells following introduction of the plurality of ophthalmic devices maintains and/or achieves a density level, relative to a human of similar age having a healthy corneal endothelium layer.

C165. The ophthalmic composition or method of any one of embodiments A1-A33, B1-B147, or C1-C164, wherein the aqueous humor circulation of the patient's eye following introduction of the plurality of ophthalmic devices maintains and/or achieves a level of a healthy human of similar age having a healthy aqueous humor circulation, such as determined by fluorophtomtetry.

C166. The ophthalmic composition or method of any one of embodiments A1-A33, B1-B147, or C1-C165, wherein the cell circulation in the anterior chamber of the patient's eye following introduction of the plurality of ophthalmic devices maintains and/or achieves a level of a healthy human of similar age having a healthy cell circulation in the anterior chamber, such as determined by optical coherence.

C167. The ophthalmic composition or method of any one of embodiments A1-A33, B1-B147, or C1-C166, wherein the thickness of the corneal endothelium of the patient's eye following introduction of the plurality of ophthalmic devices maintains and/or achieves a level of thickness in a human of similar age having a healthy corneal endothelium layer.

C168. The ophthalmic composition or method of any one of embodiments A1-A33, B1-B147, or C1-C167, wherein the health of the corneal endothelium of the patient's eye following introduction of the plurality of ophthalmic devices maintains and/or achieves a level of a healthy human of similar age having a healthy corneal endothelium layer.

C169. The ophthalmic composition or method of any one of embodiments A1-A33, B1-B147, or C1-C168, wherein the health of the aqueous humor circulation of the patient's eye following introduction of the plurality of ophthalmic devices maintains and/or achieves a level of a healthy human of similar age having a healthy aqueous humor circulation.

C170. The ophthalmic composition or method of any one of embodiments A1-A33, B1-B147, or C1-C169, wherein utilization of the method provided herein approximates or replicates physiological aqueous humor circulation.

C171. The ophthalmic composition or method of any one of embodiments A1-A33, B1-B147, or C1-C170, wherein the method maintains or approximates physiological aqueous humor circulation.

C172. The ophthalmic composition or method of any one of embodiments A1-A33, B1-B147, or C1-C171, wherein utilization of the method provided herein approximates or replicates physiological corneal endothelial health.

C173. The ophthalmic composition or method of any one of embodiments A1-A33, B1-B147, or C1-C172, wherein the method maintains or approximates physiological corneal endothelial health.

C174. The ophthalmic composition or method of any one of embodiments A1-A33, B1-B147, or C1-C173, wherein utilization of the method provided herein approximates or replicates physiological corneal health.

C175. The ophthalmic composition or method of any one of embodiments A1-A33, B1-B147, or C1-C174, wherein the method maintains or approximates physiological corneal nutrition.

C176. The ophthalmic composition or method of any one of embodiments A1-A33, B1-B147, or C1-C175, wherein the method maintains or approximates physiological corneal endothelial cell density and/or mitigates corneal endothelial cell loss.

C177. The ophthalmic composition or method of any one of embodiments A1-A33, B1-B147, or C1-C176, wherein instillation of Indigo Carmine into the patient's eye facilitates identification of ocular drainage structures, such as major outflow pathways, in the instilled eye.

C178. The ophthalmic composition or method of any one of embodiments A1-A33, B1-B147, or C1-C177, wherein the instillation of Indigo Carmine is in combination with another ophthalmic dye.

C179. The ophthalmic composition or method of any one of embodiments A1-A33, B1-B147, or C1-C178, wherein the Indigo Carmine instillation and the another ophthalmic dye instillation are performed concurrently, sequentially, or instilled into the eye as single composition mixture.

C180. The ophthalmic composition or method of any one of embodiments A1-A33, B1-B147, or C1-C179, wherein the another ophthalmic dye is Trypan Blue.

C181. The ophthalmic composition or method of any one of embodiments A1-A33, B1-B147, or C1-C180, wherein placement of the first ophthalmic device into the patient's eye is after instillation with the ophthalmic composition comprising Indigo Carmine.

C182. The ophthalmic composition or method of any one of embodiments A1-A33, B1-B147, or C1-C181, wherein placement of a first ophthalmic device is positioned in, near or adjacent to an Indigo Carmine identified ocular drainage structure in the instilled patient's eye around the circumference of the drainage angle.

C183. The ophthalmic composition or method of any one of embodiments A1-A33, B1-B147, or C1-C182, wherein the methods facilitates placement of an ophthalmic device without the need of guessing or estimating the position of an ocular drainage structure.

C184. The ophthalmic composition or method of any one of embodiments A1-A33, B1-B147, or C1-C183, wherein the methods facilitates placement of the plurality of ophthalmic devices without the need of guessing or estimating the position of an ocular drainage structure.

C185. The ophthalmic composition or method of any one of embodiments A1-A33, B1-B147, or C1-C184, wherein the ophthalmic device is a stent.

C186. The ophthalmic composition or method of any one of embodiments A1-A33, B1-B147, or C1-C185, wherein the ophthalmic device is a glaucoma stent.

C187. The ophthalmic composition or method of any one of embodiments A1-A33, B1-B147, or C1-C186, wherein at least 1 or at least 2 stents of the plurality of stents are placed in, proximate, or adjacent to, 1 or 2 identified ocular drainage structures located approximately opposite a main collector channel.

C188. The ophthalmic composition or method of any one of embodiments A1-A33, B1-B147, or C1-C187, wherein a plurality of stents are placed in, proximate, or adjacent to, an equal plurality of the identified ocular drainage structures.

C189. The ophthalmic composition or method of any one of embodiments A1-A33, B1-B147, or C1-C188, wherein 2, 3, 4, 5, or 6 stents are placed in, proximate, or adjacent to, 2, 3, 4, 5, or 6 of the identified ocular drainage structures, respectively.

C190. The ophthalmic composition or method of any one of embodiments A1-A33, B1-B147, or C1-C189, wherein 3, 4, 5, or 6 stents are placed in, proximate, or adjacent to, 3, 4, 5, or 6 of the identified ocular drainage structures, respectively.

C191. The ophthalmic composition or method of any one of embodiments A1-A33, B1-B147, or C1-C190, wherein 2 stents are placed in, proximate, or adjacent to, 2 of the identified ocular drainage structures.

C192. The ophthalmic composition or method of any one of embodiments A1-A33, B1-B147, or C1-C191, wherein 3 stents are placed in, proximate, or adjacent to, 3 of the identified ocular drainage structures.

C193. The ophthalmic composition or method of any one of embodiments A1-A33, B1-B147, or C1-C192, wherein 4 stents are placed in, proximate, or adjacent to, 4 of the identified ocular drainage structures.

C194. The ophthalmic composition or method of any one of embodiments A1-A33, B1-B147, or C1-C193, wherein 5 stents are placed in, proximate, or adjacent to, 5 of the identified ocular drainage structures.

C195. The ophthalmic composition or method of any one of embodiments A1-A33, B1-B147, or C1-C194, wherein 6 stents are placed in, proximate, or adjacent to, 6 of the identified ocular drainage structures.

C196. The ophthalmic composition or method of any one of embodiments A1-A33, B1-B147, or C1-C195, wherein 2 stents are placed in, proximate, or adjacent to, a single identified ocular drainage structure.

C197. The ophthalmic composition or method of any one of embodiments A1-A33, B1-B147, or C1-C196, wherein 2 stents of the plurality of stents are placed in, proximate, or adjacent to, a single identified ocular drainage structure, and the remaining of the plurality of stents are distributed among an equal remaining plurality number of the identified ocular drainage structures.

C198. The ophthalmic composition or method of any one of embodiments A1-A33, B1-B147, or C1-C197, wherein 2 stents of the plurality of stents are placed in, proximate, or adjacent to, a single identified ocular drainage structure, and the remaining 1, 2, 3, 4, or 5 stents are distributed among an equal remaining plurality number of the identified ocular drainage structures.

C199. The ophthalmic composition or method of any one of embodiments A1-A33, B1-B147, or C1-C198, wherein 2 stents of the plurality of stents are placed in, proximate, or adjacent to, a single identified ocular drainage structure, and the remaining 1, 2, 3, or 4 stents are distributed among an equal remaining plurality number of the identified ocular drainage structures.

C200. The ophthalmic composition or method of any one of embodiments A1-A33, B1-B147, or C1-C199, wherein 3 stents are placed in, proximate, or adjacent to, a single identified ocular drainage structure.

C201. The ophthalmic composition or method of any one of embodiments A1-A33, B1-B147, or C1-C200, wherein 3 stents are placed in, proximate, or adjacent to, a single identified ocular drainage structure, and the remaining stents of the plurality of stents are placed in, proximate, or adjacent to, an equal remaining plurality number of the identified ocular drainage structures.

C202. The ophthalmic composition or method of any one of embodiments A1-A33, B1-B147, or C1-C201, wherein 3 stents of the plurality of stents are placed in, proximate, or adjacent to, a single identified ocular drainage structure, and the remaining 1, 2, 3, or 4 stents are distributed among an equal remaining plurality number of the identified ocular drainage structures.

C203. The ophthalmic composition or method of any one of embodiments A1-A33, B1-B147, or C1-C202, wherein 3 stents of the plurality of stents are placed in, proximate, or adjacent to, a single identified ocular drainage structure, and the remaining 1, 2, or 3 stents are distributed among an equal remaining plurality number of the identified ocular drainage structures.

C204. The ophthalmic composition or method of any one of embodiments A1-A33, B1-B147, or C1-C203, wherein 6 stents are placed in, proximate, or adjacent to, identified ocular drainage structures, such that the 6 stents are distributed among 5, 4, 3 or 2 identified ocular drainage structures.

C205. The ophthalmic composition or method of any one of embodiments A1-A33, B1-B147, or C1-C204, wherein 6 stents are placed in, proximate, or adjacent to, identified ocular drainage structures, such that the 6 stents are distributed among 4, 3, or 2, identified ocular drainage structures.

C206. The ophthalmic composition or method of any one of embodiments A1-A33, B1-B147, or C1-C205, wherein 5 stents are placed in, proximate, or adjacent to, identified ocular drainage structures, such that the 5 stents are distributed among 4, 3, or 2 identified ocular drainage structures.

C207. The ophthalmic composition or method of any one of embodiments A1-A33, B1-B147, or C1-C206, wherein 4 stents are placed in, proximate, or adjacent to, identified ocular drainage structures, such that the 4 stents are distributed among 3 or 2 identified ocular drainage structures.

C208. The ophthalmic composition or method of any one of embodiments A1-A33, B1-B147, or C1-C207, wherein the plurality of ophthalmic devices is a plurality of stents.

C209. The ophthalmic composition or method of any one of embodiments A1-A33, B1-B147, or C1-C208, wherein each of the plurality of ophthalmic devices is a stent.

C210. The ophthalmic composition or method of any one of embodiments A1-A33, B1-B147, or C1-C209, wherein the ophthalmic device is a glaucoma stent or suprachoroidal stent.

C211. The ophthalmic composition or method of any one of embodiments A1-A33, B1-B147, or C1-C210, wherein the plurality of ophthalmic devices are independently selected from the group consisting of: a glaucoma stent, a suprachoroidal stent, iStent (Glaukos), Hydrus microstent (Ivantis), CyPass (Alcon), iStent Supra (Glaukos), Solx gold shunt (Solx), XEN Gel Stent (Allergan), and/or InnFocus MicroShunt (InnFocus, Santen).

C212. The ophthalmic composition or method of any one of embodiments A1-A33, B1-B147, or C1, -C211, wherein the plurality of ophthalmic devices are glaucoma stents.

C213. The ophthalmic composition or method of any one of embodiments A1-A33, B1-B147, or C1-C212, wherein the plurality of ophthalmic devices are iStent (Glaukos).

C214. The ophthalmic composition or method of any one of embodiments A1-A33, B1-B147, or C1-C213, wherein the plurality of ophthalmic devices are CyPass (Alcon).

C215. The ophthalmic composition or method of any one of embodiments A1-A33, B1-B147, or C1-C214, wherein the plurality of ophthalmic devices are iStent Supra (Glaukos).

C216. The ophthalmic composition or method of any one of embodiments A1-A33, B1-B147, or C1-C215, wherein the plurality of ophthalmic devices are XEN Gel Stent (Allergan).

C217. The ophthalmic composition or method of any one of embodiments A1-A33, B1-B147, or C1-C216, wherein the plurality of ophthalmic devices are suprachoroidal stents.

C218. The ophthalmic composition or method of any one of embodiments A1-A33, B1-B147, or C1-C217, wherein the plurality of ophthalmic devices comprises a glaucoma stent and/or a suprachoroidal stent.

C219. The ophthalmic composition or method of any one of embodiments A1-A33, B1-B147, or C1-C218, wherein the placement of the ophthalmic device near, proximate, or adjacent to, the identified ocular structure is within a distance of 1-5 inner diameters of said ophthalmic device, and wherein the ophthalmic device is a stent.

C220. The ophthalmic composition or method of any one of embodiments A1-A33, B1-B147, or C1-C219, wherein the placement of the ophthalmic device near, proximate, or adjacent to, the identified ocular structure is within a distance of 1-3 inner diameters of said ophthalmic device, and wherein the ophthalmic device is a stent.

C221. The ophthalmic composition or method of any one of embodiments A1-A33, B1-B147, or C1-C220, wherein the placement of the ophthalmic device near, proximate, or adjacent to, the identified ocular structure is within a distance of 1-2 inner diameters of said ophthalmic device, and wherein the ophthalmic device is a stent.

C222. The ophthalmic composition or method of any one of embodiments A1-A33, B1-B147, or C1-C221, wherein the placement of the ophthalmic device near, proximate, or adjacent to, the identified ocular structure is within a distance of 5 inner diameters of said ophthalmic device, and wherein the ophthalmic device is a stent.

C223. The ophthalmic composition or method of any one of embodiments A1-A33, B1-B147, or C1-C222, wherein the placement of the ophthalmic device near, proximate, or adjacent to, the identified ocular structure is within a distance of 4 inner diameters of said ophthalmic device, and wherein the ophthalmic device is a stent.

C224. The ophthalmic composition or method of any one of embodiments A1-A33, B1-B147, or C1-C223, wherein the placement of the ophthalmic device near, proximate, or adjacent to, the identified ocular structure is within a distance of 3 inner diameters of said ophthalmic device, and wherein the ophthalmic device is a stent.

C225. The ophthalmic composition or method of any one of embodiments A1-A33, B1-B147, or C1-C224, wherein the placement of the ophthalmic device near, proximate, or adjacent to, the identified ocular structure is within a distance of 2 inner diameters of said ophthalmic device, and wherein the ophthalmic device is a stent.

C226. The ophthalmic composition or method of any one of embodiments A1-A33, B1-B147, or C1-C225, wherein the placement of the ophthalmic device near, proximate, or adjacent to, the identified ocular structure is within a distance of 1 inner diameter of said ophthalmic device, and wherein the ophthalmic device is a stent.

C227. The ophthalmic composition or method of any one of embodiments A1-A33, B1-B147, or C1-C226, wherein the ophthalmic composition disclosed herein has a reduced amount of oxygen content, relative to the oxygen content of atmospheric conditions.

C228. The ophthalmic composition or method of any one of embodiments A1-A33, B1-B147, or C1-C227, wherein the ophthalmic composition disclosed herein has an oxygen content of less than 20 mol. %, less than 15 mol. %, less than 10 mol. %, less than 8 mol. %, less than 5 mol. %, less than 4 mol. %, less than 3 mol. %, less than 2.5 mol. %, or less than 1 mol. %, relative to the oxygen content of atmospheric conditions.

C229. The ophthalmic composition or method of any one of embodiments A1-A33, B1-B147, or C1-C228, wherein the ophthalmic composition disclosed herein has a reduced amount of dissolved oxygen content.

C230. The ophthalmic composition or method of any one of embodiments A1-A33, B1-B147, or C1-C229, wherein the ophthalmic composition disclosed herein has a dissolved oxygen content of less than 12 mg/L, less than 10 mg/L, less than 8 mg/L, less than 6 mg/L, less than 5 mg/L, less than 4 mg/L, less than 3 mg/L, less than 2 mg/L, or less than 1 mg/L.

C231. The ophthalmic composition or method of any one of embodiments A1-A33, B1-B147, or C1-C230, wherein the ophthalmic composition disclosed herein is stored in a container.

C232. The ophthalmic composition or method of any one of embodiments A1-A33, B1-B147, or C1-C231, wherein the ophthalmic composition disclosed herein is stored in a sealed container or sealable container.

C233. The ophthalmic composition or method of any one of embodiments A1-A33, B1-B147, or C1-C232, wherein the oxygen content of the head space within the container when storing the ophthalmic composition disclosed herein is a reduced oxygen content, relative to the oxygen content of atmospheric conditions.

C234. The ophthalmic composition or method of any one of embodiments A1-A33, B1-B147, or C1-C233, wherein the ophthalmic composition disclosed herein is purged with an inert gas prior to sealing within or enclosing within the container.

C235. The ophthalmic composition or method of any one of embodiments A1-A33, B1-B147, or C1-C234, wherein the head space within the container above the ophthalmic composition disclosed herein is purged with an inert gas to limit the oxygen content within the head space of the container storing the ophthalmic composition.

C236. The ophthalmic composition or method of any one of embodiments A1-A33, B1-B147, or C1-C235, wherein the dissolved oxygen content of the purged ophthalmic composition stored within the container upon sealing is less than 12 mg/L, less than 10 mg/L, less than 8 mg/L, less than 6 mg/L, less than 5 mg/L, less than 4 mg/L, less than 3 mg/L, less than 2 mg/L, or less than 1 mg/L.

C237. The ophthalmic composition or method of any one of embodiments A1-A33, B1-B147, or C1-C236, wherein the head space within the container above the ophthalmic composition is purged with an inert gas to limit the oxygen content of the head space of the container storing said ophthalmic composition.

C238. The ophthalmic composition or method of any one of embodiments A1-A33, B1-B147, or C1-C237, wherein the head space within the container above the ophthalmic composition disclosed herein is purged with an inert gas to limit the oxygen content within the head space of the container storing the ophthalmic composition to an oxygen content of less than 20 mol. %, less than 15 mol. %, such as an oxygen content of less than 10 mol. %, less than 8 mol. %, less than 5 mol. %, less than 4 mol. %, less than 3 mol. %, less than 2.5 mol. %, or less than 1 mol. %.

C239. The ophthalmic composition or method of any one of embodiments A1-A33, B1-B147, or C1-C238, wherein the head space within the container above the ophthalmic composition disclosed herein is purged with an inert gas to limit the oxygen content within the head space of the container storing the ophthalmic composition to an oxygen content of less than 12 mg oxygen gas/L inert gas, less than 10 mg oxygen gas/L inert gas, less than 8 mg oxygen gas/L inert gas, less than 6 mg oxygen gas/L inert gas, less than 5 mg oxygen gas/L inert gas, less than 4 mg oxygen gas/L inert gas, less than 3 mg oxygen gas/L inert gas, less than 2 mg oxygen gas/L inert gas, or less than 1 mg oxygen gas/L inert gas.

C240. The ophthalmic composition or method of any one of embodiments A1-A33, B1-B147, or C1-C239, wherein the head space within the container above the ophthalmic composition disclosed herein is purged with an inert gas to limit the oxygen content within the head space of the container storing the ophthalmic composition to an oxygen content of less than than 12 mL oxygen gas/L inert gas, less than 10 mL oxygen gas/L inert gas, less than 8 mL oxygen gas/L inert gas, less than 6 mL oxygen gas/L inert gas, less than 5 mL oxygen gas/L inert gas, less than 4 mL oxygen gas/L inert gas, less than 3 mL oxygen gas/L inert gas, less than 2 mL oxygen gas/L inert gas, or less than 1 mL oxygen gas/L inert gas.

C241. The ophthalmic composition or method of any one of embodiments A1-A33, B1-B147, or C1-C240, wherein the head space within the container above the ophthalmic composition and the ophthalmic composition stored therein are purged with an inert gas to limit the oxygen content of both the ophthalmic composition and the head space of the container storing said ophthalmic composition.

C242. The ophthalmic composition or method of any one of embodiments A1-A33, B1-B147, or C1-C241, wherein the head space within the container above the ophthalmic composition and the ophthalmic composition disclosed herein that is stored in the container are purged with an inert gas prior to sealing or enclosing the ophthalmic composition within said container such that the resulting dissolved oxygen content of the purged ophthalmic composition while stored within the sealed or enclosed container is less than 12 mg/L, less than 10 mg/L, less than 8 mg/L, less than 6 mg/L, less than 5 mg/L, less than 4 mg/L, less than 3 mg/L, less than 2 mg/L, or less than 1 mg/L.

C243. The ophthalmic composition or method of any one of embodiments A1-A33, B1-B147, or C1-C242, wherein the sealed container or sealable container is a bottle, a vial, or a syringe.

C244. The ophthalmic composition or method of any one of embodiments A1-A33, B1-B147, or C1-C243, wherein the sealed container or sealable container is a bottle.

C245. The ophthalmic composition or method of any one of embodiments A1-A33, B1-B147, or C1-C244, wherein the bottle is a glass or plastic bottle.

C246. The ophthalmic composition or method of any one of embodiments A1-A33, B1-B147, or C1-C245, wherein the bottle is sealed with a syringe needle piercable cap.

C247. The ophthalmic composition or method of any one of embodiments A1-A33, B1-B147, or C1-C246, wherein the sealed container or sealable container is a vial.

C248. The ophthalmic composition or method of any one of embodiments A1-A33, B1-B147, or C1-C247, wherein the vial is a glass or plastic vial.

C249. The ophthalmic composition or method of any one of embodiments A1-A33, B1-B147, or C1-C248, wherein the vial is sealed with a syringe needle piercable cap.

C250. The ophthalmic composition or method of any one of embodiments A1-A33, B1-B147, or C1-C249, wherein the sealed container or sealable container is a syringe.

C251. The ophthalmic composition or method of any one of embodiments A1-A33, B1-B147, or C1-C250, wherein the syringe containing the ophthalmic composition disclosed herein is vacuum sealed to limit or avoid oxygen exposure to said composition.

C252. The ophthalmic composition or method of any one of embodiments A1-A33, B1-B147, or C1-C251, wherein the bottle has a volume of 100 mL, 75 mL, 50 mL, 25 mL, 20 mL, 10 mL, 5 mL, or 1 mL.

C253. The ophthalmic composition or method of any one of embodiments A1-A33, B1-B147, or C1-C252, wherein the vial has a volume of 100 mL, 75 mL, 50 mL, 25 mL, 20 mL, 10 mL, 5 mL, or 1 mL.

C254. The ophthalmic composition or method of any one of embodiments A1-A33, B1-B147, or C1-C253, wherein the syringe is a 20 mL syringe, 10 mL syringe, 5 mL syringe, 4 mL syringe, 3 mL syringe, 2 mL syringe, 2.5 mL syringe, 1 mL syringe, or 0.5 mL syringe.

C255. The ophthalmic composition or method of any one of embodiments A1-A33, B1-B147, or C1-C254, wherein the inert gas is nitrogen gas or argon gas.

All publications, patents, and patent applications, mentioned in this specification are herein incorporated by reference in their entirety to the same extent as if each individual publication, patent, or patent application, was specifically and individually indicated to be incorporated by reference in its entirety.

It will be understood that the embodiments disclosed and defined in this specification extends to all alternative combinations of two or more of the individual features mentioned or evident from the text or drawings. All of these different combinations constitute various alternative aspects of the present disclosure.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method for ocular surgery in a patient in need thereof, comprising:
i) instilling an ophthalmic composition comprising Indigo Carmine into the patient's eye;
ii) performing the ocular surgery of the patient's instilled eye; and
iii) introducing a plurality of ophthalmic devices into the instilled eye.

2. The method of claim 1, wherein the ocular surgery includes or is glaucoma surgery.

3. The method of claim 2, wherein an ocular drainage structure is identified in the instilled patient's eye.

4. The method of claim 3, wherein the ocular drainage structure identified in the instilled patient's eye around the circumference of the drainage angle is selected from the group consisting of: a fine vessel, an aqueous vein, an episcleral vein, an interscleral channel (e.g., collector channel), a collector channel, a collector channel/aqueous/episcleral vein system, an aqueous drainage system, a conjunctival venous system, a subconjunctival space, a deep scleral plexus, a deep scleral plexus visually identifiable once a conjunctiva is reflected away, a trabecular meshwork, a canal of Schlemm, a suprachoroidal space, a scleral spur, anterior capsule of a crystalline lens, cornea, lens capsule, a retinal membrane, a corneal endothelial membrane, and Descemet's membrane.

5. The method of claim 4, wherein the identified ocular drainage structure is a collector channel.

6. The method of claim 4, wherein the identified ocular drainage structure is the canal of Schlemm.

7. The method of claim 4, wherein the identified ocular drainage structure is the trabecular meshwork.

8. The method of claim 4, wherein the identified ocular drainage structure is the suprachoroidal space.

9. The method of claim 3, wherein at least 2 or at least 3 ocular drainage structures are identified in the instilled patient's eye.

10. The method of claim 3, wherein the plurality of ophthalmic devices are positioned in a plurality of directions around the circumference of the drainage angle.

11. The method of claim 3, wherein the plurality of ophthalmic devices positioned around the circumference of the drainage angle are placed in, proximate, or adjacent to at least 2 or at least 3 of the ocular drainage structures identified in the instilled patient's eye.

12. The method of claim 3, wherein the plurality of ophthalmic devices are distributed among 3-6 ocular drainage structures identified in the instilled patient's eye around the circumference of the drainage angle, and positioned in, near or adjacent to said identified ocular drainage structures.

13. The method of claim 3, wherein the plurality of ophthalmic devices are positioned around the circumference of the drainage angle to maintain physiological aqueous humor circulation, maintain corneal nutrition, maintain corneal endothelial cell density and/or mitigate corneal endothelial cell loss.

14. The method of claim 3, wherein the plurality of ophthalmic devices introduced into the instilled eye are positioned around the circumference of the drainage angle to maintain physiological aqueous humor circulation of the patient's eye following the ocular surgery.

15. The method of claim 3, wherein the positioning of the plurality of ophthalmic devices in, near or adjacent to identified ocular drainage structures located around the circumference of the drainage angle in the instilled patient's eye maintains an average IOP of 10 mmHg of the treated patient's eye.

16. The method of claim 3, wherein the placement of the ophthalmic device near, proximate, or adjacent to, the identified ocular structure is within a distance of 1-5 inner diameters of said ophthalmic device.

17. The method of claim 3, wherein the ophthalmic device is a stent.

18. The method of claim 17, wherein at least 1 or at least 2 stents of the plurality of stents are placed in, proximate, or adjacent to, 1 or 2 identified ocular drainage structures located approximately opposite a main collector channel.

19. The method of claim 3, wherein the patient's density of corneal endothelium cells following introduction of the plurality of ophthalmic devices maintains and/or achieves an average of between 3000-4000 cells per square mm.

20. A method for maintaining physiological aqueous humor circulation of an eye of a patient following ocular surgery of the patient's eye, comprising:
i) instilling an ophthalmic composition comprising Indigo Carmine into the patient's eye;
ii) performing the ocular surgery of the patient's instilled eye;
iii) optionally, further instilling the ophthalmic composition comprising Indigo Carmine into the patient's eye following the ocular surgery; and
iv) introducing a plurality of ophthalmic devices into the instilled eye, wherein the plurality of ophthalmic devices are positioned around the circumference of the drainage angle.

21. The method of claim 19, wherein the ocular surgery includes or is glaucoma surgery.

22. The method of claim 21, wherein the ophthalmic device is a stent.
